SEQUENCE LISTING (92 sequences)

SEQ ID NO: 1 - protein, 330 aa, Homo Sapiens [sequence omitted]

SEQ ID NO: 2 - protein, 330 aa, Artificial Sequence [sequence omitted]

SEQ ID NO: 3 - protein, 242 aa, Artificial Sequence
  V-IGG1-Fc
GGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 330 aa, Artificial Sequence
  V-IGG1-A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 5 - protein, 242 aa, Artificial Sequence
  V-IGG1-A-Fc
GGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 6 - protein, 330 aa, Artificial Sequence
  V-IGG1-B
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 7 - protein, 242 aa, Artificial Sequence
  V-IGG1-B-Fc
GGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 8 - protein, 343 aa, Homo Sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 327 aa, Artificial Sequence
  V-IGG3
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVESKYGPPCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 10 - protein, 242 aa, Artificial Sequence
  V-IGG3-Fc
GGGGSGGGGSGGGGSKYGPPCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 11 - protein, 327 aa, Artificial Sequence
  V-IGG3-A
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVESKYGPPCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSRLTVDKSRWQQGNIFSCSVMHEALHNRFTQKSLSLSPGK

SEQ ID NO: 12 - protein, 242 aa, Artificial Sequence
  V-IGG3-A-Fc
GGGGSGGGGSGGGGSKYGPPCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSRLTVDKSRWQQGNIFSCSVMHEALHNRFTQKSLSLSPGK

SEQ ID NO: 13 - protein, 327 aa, Artificial Sequence
  V-IGG3-B
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVESKYGPPCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFLLYSKLTVDKSRWQQGNIFSCSVMHEALHNRFTQKSLSLSPGK

SEQ ID NO: 14 - protein, 242 aa, Artificial Sequence
  V-IGG3-B-Fc
GGGGSGGGGSGGGGSKYGPPCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFLLYSKLTVDKSRWQQGNIFSCSVMHEALHNRFTQKSLSLSPGK

SEQ ID NO: 15 - protein, 330 aa, Artificial Sequence
  V-IGG3-C
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTGDTTHTCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 16 - protein, 245 aa, Artificial Sequence
  V-IGG3-C-Fc
GGGGSGGGGSGGGGSKTGDTTHTCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 17 - protein, 330 aa, Artificial Sequence
  V-IGG3-D
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTGDTTHTCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSRLTVDKSRWQQGNIFSCSVMHEALHNRFTQKSLSLSPGK

SEQ ID NO: 18 - protein, 245 aa, Artificial Sequence
  V-IGG3-D-Fc
GGGGSGGGGSGGGGSKTGDTTHTCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSRLTVDKSRWQQGNIFSCSVMHEALHNRFTQKSLSLSPGK

SEQ ID NO: 19 - protein, 330 aa, Artificial Sequence
  V-IGG3-E
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTGDTTHTCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFLLYSKLTVDKSRWQQGNIFSCSVMHEALHNRFTQKSLSLSPGK

SEQ ID NO: 20 - protein, 245 aa, Artificial Sequence
  V-IGG3-E-Fc
GGGGSGGGGSGGGGSKTGDTTHTCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFLLYSKLTVDKSRWQQGNIFSCSVMHEALHNRFTQKSLSLSPGK

SEQ ID NO: 21 - protein, 255 aa, Artificial Sequence
  VCD3-H1L1
QVQLVQSGGGVVQPGRSLRLSCAASGYTFTRYTMHWVRQAPGKGLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGQGTKLEIK

SEQ ID NO: 22 - protein, 255 aa, Artificial Sequence
  VCD3-H1L1-DS
QVQLVQSGGGVVQPGRSLRLSCAASGYTFTRYTMHWVRQAPGKCLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGCGTKLEIK

SEQ ID NO: 23 - protein, 255 aa, Artificial Sequence
  VCD3-H1L2
QVQLVQSGGGVVQPGRSLRLSCAASGYTFTRYTMHWVRQAPGKGLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGQGTKLEIK

SEQ ID NO: 24 - protein, 255 aa, Artificial Sequence
  VCD3-H1L2-DS
QVQLVQSGGGVVQPGRSLRLSCAASGYTFTRYTMHWVRQAPGKCLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGCGTKLEIK

SEQ ID NO: 25 - protein, 255 aa, Artificial Sequence
  VCD3-H1L3
QVQLVQSGGGVVQPGRSLRLSCAASGYTFTRYTMHWVRQAPGKGLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIK

SEQ ID NO: 26 - protein, 255 aa, Artificial Sequence
  VCD3-H1L3-DS
QVQLVQSGGGVVQPGRSLRLSCAASGYTFTRYTMHWVRQAPGKCLEWVGYINPSRGYTNYNDSVKGRFTISTDKSKNTAYLQMNSLRAEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGCGTKLEIK

SEQ ID NO: 27 - protein, 255 aa, Artificial Sequence
  VCD3-H2L1
QVQLVQSGAEVKKPGASVKVSCKASGYTFTRYTMHWVRQAPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGQGTKLEIK

SEQ ID NO: 28 - protein, 255 aa, Artificial Sequence
  VCD3-H2L1-DS
QVQLVQSGAEVKKPGASVKVSCKASGYTFTRYTMHWVRQAPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGCGTKLEIK

SEQ ID NO: 29 - protein, 255 aa, Artificial Sequence
  VCD3-H2L2
QVQLVQSGAEVKKPGASVKVSCKASGYTFTRYTMHWVRQAPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGQGTKLEIK

SEQ ID NO: 30 - protein, 255 aa, Artificial Sequence
  VCD3-H2L2-DS
QVQLVQSGAEVKKPGASVKVSCKASGYTFTRYTMHWVRQAPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGCGTKLEIK

SEQ ID NO: 31 - protein, 255 aa, Artificial Sequence
  VCD3-H2L3
QVQLVQSGAEVKKPGASVKVSCKASGYTFTRYTMHWVRQAPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIK

SEQ ID NO: 32 - protein, 255 aa, Artificial Sequence
  VCD3-H2L3-DS
QVQLVQSGAEVKKPGASVKVSCKASGYTFTRYTMHWVRQAPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGCGTKLEIK

SEQ ID NO: 33 - protein, 255 aa, Artificial Sequence
  VCD3-H3L1
QVQLVQSGAEVKKPGASVKVSCKASGYTFTRYTMHWVRQSPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGQGTKLEIK

SEQ ID NO: 34 - protein, 255 aa, Artificial Sequence
  VCD3-H3L1-DS
QVQLVQSGAEVKKPGASVKVSCKASGYTFTRYTMHWVRQSPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSDIQMTQSPSSLSASVGDRVTITCQASSSVSYMNWYQQKPGKAPKRWIYDTSKLASGVPSRFSGSGSGTDYTFTISSLQPEDIATYYCQQWSSNPFTFGCGTKLEIK

SEQ ID NO: 35 - protein, 255 aa, Artificial Sequence
  VCD3-H3L2
QVQLVQSGAEVKKPGASVKVSCKASGYTFTRYTMHWVRQSPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGQGTKLEIK

SEQ ID NO: 36 - protein, 255 aa, Artificial Sequence
  VCD3-H3L2-DS
QVQLVQSGAEVKKPGASVKVSCKASGYTFTRYTMHWVRQSPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIVLTQSPATLSLSPGERATLSCRASSSVSYMNWYQQKPGQAPRRLIYDTSKRATGIPARFSGSGSGTDYTLTISSLEPEDAAVYYCQQWSSNPFTFGCGTKLEIK

SEQ ID NO: 37 - protein, 255 aa, Artificial Sequence
  VCD3-H3L3
QVQLVQSGAEVKKPGASVKVSCKASGYTFTRYTMHWVRQSPGQGLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGQGTKLEIK

SEQ ID NO: 38 - protein, 255 aa, Artificial Sequence
  VCD3-H3L3-DS
QVQLVQSGAEVKKPGASVKVSCKASGYTFTRYTMHWVRQSPGQCLEWMGYINPSRGYTNYNQKFQGRVTMTTDKSTSTAYMELSSLRSEDTAVYYCARYYDDHYSLDYWGQGTTVTVSSGGGGSGGGGSGGGGSGGGGSGGGGSGGGGSEIQLTQSPATLSLSPGERATLSCRASSSVSYMNWYQQKPGQAPRRWIYDTSKLATGIPARFSGSGSGTDYTLTISSLEPEDAAVYYCQQWSSNPFTFGCGTKLEIK

SEQ ID NO: 39 - protein, 330 aa, Homo Sapiens [sequence omitted]

SEQ ID NO: 40 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 327 aa, Artificial Sequence
  V-IGG4-A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 242 aa, Artificial Sequence
  V-IGG4-A-Fc
GGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 327 aa, Artificial Sequence
  V-IGG4-B
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 44 - protein, 242 aa, Artificial Sequence
  V-IGG4-B-Fc
GGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 45 - protein, 326 aa, Homo Sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 46 - protein, 327 aa, Artificial Sequence
  V-IGG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 47 - protein, 241 aa, Artificial Sequence
  V-IGG2-Fc
GGGGSGGGGSGGGGSDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 48 - protein, 327 aa, Artificial Sequence
  V-IGG2-A
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK

SEQ ID NO: 49 - protein, 241 aa, Artificial Sequence
  V-IGG2-A-Fc
GGGGSGGGGSGGGGSDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK

SEQ ID NO: 50 - protein, 327 aa, Artificial Sequence
  V-IGG2-B
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFLLYSKLTVDKSRWQQGNVFSCSVMHEALHNHYTQKSLSLSPGK

SEQ ID NO: 51 - protein, 241 aa, Artificial Sequence
  V-IGG2-B-Fc
GGGGSGGGGSGGGGSDKTHTCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFLLYSKLTVDKSRWQQGNVFSCSVMHEALHNHYTQKSLSLSPGK

SEQ ID NO: 52 - protein, 326 aa, Artificial Sequence
  V-IGG2-C
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 53 - protein, 240 aa, Artificial Sequence
  V-IGG2-C-Fc
GGGGSGGGGSGGGGSYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 54 - protein, 326 aa, Artificial Sequence
  V-IGG2-D
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK

SEQ ID NO: 55 - protein, 240 aa, Artificial Sequence
  V-IGG2-D-Fc
GGGGSGGGGSGGGGSYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFFLYSRLTVDKSRWQQGNVFSCSVMHEALHNHYTQKSLSLSPGK

SEQ ID NO: 56 - protein, 326 aa, Artificial Sequence
  V-IGG2-E
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFLLYSKLTVDKSRWQQGNVFSCSVMHEALHNHYTQKSLSLSPGK

SEQ ID NO: 57 - protein, 240 aa, Artificial Sequence
  V-IGG2-E-Fc
GGGGSGGGGSGGGGSYGPPCPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDISVEWESNGQPENNYKTTPPMLDSDGSFLLYSKLTVDKSRWQQGNVFSCSVMHEALHNHYTQKSLSLSPGK

SEQ ID NO: 58 - protein, 107 aa, Mus musculus
DIVMTQSHKFMSTSVGDRVSITCKASQDVSTAVAWYQQKPGQSPKLLIYWASTRHTGVPDRFTGSGSGTDYTLTISSVQAEDLALYYCQQHYSTPWTFGGGTKLEIK

SEQ ID NO: 59 - protein, 107 aa, Artificial Sequence
  NKTT120 VL
DIQMTQSPSSLSASVGDRVTITCKASQDVSTAVAWYQQKPGQAPRLLIYWASTRHTGVPSRFSGSGSGTDFTLTISSLQPEDFALYYCQQHYSTPWTFGQGTKLEIK

SEQ ID NO: 60 - protein, 107 aa, Artificial Sequence
  VTCR-L1
DIQMTQSPSSLSASVGDRVTITCRASQDVSTAVAWYQQKPGKAPKLLIYWASTRHTGVPSRFSGSGSGTDYTLTISSLQPEDFATYYCQQHYSTPWTFGGGTKVEIK

SEQ ID NO: 61 - protein, 122 aa, Mus musculus
EVKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTRNGNYVDYAMDYWGQGTSVTVSS

SEQ ID NO: 62 - protein, 122 aa, Artificial Sequence
  NKTT120 VH
EVQLVESGGGLVQPGGSLRLSCVASGFTFSNYWMNWVRQAPGKGLEWVAEIRLKSNNYATHYAESVKGRFTISRDDSKNTVYLQMNSLRAEDTAVYYCTRNGNYVDYAMDYWGQGTLVTVSS

SEQ ID NO: 63 - protein, 122 aa, Artificial Sequence
  VTCR-H1
EVQLVESGGGLVQPGGSLKLSCAASGFTFSNYWMNWVRQAPGKGLEWVAEIRLKSNNYATHYAESVKGRFTISRDDSKNTAYLQMNSLKTEDTAVYYCTRNGNYVDYAMDYWGQGTLVTVSS

SEQ ID NO: 64 - protein, 259 aa, Artificial Sequence
  VTCR-Chimeric-scFv
EVKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTRNGNYVDYAMDYWGQGTSVTVSSGGGGSGGGGSGGGGSGGGGSGGGGSGGGGSDIVMTQSHKFMSTSVGDRVSITCKASQDVSTAVAWYQQKPGQSPKLLIYWASTRHTGVPDRFTGSGSGTDYTLTISSVQAEDLALYYCQQHYSTPWTFGGGTKLEIK

SEQ ID NO: 65 - protein, 259 aa, Artificial Sequence
  VTCR-Chimeric-scFv-DS
EVKLEESGGGLVQPGGSMKLSCVASGFTFSNYWMNWVRQSPEKCLEWVAEIRLKSNNYATHYAESVKGRFTISRDDSKSSVYLQMNNLRAEDTGIYYCTRNGNYVDYAMDYWGQGTSVTVSSGGGGSGGGGSGGGGSGGGGSGGGGSGGGGSDIVMTQSHKFMSTSVGDRVSITCKASQDVSTAVAWYQQKPGQSPKLLIYWASTRHTGVPDRFTGSGSGTDYTLTISSVQAEDLALYYCQQHYSTPWTFGCGTKLEIK

SEQ ID NO: 66 - protein, 259 aa, Artificial Sequence
  VTCR-NKTT120-scFv
EVQLVESGGGLVQPGGSLRLSCVASGFTFSNYWMNWVRQAPGKGLEWVAEIRLKSNNYATHYAESVKGRFTISRDDSKNTVYLQMNSLRAEDTAVYYCTRNGNYVDYAMDYWGQGTLVTVSSGGGGSGGGGSGGGGSGGGGSGGGGSGGGGSDIQMTQSPSSLSASVGDRVTITCKASQDVSTAVAWYQQKPGQAPRLLIYWASTRHTGVPSRFSGSGSGTDFTLTISSLQPEDFALYYCQQHYSTPWTFGQGTKLEIK

SEQ ID NO: 67 - protein, 259 aa, Artificial Sequence
  VTCR-NKTT120-scFv-DS
EVQLVESGGGLVQPGGSLRLSCVASGFTFSNYWMNWVRQAPGKCLEWVAEIRLKSNNYATHYAESVKGRFTISRDDSKNTVYLQMNSLRAEDTAVYYCTRNGNYVDYAMDYWGQGTLVTVSSGGGGSGGGGSGGGGSGGGGSGGGGSGGGGSDIQMTQSPSSLSASVGDRVTITCKASQDVSTAVAWYQQKPGQAPRLLIYWASTRHTGVPSRFSGSGSGTDFTLTISSLQPEDFALYYCQQHYSTPWTFGCGTKLEIK

SEQ ID NO: 68 - protein, 259 aa, Artificial Sequence
  VTCR-H1L1-scFv
EVQLVESGGGLVQPGGSLKLSCAASGFTFSNYWMNWVRQAPGKGLEWVAEIRLKSNNYATHYAESVKGRFTISRDDSKNTAYLQMNSLKTEDTAVYYCTRNGNYVDYAMDYWGQGTLVTVSSGGGGSGGGGSGGGGSGGGGSGGGGSGGGGSDIQMTQSPSSLSASVGDRVTITCRASQDVSTAVAWYQQKPGKAPKLLIYWASTRHTGVPSRFSGSGSGTDYTLTISSLQPEDFATYYCQQHYSTPWTFGGGTKVEIK

SEQ ID NO: 69 - protein, 259 aa, Artificial Sequence
  VTCR-H1L1-scFv-DS
EVQLVESGGGLVQPGGSLKLSCAASGFTFSNYWMNWVRQAPGKCLEWVAEIRLKSNNYATHYAESVKGRFTISRDDSKNTAYLQMNSLKTEDTAVYYCTRNGNYVDYAMDYWGQGTLVTVSSGGGGSGGGGSGGGGSGGGGSGGGGSGGGGSDIQMTQSPSSLSASVGDRVTITCRASQDVSTAVAWYQQKPGKAPKLLIYWASTRHTGVPSRFSGSGSGTDYTLTISSLQPEDFATYYCQQHYSTPWTFGCGTKVEIK

SEQ ID NO: 70 - protein, 327 aa, Artificial Sequence
  V-IGG4-ADC-A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKCKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 242 aa, Artificial Sequence
  V-IGG4-ADC-A-Fc
GGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKCKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 327 aa, Artificial Sequence
  V-IGG4-ADC-B
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKCKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 73 - protein, 242 aa, Artificial Sequence
  V-IGG4-ADC-B-Fc
GGGGSGGGGSGGGGSKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCAVSNKGLPSSIEKTISKCKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 74 - protein, 5 aa, Artificial Sequence
  Linker sequence
GGGGS

SEQ ID NO: 75 - protein, 5 aa, Artificial Sequence
  CD3 VL CDR1
SSVSY

SEQ ID NO: 76 - protein, 3 aa, Artificial Sequence
  CD3 VL CDR2
DTS

SEQ ID NO: 77 - protein, 9 aa, Artificial Sequence
  CD3 VL CDR3
QQWSSNPFT

SEQ ID NO: 78 - protein, 8 aa, Artificial Sequence
  CD3 VH CDR1
GYTFTRYT

SEQ ID NO: 79 - protein, 8 aa, Artificial Sequence
  CD3 VH CDR2
INPSRGYT

SEQ ID NO: 80 - protein, 12 aa, Artificial Sequence
  CD3 VH CDR3
ARYYDDHYCLDY

SEQ ID NO: 81 - protein, 92 aa, Artificial Sequence
  TVA24_HUMAN T cell receptor alpha variable (residues 23-114)
ILNVEQSPQSLHVQEGDSTNFTCSFPSSNFYALHWYRWETAKSPEALFVMTLNGDEKKKGRISATLNTKEGYSYLYIKGSQPEDSATYLCAF

SEQ ID NO: 82 - protein, 21 aa, Artificial Sequence
  Sequence of TRAJ18, T cell receptor alpha joining 18
DRGSTLGRLYFGRGTQLTVWP

SEQ ID NO: 83 - protein, 113 aa, Artificial Sequence
  VA24JA18
ILNVEQSPQSLHVQEGDSTNFTCSFPSSNFYALHWYRWETAKSPEALFVMTLNGDEKKKGRISATLNTKEGYSYLYIKGSQPEDSATYLCAFDRGSTLGRLYFGRGTQLTVWP

SEQ ID NO: 84 - protein, 6 aa, Artificial Sequence
  6B11 VL CDR1
QDVSTA

SEQ ID NO: 85 - protein, 3 aa, Artificial Sequence
  6B11 VL DR2
WAS

SEQ ID NO: 86 - protein, 9 aa, Artificial Sequence
  6B11 VL CDR3
QQHYSTPWT

SEQ ID NO: 87 - protein, 8 aa, Artificial Sequence
  6B11 VH CDR1
GFTFSNYW

SEQ ID NO: 88 - protein, 10 aa, Artificial Sequence
  6B11 VH CDR2
IRLKSNNYAT

SEQ ID NO: 89 - protein, 13 aa, Artificial Sequence
  6B11 VH CDR3
TRNGNYVDYAMDY

SEQ ID NO: 90 - protein, 453 aa, Homo Sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 91 - protein, 349 aa, Artificial Sequence
  V-IGM
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 92 - protein, 5 aa, Artificial
  Gly-Ser linker
GGGGS

(19) United States
(12) Patent Application Publication
(10) Pub. No.: US 2023/0338495 A1
(43) Pub. Date: Oct. 26, 2023

(54) Vaccine Compositions and Antibodies For Lyme Disease

(71) Applicant: Vitruviae LLC, Nutley, NJ (US)

(72) Inventors: Mahiuddin AHMED, Verona, NJ (US); Sonia SEQUEIRA, Verona, NJ (US); Julien DUGAL-TESSIER, Belmont, MA (US); Bram Pieter KARSTEN, Teaneck, NJ (US); Nareshkumar JAIN, Flemington, NJ (US)

(21) Appl. No.: 18/022,087

(22) PCT Filed: Aug. 19, 2021
(86) PCT No.: PCT/US2021/046693

§ 371 Date: Feb. 17, 2023

Related U.S. Application Data
(60) Provisional application No. 63/067,598, filed on Aug. 19, 2020.

(51) Int. Cl. A61K 39/00; C07H 3/02; C07K 16/44; C07K 16/28; A61P 31/04; C07K 16/00; G01N 33/569

(52) U.S. Cl. CPC A61K 39/0013; A61P 31/04; C07H 3/02; C07K 16/005; C07K 16/2803; C07K 16/2809; C07K 16/283; C07K 16/44; G01N 33/56911; A61K 2039/505; A61K 2039/55555; A61K 2039/55583; A61K 2039/6081; A61K 2039/70; C07K 2317/14; C07K 2317/20; C07K 2317/52; C07K 2317/522; C07K 2317/524; C07K 2317/526; C07K 2317/53; C07K 2317/55; C07K 2317/565; C07K 2317/622; C07K 2317/73; C07K 2317/732; C07K 2317/734; G01N 2333/20; G01N 2405/00; G01N 2800/52

(57) ABSTRACT

The present invention relates to vaccine compositions comprising lipid antigens, antibodies targeting lipid antigens, pharmaceutical compositions comprising such and their use in diagnosing, monitoring, treating, and preventing infectious disease, such as Lyme disease. In one aspect, administered is a therapeutically effective amount of a vaccine composition comprising a lipid antigen, an antibody or fragment thereof binding a lipid antigen, and/or a pharmaceutical composition comprising an antibody or fragment thereof binding a lipid antigen. Other aspects are described.

The present invention relates to methods of prophylaxis, diagnosis and treatment of Lyme disease. In particular, this invention relates to composition of vaccines consisting of synthetic Borrelia glycolipid antigens BBGL-1 and BBGL-2, its formulation and adjuvants, including B-glucan. This invention further relates to the route of administration, dose, and schedule of such vaccines. This invention relates to anti-BBGL-1 and anti-BBGL-2 antibodies for the treatment of Lyme disease and a specific composition, formulation, route of administration, dose, and schedule. Further, it relates to the use of these antibodies in preclinical models of the disease and for selection of subjects that may benefit from treatment with such antibodies. The use of anti-BBGL-1 and BBGL-2 antibodies for imaging of infection progression, and alternatively, anti-BBGL-1/CD3, anti-BBGL-2/CD3, anti-BBGL-1/CD56, anti-BBGL-2/CD56 bispecific antibodies. Furthermore, to a CD3-CD56 construct for NKT cell interaction. Finally, this invention relates to a blood biomarker (BBGL-½) for rapid and easy monitoring of response of treatment, and a related screening test and diagnostic kit.

### TECHNICAL BACKGROUND

### Lyme Disease

Lyme disease is a bacterial infection spread to humans by the bite of ticks, mosquitos, spiders, and fleas, that causes at least 30,000 illnesses each year in the United States and over 100,000 in Europe. (CDC website, www_euro.who.int/data/assets/pdf_file/0006/96819/E89522.pdf). It has been estimated that due to lack of or inaccurate testing, there may be over 1.5 million cases per year, globally (globallymealliance.org/about-lyme/).

Lyme disease is the most commonly occurring and fastest growing vector-borne infectious bacterial disease in the United States.

Lyme disease is caused by the gram-negative spirochete bacteria group Borrelia Burgdorferi sensu lato which includes hundreds of strains. These strains are transmitted by different species of ticks and are endemic to North America (such as B. burgdorferi and mayonii), Europe (such as B. burgdorferi, B. afzelii and B. garinii) and Asia (such as B. afzelii and B. garinii).

Lyme disease is a multi-systemic disorder that affects the skin, nervous system, heart and joints. In the acute phase, B. burgdorferi (serotype 1) and B. mayonii infection are associated with fever, headache, rash, and neck pain in the days after infection and can cause arthritis after a few weeks of illness. Unlike B. burgdorferi, B. mayonii can also cause nausea, vomiting, large, widespread rashes, and results in a higher concentration of bacteria in the blood. B. afzelii (serotype 2) is mostly associated with skin infections and B. garinii (serotype 3,4, and 6) is neurogenic (Steere 2016).

OspA is a 28.5 kDa surface exposed lipoprotein which is attached to the outer membrane by its N-terminal lipid moiety. The C-terminal half is more distant from the bacterial surface and more accessible for anti-OspA antibodies. OspA covers other conserved surface proteins such as P13 and P66 protecting them from antibody recognition. OspA induces strong anti-OspA IgG antibody titers, when adjuvated (Nigrovic and Thompson 2007). However, significant cross-reactivity to human proteins has been related to autoimmune responses (Trollmo 2001, Steere 2011). Furthermore, OspA protein expression and amino acid sequence is highly variable among known Borrelia strains, requiring a different antigen for each currently known strain, as well as emerging strains. Notably, OspA levels are undetectable in humans after infection. This is due to the fact that OspA binds to TROSPA (tick receptor for OspA) in the bacteria infected tick gut and is downregulated during tick feeding, allowing the spirochetes to penetrate the gut epithelium, migrate to the salivary glands and further into the blood of the host. Therefore, the mechanism of OspA vaccines as well as the structurally similar OspB, involves transfer of antibodies to ticks, preventing transmission of the organism to the host.

Outer surface protein C (OspC) is a 22 kDa surface-exposed lipoprotein (Fuchs et al., 1992) that is highly immunogenic and an essential virulence factor at the tick-host interface but downregulated in the host after infection (Hovius et al., 2008; Earnhart et al., 2010; Onder et al., 2012). Over 30 distinct OspC phyletic types have been identified (Seinost et al., 1999; Wang et al., 1999; Brisson and Dykhuizen, 2004; Earnhart and Marconi, 2007).

### BBGL-1 and BBGL-2

Borrelia bacteria strains are characterized by a bilayer membrane composed of 2 types of glycolipids (cholesteryl 6-O-palmitoyl-β-D-galactopyranoside, also known as BBGL-1 and 1-oleoyl-2-palmitoyl-3-(a-D-galactosyl)-sn-glycerol or BBGL-2; see FIG. 1), 2 types of phospholipid (phosphatidylcholine and phosphatidylglycerol) and several transmembrane and surface lipoproteins, including OspA (Hossein 2001 and Toledo 2011). No polysaccarides or lipopolysaccarides have been detected to date.

### Immune Cell Response

Jones et al 2006 observed low numbers of NKT cells in infected joint tissue of Lyme disease patients and almost none were found within heart tissue. In mice, deletion of NKT cells resulted in more severe and prolonged arthritis and reduced ability to clear spirochetes from tissues (Kumar 2000).

Kinjo et al (2006) showed in a Lyme disease animal model that invariant natural killer T cells recognize BBGL-2 presented by CD1d on dendritic cells, resulting in NKT proliferation and cytokine release.

In contrast to peptide-recognizing T cells, invariant natural killer T cells (NKT cells) express a semi-invariant T cell receptor that specifically recognizes self- or foreign-lipids presented by CD1d molecules. There are three major functionally distinct innate effector states for NKT cells conferring early protective immunity against pathogens through cytokines release within hours, as well as activation of cytotoxic T cells without APC presentation. Growing evidence suggests that NKT cells play a role in tissue homeostasis (Van Kaer 2015, Teige 2010, Selvanantham 2016, Wang 2014). Natural killer cells are hybrid innate and adaptive immune cells (Qin 2019). They act within hours of an infection and specifically recognize glycolipids presented by CD1d, a non-classical antigen presenting molecule.

NKT cells express NK markers NK 1.1, CD16 and CD56 and T cell marker CD3.

High expression of CD56 is associated with a strong immunostimulatory effect in infections, autoimmune diseases, and cancer (Van Ackerman 2017, Lugli 2014). CD56 CAR T cells (NCT03473496) and antibody-drug conjugates against CD56 have been evaluated in oncology clinical trials (NCT02452554, Socinski 2016).

### SUMMARY OF THE INVENTION

Lyme disease can lead to severe disease burden and death related to carditis. Psychological symptoms such as dementia, cognitive impairment, depression, and suicidal thoughts have been frequently reported by both adults and children (Fallon 1994, Rebman 2018, Kalish 2001, Tagar 2001). Thus, there is a need for safe and efficient means for preventing, diagnosing and/or treating Lyme disease.

Lyme borreliosis has been coined the “great imitator” since the symptoms are vague rendering the clinical diagnosis challenging. Diagnosis of Lyme disease is made based on the combination of symptoms, two-tiered ELISA and Western blot for antibodies, however the sensitivity and accuracy of these tests is suboptimal. Thus, there is a need for improved diagnostics and means for diagnosing Lyme disease.

In a recent study, 97% of patients in a 6000-patient study reported persistent symptoms including fatigue, sleep impairment, nerve pain, headaches, memory loss, joint pain, and depression, of which 60% were severe. More than 40% of patients are unable to work due to their disease and symptoms can last months to years (Johnson 2018, also Berende 2016, Cairns 2005). In addition, people with HLA-DR2 or DR4 alleles involved in the positive and negative selection of immature T cells and that determine the type of cellular immune response that is initiated, have a genetic predisposition for the development of antibiotic-resistant Lyme borreliosis (Iliopoulou, 2009, Kalish 1993, Ball 2009). 25% of new cases of Lyme disease are children between the age of 5 and 14. Thus, there is a need for safe and efficient means for treating Lyme disease and any symptoms derived from Lyme disease, including any persistent symptoms.

A human vaccine for Lyme disease is warranted. The only FDA approved vaccine to date is Smith Kline’s LymErix, later withdrawn by the pharmaceutical company due to public concerns of disease-like side-effects such as arthritis as well as sub-optimal efficacy (pivotal trial 5,469 recipients ages 15 to 70 years, 3 doses at 0, 1 and 12 months followed for 20 months). Thus, there is a need for safe and efficient vaccine for preventing and/or treating Lyme disease.

Another vaccine, ImmuLyme by Aventis Pasteur, advanced to Phase III evaluation however was not pursued. Valneva/Pfizer are currently evaluating VLA15, a multiple-antigen vaccine in a phase 2 clinical trial (NCT03970733, Earnhardt and Marconi 2007) and Lyme PreEP is a prophylactic antibody in development by Mass Biologics. All these approaches target the Borrelia membrane outer surface protein A (OspA). Although OspA, as well as other lipoproteins are immunogenic, their remarkable genetic variability, changing protein expression throughout the enzootic cycle and possibly pleomorphic forms (spirochetes, round bodies, cysts) challenge vaccine development.

Thus, there is a need for safe, reliable, and efficient vaccine for preventing and/or treating Lyme disease.

VLA15 is an aluminium hydroxide adjuvanted multivalent outer surface protein A(OspA) -based vaccine intended to address earlier issues with OspA vaccines that is currently in clinical investigation (NCT01504347). The vaccine includes three proteins with 2 antigens each, each containing the C-terminal half of two OspA serotypes linked to form a heterodimer.

The use of cocktails consisting of multiple recombinant OspC proteins has not proven effective however chimeritopes (chimeric epitope-based proteins), that consist of diverse L5 and H5 epitopes elicit bactericidal and protective Ab responses (Earnhart and Marconi, 2008; Marconi and Earnhart, 2010). VANGUARD®crLyme (Zoetis), the most recent vaccine to receive USDA approval for prevention of Lyme disease in dogs consists of a OspA and a multivalent OspC chimeritope (Rhodes 2014). Thus, there is a need for safe and efficient means for preventing, diagnosing and/or treating Lyme disease.

In general, only a few antibodies have been approved by the FDA to treat antibiotic resistant ESKAPEE bacterial infections (Zurawski 2020). Thus, there is a need for safe and efficient antibody therapeutics for preventing, diagnosing and/or treating bacterial infections.

There are no approved therapies for Lyme disease. Standard treatment consists of antibiotics (doxycycline, amoxicillin, cefuroxime axetil and intravenous ceftriaxone or penicillin). Although patients treated in the early stages of Lyme disease may recover symptomatically, there are no reliable tests to demonstrate eradication of disease and some patients have persistent or worsening synovitis or antibiotic refractory arthritis which may be caused directly by the disease or subsequent autoimmune activity. Therefore, there is an urgent unmet need to specifically and accurately screen, treat and monitor Lyme disease.

The present inventors surprisingly discovered that vaccines targeting a lipid antigen might be developed. They further discovered that oral or nasal delivery of vaccines targeting a lipid antigen is immunogenic and might increase compliance and provide a safer method of vaccine delivery.

According to an aspect, the invention concerns a method of preventing, treating and/or alleviating an infectious disease comprising administering to a human or animal patient in need thereof a therapeutically effective amount of
a. a vaccine composition comprising a lipid antigen,
b. an antibody or fragment thereof binding a lipid antigen, and/or
c. a pharmaceutical composition comprising an antibody or fragment thereof binding a lipid antigen.

According to another aspect, the invention concerns a method of preventing, treating and/or alleviating Lyme disease comprising administering to a patient in need thereof a therapeutically effective amount of
a. a vaccine composition comprising a lipid antigen,
b. an antibody or fragment thereof binding a lipid antigen, and/or
c. a pharmaceutical composition comprising an antibody or fragment thereof binding a lipid antigen.

According to another aspect, the invention concerns a method for producing an antibody or fragment thereof binding a lipid antigen
(i) Providing a vaccine composition comprising a lipid antigen,
(ii) Administering said vaccine composition to an animal or human, to trigger or promote the production of an antibody or fragment thereof binding to said lipid antigen,
(iii) Obtaining said antibody or fragment thereof binding a lipid antigen from said animal or human.

According to another aspect, the invention concerns a vaccine composition comprising a lipid antigen.

According to another aspect, the invention concerns a method for prevention, treatment and/or alleviation of Lyme disease by administering the vaccine according to the invention to a subject in need thereof.

According to another aspect, the invention concerns an antibody or fragment thereof binding a lipid antigen.

According to another aspect, the invention concerns a method of producing an antibody or fragment thereof according to the invention, wherein said antibody or fragment thereof is produced by animal immunization.

According to another aspect, the invention concerns a method of producing an antibody or fragment thereof according to the invention comprising
a. Isolating B cells from a Lyme diseases human or animal patient,
b. Isolating B-cell receptors from said B cells,
c. Sequencing said B-cell receptors, and
d. Cloning said B-cell receptors.

According to another aspect, the invention concerns a method of producing an antibody or fragment thereof according to the invention using a human antibody phage display library.

According to another aspect, the invention concerns an antibody or fragment thereof obtainable by the method of the invention.

According to another aspect, the invention concerns a pharmaceutical composition comprising an antibody or fragment thereof according to the invention, optionally comprising one or more excipients such as diluents, binders or carriers.

According to another aspect, the invention concerns an isolated nucleic acid molecule encoding an antibody or fragment thereof according to the invention.

According to another aspect, the invention concerns a recombinant vector comprising the nucleic acid molecule of the invention.

According to another aspect, the invention concerns a host cell comprising the recombinant vector of the invention.

According to another aspect, the invention concerns a method to produce an antibody or fragment thereof according to the invention comprising a step of culturing the host cell according to the invention in a culture medium under conditions allowing the expression of the antibody or fragment thereof and separating the antibody or fragment thereof from the culture medium.

According to another aspect, the invention concerns a method to produce an antibody or fragment thereof according to the invention comprising a synthetic and/or recombinant step.

According to another aspect, the invention concerns use of an antibody or fragment thereof or vaccine composition according to the invention in a preclinical model.

According to another aspect, the invention concerns a method of preventing, treating and/or alleviating an infectious disease comprising administering to a patient in need thereof a therapeutically effective amount of the antibody or fragment thereof or pharmaceutical composition according to the invention.

According to another aspect, the invention concerns a method of preventing, treating and/or alleviating a condition related to and/or caused by Lyme disease comprising administration to a patient in need thereof a therapeutically effective amount of the antibody or fragment thereof or pharmaceutical composition according to the invention.

According to another aspect, the invention concerns a method of preventing, treating and/or alleviating Lyme disease comprising administering to a patient in need thereof a therapeutically effective amount of the antibody or fragment thereof or pharmaceutical composition according to the invention.

According to another aspect, the invention concerns a method of diagnosing Lyme disease comprising administering to a patient in need thereof a therapeutically effective amount of the antibody or fragment thereof or pharmaceutical composition according to the invention.

According to another aspect, the invention concerns a method of diagnosing Lyme disease comprising the steps of
a. obtaining a blood or urine sample from a subject, and
b. testing said blood or urine sample for the presence of a BBGL-1 and/or BBGL-2 biomarker with the antibody or fragment thereof according to the invention.

According to another aspect, the invention concerns a method of selecting and/or identifying a subject that might benefit from treatment with an antibody or fragment thereof or vaccine according to the invention comprising the steps of
a. obtaining a blood or urine sample from a subject, and
b. testing said blood or urine sample for the presence of a BBGL-1 and/or BBGL-2 biomarker with the antibody or fragment thereof according to the invention.

According to another aspect, the invention concerns a method of monitoring infectious disease progression and/or disease resolution comprising the steps of
a. obtaining a blood or urine sample from a subject, and
b. testing said blood or urine sample for the presence of a BBGL-1 and/or BBGL-2 biomarker with the antibody or fragment thereof according to the invention.

According to another aspect, the invention concerns a method of in vivo imaging of bacterial infections comprising an antibody or fragment thereof according to the invention.

According to another aspect, the invention concerns a diagnostic kit comprising an antibody or fragment thereof according to the invention and instructions for use.

According to another aspect, the invention concerns a kit for screening comprising an antibody or fragment thereof according to the invention and instructions for use.

According to another aspect, the invention concerns an antibody or fragment thereof, preferably according to the invention, capable of binding a TCR Va24Ja18 antigen, wherein said antibody is an NKT cell engaging antibody; and preferably wherein said antibody or fragment thereof is capable of binding an additional antigen, such as a humanized 6B11 antigen, preferably according to the description.

According to another aspect, the invention concerns an antibody or fragment thereof, preferably according to the invention, capable of binding a CD3 antigen, and wherein said antibody or fragment further is capable of binding a CD16 and/or a CD56 antigen; and preferably wherein said antibody or fragment thereof is capable of binding an additional antigen allowing NKT cells to be re-directed to said additional antigen.

According to another aspect, the invention concerns an antibody or fragment thereof, binding a TCR Va24Ja18 antigen, wherein said antibody is an NKT cell engaging antibody,
wherein said antibody or fragment thereof further binds a CD3 antigen, and
wherein said antibody or fragment thereof further binds a CD16 and/or a CD56 antigen.

### DETAILED DISCLOSURE

Certain embodiments of the invention relate to methods of treatment.

According to an embodiment, the invention concerns a method of preventing, treating and/or alleviating an infectious disease comprising administering to a patient in need thereof a therapeutically effective amount of
a. a vaccine composition comprising a lipid antigen,
b. an antibody or fragment thereof binding a lipid antigen, and/or
c. a pharmaceutical composition comprising an antibody or fragment thereof binding a lipid antigen.

According to an embodiment, the invention concerns a method of preventing, treating and/or alleviating Lyme disease comprising administering to a patient in need thereof a therapeutically effective amount of
a. a vaccine composition comprising a lipid antigen,
b. an antibody or fragment thereof binding a lipid antigen, and/or
c. a pharmaceutical composition comprising an antibody or fragment thereof binding a lipid antigen.

According to an embodiment, the invention concerns a method for producing an antibody or fragment thereof binding a lipid antigen comprising
(i) Providing a vaccine composition comprising a lipid antigen,
(ii) Administering said vaccine composition to an animal or human, to trigger or promote the production of an antibody or fragment thereof binding to said lipid antigen,
(iii) Obtaining said antibody or fragment thereof binding a lipid antigen from said animal or human.

Certain embodiments of the invention relate to vaccines.

According to an embodiment, the invention concerns a vaccine composition comprising a lipid antigen.

According to an embodiment, the invention concerns the vaccine composition, wherein said lipid antigen is a glycolipid antigen.

According to an embodiment, the invention concerns the vaccine composition, wherein said lipid antigen is a BBGL-1 and/or BBGL-2 antigen.

According to an embodiment, the invention concerns the vaccine composition, wherein said antigen is synthetically produced.

According to an embodiment, the invention concerns the vaccine composition, wherein said antigen is encapsulated in a vesicle comprising at least one lipid layer.

According to an embodiment, the invention concerns the vaccine composition, wherein said vesicle comprises at least one lipid bilayer.

According to an embodiment, the invention concerns the vaccine composition, wherein said vesicle is selected from the group consisting of solid lipid nanoparticles (SLNs), emulsions, liposomes, micelles, and bilayer sheets.

According to an embodiment, the invention concerns the vaccine composition, wherein said vesicle comprises a lipid component selected from the group consisting of phosphatidylcholine, phosphatidylglycerol and cholesterol.

According to an embodiment, the invention concerns the vaccine composition, wherein said antigen is conjugated to a carrier protein.

According to an embodiment, the invention concerns the vaccine composition, wherein said antigen is covalently conjugated to a carrier protein.

According to an embodiment, the invention concerns the vaccine composition, wherein said carrier protein is an immunogenic carrier protein.

According to an embodiment, the invention concerns the vaccine composition, wherein said carrier protein is selected from the group consisting of bovine serum albumin (BSA) and keyhole limpet haemocyanin (KLH).

According to an embodiment, the invention concerns the vaccine composition, wherein said vaccine is a multivalent vaccine.

According to an embodiment, the invention concerns the vaccine composition further comprising one or more pharmaceutically acceptable excipients.

According to an embodiment, the invention concerns the vaccine composition further comprising an adjuvant.

According to an embodiment, the invention concerns the vaccine composition, wherein said adjuvant is β-glucan.

According to an embodiment, the invention concerns the vaccine composition, wherein said vaccine is for an administration form selected among subcutaneous, intradermal, intramuscular, intravenous, oral and nasal.

According to an embodiment, the invention concerns the vaccine composition, wherein said vaccine is used for prevention, treatment and/or alleviation of Lyme disease.

According to an embodiment, the invention concerns the vaccine composition, wherein said vaccine is for prevention, treatment and/or alleviation of a condition related to and/or caused by Lyme disease.

According to an embodiment, the invention concerns the vaccine composition, wherein said condition is selected among Lyme Borreliosis, antibiotic resistant Lyme Borreliosis, synovitis, arthritis, antibiotic refractory arthritis, fatigue, sleep impairment, nerve pain, headaches, memory loss, joint pain and/or depression.

According to an embodiment, the invention concerns a method for prevention, treatment and/or alleviation of Lyme disease by administering the vaccine according to the invention to a subject in need thereof.

According to an embodiment, the invention concerns the method, wherein said vaccine is administered in an administration form selected among subcutaneous, intradermal, intramuscular, intravenous, oral and nasal.

According to an embodiment, the invention concerns the method, wherein said vaccine is administered in an administration form selected among oral and nasal.

According to an embodiment the vaccine is prepared as a liposome, comprising one or more lipid antigens embedded in the outer membrane of the liposome(s).

Production of liposomes and encapsulation of compounds in liposomes have been described in the literature e.g. in Henriksen-Lacey et al (2011) Expert Opin. Drug Deliv. 8(4): 505-519 and Wong-Baeza et al (2015) J. Immunol. Res. 2015: Article ID 369462.

Further, several methods for preparing liposomes as such or liposomes encapsulating compounds are known in the art and the present invention is not limited to any of these methods.

In general liposomes are formed in an energy-dependent process, where ingredients are mixed and applied to techniques such as sonication, extrusion, or freeze-drying.

A preferred method for generating liposomes is dissolving the components in a volatile solvent, such as chloroform, combining and then drying off the solvent to create a lipid film. Next, the film is rehydrated in an aqueous buffer and the successive rounds of extrusion is performed to generate unilamellar liposomes with the desired mean diameter, e.g. 100-150 nm. For adjuvant encapsulation a preferred method is freeze-thaw cycling with the prepared liposomes.

The skilled person will appreciate that a major part of the bilayer is formed by the polar lipids forming the bilayer structure wherein other component may be integrated.

The polar lipids of the bilayer may in principle be any such polar lipids. Preferred examples include phosphatidylserine (PS), 1,2-dioleoyl-sn-glycero-3-phospho-L-serine (DOPS), distearoyl-sn-glycero-3-phosphocholine (DSPC), distearoyl-sn-glycero-3-phosphoethanolamine (DSPE), DSPE-PEG2000, distearoyl-sn-glycero-3-phospho-(1′-rac-glycerol) (DSPG), di-(9Z-octadecenoyl)-sn-glycero-3-phosphocholine (DOPC), dihexadecanoyl-sn-glycero-3-phosphocholine (DPPC), Sphingomyelin, Dioleoyl-sn-glycero-3-phosphoethanolamine (DOPE), and steroids such as cholesterol, phytosterol and ergosterol.

According to an embodiment, the liposome comprises DSPC, DSPG, cholesterol and one or more lipid antigens.

Preferred liposomes for use as vaccines according to the invention includes:
Composition A: DSPC/DSPG/Cholesterol/BBGL-1 in the weight ratio: 7:2:1:1;
Composition B: DSPC/DSPG/Cholesterol/BBGL-2 in the weight ratio: 7:2:1:1;
And Composition C: DSPC/DSPG/Cholesterol/BBGL-1/BBGL-2 in the weight ratio: 7:2:1:1:1.

The skilled person will appreciate that the properties of the liposomes will depend on the lipids used for the liposomes and will be capable of selecting a suitable composition of the lipids based on knowledge of lipid chemistry and routine experimentation.

In addition to lipids, the bilayers of the liposome may further comprise additional components including but not limited to antigens, proteins, such as the protein carrier of the invention, stabilizers, and antioxidants.

According to one aspect of the invention, the liposome further comprises a vaccine adjuvant and/or an immunostimulant encapsuled in the lumen of the liposome.

An adjuvant refers to a substance that increases and/or modulates the immune response to a vaccine. The word “adjuvant” comes from the Latin word adiuvare, meaning to help or aid. An immunologic adjuvant refers to any substance that acts to accelerate, prolong, or enhance antigen-specific immune responses when used in combination with specific vaccine antigens.

Immunostimulants, also known as immunostimulators, are substances (drugs and nutrients) that stimulate the immune system by inducing activation or increasing activity of any of its components. One notable example is the granulocyte macrophage colony-stimulating factor.

The vaccine adjuvant and/or immunostimulant may be any such compound or mixture of compounds known in the art to be able to provide vaccine adjuvant function or capable of stimulating an immune response.

Examples of vaccine adjuvants and/or immunostimulants include liposomes themselves, β -glucans, in particular β - 1,3 - D - glucans; aluminum based mineral salts and oil emulsions, saponins such as QS-21, Monophosphoryl lipid A, Cytosine phosphoguanine (CpG), and adjuvant systems such as AS01, which are combinations of adjuvants.

The β-glucan for use as immunostimulant according to the invention may comprise a linear backbone of β-1,3-linked glucose units and may comprise branches of β-1,3-linked glucose units connected to the backbone via β-1,6-linkages. The β-glucan may further comprise β-1,4-linked glucose units, preferably in a number equal to or less that the number of β-1,3-linked glucose units. The β-glucan may additionally comprise one or more charged groups affecting the solubility of the glucan, e.g. selected among charged groups such as phosphate, sulphate, amine and carboxymethyl groups. The β-glucan may in addition to the glucosyl residues comprise additional non-glucose units in amounts less than the amounts of glucose units.

The β-glucan preferably has a molecular weight in the range of 1000 - 100000 Da, preferably in the range of 2000 - 80000 Da, preferably in the range of 3000 - 75000 Da, preferably in the range of 4000 - 60000 Da, preferably in the range of 5000 - 50000 Da.

In a preferred embodiment the β-glucan is derived from a microbial source, e.g. derived from a fungal source such as yeast and mushrooms. For example, may β-glucan for use according to the invention be derived from yeast β-glucan, laminarin, lentinan or schizophyllan; by known procedures for degrading, modifying and/or fractionating carbohydrates.

Particularly preferred is β-glucan comprising a β-1,3 backbone and at least one β-1,3 side chain of two or more glucose units linked to the backbone by β-1,6 glycosidic bonds, and wherein the β-glucan has a molecular weight from about 6 kDa to about 30 kDa, as disclosed in US 8,323,644 B2.

### Synthesis of Lipid Antigens and Derivatives Thereof

The lipid antigens BBGL1, BBGL2 and derivatives thereof, may be prepared by organic chemical synthesis.

BBGL1 may be synthesized as illustrated in the reaction scheme in FIG. 8:

The synthesis is illustrated using a Benzoyl group as an alcohol protective group, but the skilled person will appreciate that the reaction is not limited to this used protective group but may be performed using other alcohol protective groups, as known in the art.

BBGL1-Biotin may be synthesized as illustrated in the reaction scheme in FIG. 9:

The synthesis is illustrated using Fmoc (9H-Fluoren-9-carbonyl) as an amine protective group, but the skilled person will appreciate that the reaction is not limited to this used protective group, but may be performed using other amine protective groups, as known in the art.

The compounds 6-[12-(X-amino)-dodecanoyl]-1-cholesteryl-β-D-galactopyranoside, where X is an amino protective group; 6-[12-(9H-Fluoren-9-ylmethoxycarbonylamino)-dodecanoyl]-1-cholesteryl-β-D-galactopyranoside (compound 7 in the reaction scheme above) and BBGL1-Biotin are new compounds, and these compounds forms additional embodiments of the invention.

BBGL2 may be synthesized as illustrated in the reaction scheme in FIG. 10:

The synthesis is illustrated using a methoxyacetyl group as an alcohol protective group, but the skilled person will appreciate that the reaction is not limited to this used protective group but may be performed using other alcohol protective groups, as known in the art.

The compounds Allyl 2,3,4,6-tetra-O-X1-α-D-galactopyranoside, Allyl 2,3,4,6-tetra-O-methoxyacetyl-α-D-galactopyranoside (compound 9 in the reaction scheme above), (2′R)-2′,3′-Epoxypropyl 2,3,4,6-tetra-O-X1-α-D-galactopyranoside, (2′R)-2′,3′-Epoxypropyl 2,3,4,6-tetra-O-methoxyacetyl-α-D-galactopyranoside ( compound 10 in the reaction scheme above), (2′R)-3′-Bromo-2′-hydroxypropyl 2,3,4,6-tetra-O-X1-α-D-galactopyranoside, (2′R)-3′-Bromo-2′-hydroxypropyl 2,3,4,6-tetra-O-methoxyacetyl-α-D-galactopyranoside (compound 11 in the reaction scheme above) (2′R)-3′-Bromo-2′-palmitoyloxypropyl 2,3,4,6-tetra-O-X1-α-D-galactopyranoside, (2′R)-3′-Bromo-2′-palmitoyloxypropyl 2,3,4,6-tetra-O-methoxyacetyl-α-D-galactopyranoside (compound 12 in the reaction scheme above), 1-O-oleoyl-2-O-palmitoyl-sn-glyceryl 2,3,4,6-tetra-O-X1-α-D-galactopyranoside and 1-O-oleoyl-2-O-palmitoyl-sn-glyceryl 2,3,4,6-tetra-O-methoxyacetyl-α-D-galactopyranoside (compound 13 in the reaction scheme above), wherein X1 is an alcohol protection group, are new compounds and these compounds forms additional embodiments of the invention.

BBL2-Biotin may be synthesized as illustrated in the reaction scheme in FIG. 11:

The synthesis is illustrated using Fmoc (9H-Fluoren-9-carbonyl) as an amine protective group, and Methoxyacelyl as alcohol protective group, but the skilled person will appreciate that the reaction is not limited to this particular used protective group, but may be performed using other protective groups, as known in the art.

The compounds (2′R)-3′-Bromo-2′-[12-(X2-amino)-dodecanoyl]-2,3,4,6-tetra-O-X3-α-D-galactopyranoside, (2′R)-3′-Bromo-2′-[12-(9H-Fluoren-9-ylmethoxycarbonylamino)-dodecanoyl]-2,3,4,6-tetra-O-methoxyacetyl-α-D-galactopyranoside (compound 14 in the reaction scheme above), (2′R)-3′-Bromo-2′-[12-biotinylamino)-dodecanoyl]-2,3,4,6-tetra-O-X3-α-D-galactopyranoside, (2′R)-3′-Bromo-2′-[12-biotinylamino)-dodecanoyl]-2,3,4,6-tetra-O-methoxyacetyl-α-D-galactopyranoside (compound 15 in the reaction scheme above), (2′R)-3′-oleoyl-2′-[12-biotinylamino)-dodecanoyl]-2,3,4,6-tetra-O-X3-α-D-galactopyranoside, (2′R)-3′-oleoyl-2′-[12-biotinylamino)-dodecanoyl]-2,3,4,6-tetra-O-methoxyacetyl-α-D-galactopyranoside (compound 16 in the reaction scheme above), wherein X2 is an amine protective group and X3 is an alcohol protective group, and BBGL2-Biotin are new com pounds and these compounds form additional embodiments of the invention.

Certain embodiments of the invention relate to antibodies.

According to an embodiment, the invention concerns an antibody or fragment thereof binding a lipid antigen.

According to an embodiment, the invention concerns the antibody or fragment thereof, wherein said lipid antigen is a glycolipid antigen.

According to an embodiment, the invention concerns the antibody or fragment thereof, wherein said lipid antigen is a BBGL-1 and/or BBGL-2 antigen.

According to an embodiment, the invention concerns the antibody or fragment thereof binding a BBGL-1 antigen.

According to an embodiment, the invention concerns the antibody or fragment thereof binding a BBGL-2 antigen.

According to an embodiment, the invention concerns the antibody or fragment thereof binding a BBGL-1 antigen and a BBGL-2 antigen.

According to an embodiment, the invention concerns the antibody or fragment thereof, wherein said antibody or fragment thereof comprises a homodimeric immunoglobulin or fragment thereof.

According to an embodiment, the invention concerns the antibody or fragment thereof, wherein said antibody or fragment thereof comprises a heterodimeric immunoglobulin or fragment thereof.

According to an embodiment, the invention concerns the antibody or fragment thereof, wherein said antibody or fragment thereof is an IgG, IgM IgA, IgD and/or an IgE.

According to an embodiment, the invention concerns the antibody or fragment thereof, wherein said antibody or fragment thereof is an IgG1, IgG3 and/or IgM.

According to an embodiment, the invention concerns the antibody or fragment thereof, wherein said antibody or fragment thereof comprises a sequence selected from the group consisting of SEQ ID NOs 1-20.

According to an embodiment, the invention concerns the antibody or fragment thereof, wherein said antibody or fragment thereof comprises a sequence combination selected from the specific combinations given in Table 3.

According to an embodiment, the invention concerns a method of producing an antibody or fragment thereof according to the invention, wherein said antibody or fragment thereof is produced by animal immunization.

According to an embodiment, the invention concerns the method, wherein said animal immunization comprises administering a vaccine according to the invention to an animal.

According to an embodiment, the invention concerns the method, wherein said animal is selected from the group consisting of mouse, rabbit, alpaca and llama.

According to an embodiment, the invention concerns a method of producing an antibody or fragment thereof according to the invention comprising
a. Isolating B cells from a Lyme diseases patient,
b. Isolating B-cell receptors from said B cells,
c. Sequencing said B-cell receptors, and
d. Cloning said B-cell receptors.

According to an embodiment, the invention concerns a method of producing an antibody or fragment thereof according to the invention using a human antibody phage display library.

According to an embodiment, the invention concerns an antibody or fragment thereof obtainable by the method of the invention.

According to an embodiment, the invention concerns the antibody or fragment thereof, wherein said antibody or fragment thereof further binds a CD3 antigen.

According to an embodiment, the invention concerns the antibody or fragment thereof, wherein said antibody or fragment thereof is a T cell engaging antibody.

According to an embodiment, the invention concerns the antibody or fragment thereof, wherein said antibody or fragment thereof comprises variable light chain CDR sequences of SEQ ID NOs:75-77.

According to an embodiment, the invention concerns the antibody or fragment thereof, wherein said antibody or fragment thereof comprises variable heavy chain CDR sequences of SEQ ID NOs:78-80.

According to an embodiment, the invention concerns the antibody or fragment thereof, wherein said antibody or fragment thereof comprises variable light chain CDR sequences of SEQ ID NOs:75-77 and variable heavy chain CDR sequences of SEQ ID NOs:78-80.

According to an embodiment, the invention concerns the antibody or fragment thereof, wherein said antibody or fragment thereof comprises a sequence selected from the group consisting of SEQ ID NOs 21-38.

According to an embodiment, the invention concerns the antibody or fragment thereof, wherein said antibody or fragment thereof comprises a sequence selected from the group consisting of SEQ ID NOs 39-57.

According to an embodiment, the invention concerns the antibody or fragment thereof, wherein said antibody or fragment thereof comprises a sequence combination selected from the specific combinations given in Table 4.

According to an embodiment, the invention concerns the antibody or fragment thereof, wherein said antibody or fragment thereof further binds a TCR Va24Ja18 antigen.

According to an embodiment, the invention concerns the antibody or fragment thereof, wherein said TCR Va24Ja18 antigen comprises a sequence according to SEQ ID No.: 81, 82 and/or 83.

According to an embodiment, the invention concerns the antibody or fragment thereof, wherein said antibody is an NKT cell engaging antibody.

According to an embodiment, the invention concerns the antibody or fragment thereof, wherein said antibody or fragment thereof comprises a variable light chain sequence selected from the group consisting of SEQ ID NOs 58-60.

According to an embodiment, the invention concerns the antibody or fragment thereof, wherein said antibody or fragment thereof comprises a variable heavy chain sequence selected from the group consisting of SEQ ID NOs 61-63.

According to an embodiment, the invention concerns the antibody or fragment thereof, wherein said antibody or fragment thereof comprises a variable light chain sequence selected from the group consisting of SEQ ID NOs 58-60 and a variable heavy chain sequence selected from the group consisting of SEQ ID NOs 61-63.

According to an embodiment, the invention concerns the antibody or fragment thereof, wherein said antibody or fragment thereof comprises a sequence selected from the group consisting of SEQ ID NOs 64-69.

According to an embodiment, the invention concerns the antibody or fragment thereof, wherein said antibody or fragment thereof comprises a sequence combination selected from the specific combinations given in Table 10.

According to an embodiment, the invention concerns the antibody or fragment thereof, wherein said antibody or fragment thereof further binds a CD16 and/or a CD56 antigen.

According to an embodiment, the invention concerns the antibody or fragment thereof, wherein said antibody or fragment thereof further binds a CD16 and a CD56 antigen.

According to an embodiment, the invention concerns the antibody or fragment thereof, wherein said antibody or fragment thereof specifically activates NKT cells.

According to an embodiment, the invention concerns the antibody or fragment thereof, wherein said antibody or fragment thereof comprises a sequence combination selected from the specific combinations given in Table 11.

According to an embodiment, the invention concerns the antibody or fragment thereof, wherein said antibody or fragment thereof engage a cell selected from the group consisting of T cells, NK cells and NKT cells.

According to an embodiment, the invention concerns the antibody or fragment thereof, wherein said antibody or fragment thereof further comprises a drug payload.

According to an embodiment, the invention concerns the antibody or fragment thereof, wherein said drug payload is an infrared dye and/or a radionucleotide.

According to an embodiment, the invention concerns the antibody or fragment thereof, wherein said antibody or fragment thereof comprises a mutation selected from the group consisting of an Ala339Cys, a Ser337Cys and a Lys340Cys mutation.

According to an embodiment, the invention concerns the antibody or fragment thereof, wherein said antibody or fragment thereof comprises an Ala339Cys mutation.

According to an embodiment, the invention concerns the antibody or fragment thereof, wherein said drug payload is inserted at a site-specific free Cys residue.

According to an embodiment, the invention concerns the antibody or fragment thereof, wherein said antibody or fragment thereof comprises a sequence selected from the group consisting of SEQ ID NOs 70-73.

According to an embodiment, the invention concerns the antibody or fragment thereof, wherein said antibody or fragment thereof comprises a sequence combination selected from the specific combinations given in Table 13.

According to an embodiment, the invention concerns the antibody or fragment thereof, wherein said antibody or fragment thereof is a scFv.

According to an embodiment, the invention concerns the antibody or fragment thereof, wherein said antibody or fragment thereof comprises a scFv.

According to an embodiment, the invention concerns the antibody or fragment thereof, wherein said antibody or fragment thereof provides enhanced ADCC, ADCP and/or CDC.

According to an embodiment, the invention concerns the antibody or fragment thereof comprising a first antigen binding site capable of binding to a first antigen and a second antigen binding site capable of binding to a second antigen, wherein said first antigen binding site is comprised in a Fab fragment and said second antigen binding site is comprised in a moiety selected from the group consisting of scFv, antibody fragment and protein moiety, characterized by said moiety being attached to the light chain of said Fab fragment.

According to an embodiment, the invention concerns the antibody or fragment thereof, wherein said moiety is a scFv.

According to an embodiment, the invention concerns the antibody or fragment thereof, wherein said moiety is attached to the C-terminus or N-terminus of the light chain of said Fab fragment.

According to an embodiment, the invention concerns the antibody or fragment thereof, wherein said moiety is attached to the C-terminus of the light chain of said Fab fragment.

According to an embodiment, the invention concerns the antibody or fragment thereof, wherein the constant region is derived from an IgG or an IgM.

According to an embodiment, the invention concerns the antibody or fragment thereof, wherein said antibody or fragment thereof comprises a mutation in the IgM constant region, preferably at position 131, 135, 354 and/or 385.

According to an embodiment, the invention concerns the antibody or fragment thereof, wherein said antibody or fragment thereof comprises a mutation in the IgM constant region selected from the group consisting of K131R, K131H, K131D, K131E, Q135K, Q135R, Q135H, Q135D, Q135E, T354D, T354E, T354K, T354R, T354H, E385D, E385K, E385R and E385H.

According to an embodiment, the invention concerns the antibody or fragment thereof, wherein said Fab fragment is derived from an IgG.

According to an embodiment, the invention concerns the antibody or fragment thereof, wherein said Fab fragment is derived from an IgG selected from the group consisting of IgG1, IgG2, IgG3 and IgG4.

According to an embodiment, the invention concerns the antibody or fragment thereof, wherein said Fab fragment is derived from an IgG2 or an IgG4.

According to an embodiment, the invention concerns the antibody or fragment thereof, wherein said antibody or fragment thereof comprises a null fc.

According to an embodiment, the invention concerns the antibody or fragment thereof, wherein said antibody or fragment thereof comprises a null fc, wherein said null fc comprises a Leu234Ala and/or a Leu235Ala and/or a Lys22Ala mutation.

According to an embodiment, the invention concerns the antibody or fragment thereof, wherein said antibody or fragment thereof comprises a Ser228Pro mutation.

According to an embodiment, the invention concerns the antibody or fragment thereof comprising a linker.

According to an embodiment, the invention concerns the antibody or fragment thereof, wherein said linker is a peptide linker.

According to an embodiment, the invention concerns the antibody or fragment thereof, wherein said linker is a GlySer linker.

According to an embodiment, the invention concerns the antibody or fragment thereof, wherein said linker comprises the sequence of SEQ ID NO:92.

According to an embodiment, the invention concerns the antibody or fragment thereof, wherein said antibody or fragment thereof blocks host cell entry of a pathogen.

Certain embodiments of the invention relate to pharmaceutical compositions comprising an antibody.

According to an embodiment, the invention concerns a pharmaceutical composition comprising an antibody or fragment thereof according to the invention, optionally comprising one or more excipients such as diluents, binders or carriers.

According to an embodiment, the invention concerns the pharmaceutical composition further comprising an adjuvant.

According to an embodiment, the invention concerns the pharmaceutical composition, wherein said adjuvant is β-glucan.

According to an embodiment, the invention concerns the antibody or fragment thereof or pharmaceutical composition, wherein said antibody or fragment thereof or pharmaceutical composition is for an administration form selected among subcutaneous, intradermal, intramuscular, intravenous, oral and nasal.

Certain embodiments of the invention relate to nucleic acids and methods of producing said antibody.

According to an embodiment, the invention concerns an isolated nucleic acid molecule encoding an antibody or fragment thereof according to the precedent invention.

According to an embodiment, the invention concerns a recombinant vector comprising the nucleic acid molecule of the invention.

According to an embodiment, the invention concerns a host cell comprising the recombinant vector of the invention.

According to an embodiment, the invention concerns a method to produce an antibody or fragment thereof according to the invention comprising a step of culturing the host cell according to the invention in a culture medium under conditions allowing the expression of the antibody or fragment thereof and separating the antibody or fragment thereof from the culture medium.

According to an embodiment, the invention concerns a method to produce an antibody or fragment thereof according to the invention comprising a synthetic and/or recombinant step.

Certain embodiments of the invention relate to treatment methods.

According to an embodiment, the invention concerns use of an antibody or fragment thereof or vaccine composition according to the invention in a preclinical model.

According to an embodiment, the invention concerns a method of preventing, treating and/or alleviating an infectious disease comprising administering to a patient in need thereof a therapeutically effective amount of the antibody or fragment thereof or pharmaceutical composition according to the invention.

According to an embodiment, the invention concerns the method, wherein said infectious disease is caused by a Borrelia Burgdorferi bacteria sensu lato.

According to an embodiment, the invention concerns the method, wherein said Borrelia Burgdorferi bacteria sensu lato is B. burgdorferi, B. mayonii, B. afzelii and/or B. garinii.

According to an embodiment, the invention concerns a method of preventing, treating and/or alleviating a condition related to and/or caused by Lyme disease comprising administration to a patient in need thereof a therapeutically effective amount of the antibody or fragment thereof or pharmaceutical composition according to the invention.

According to an embodiment, the invention concerns the method, wherein condition is selected among Lyme Borreliosis, antibiotic resistant Lyme Borreliosis, synovitis, arthritis, antibiotic refractory arthritis, fatigue, sleep impairment, nerve pain, headaches, memory loss, joint pain and/or depression.

According to an embodiment, the invention concerns a method of preventing, treating and/or alleviating Lyme disease comprising administering to a patient in need thereof a therapeutically effective amount of the antibody or fragment thereof or pharmaceutical composition according to the invention.

Certain embodiments of the invention relate to diagnostics.

According to an embodiment, the invention concerns a method of diagnosing Lyme disease comprising administering to a patient in need thereof a therapeutically effective amount of the antibody or fragment thereof or pharmaceutical composition according to the invention.

According to an embodiment, the invention concerns a method of diagnosing Lyme disease comprising the steps of
a. obtaining a blood or urine sample from a subject, and
b. testing said blood or urine sample for the presence of a BBGL-1 and/or BBGL-2 biomarker with the antibody or fragment thereof according to the invention.

According to an embodiment, the invention concerns a method of selecting and/or identifying a subject that might benefit from treatment with an antibody or fragment thereof or vaccine according to the invention comprising the steps of
a. obtaining a blood or urine sample from a subject, and
b. testing said blood or urine sample for the presence of a BBGL-1 and/or BBGL-2 biomarker with the antibody or fragment thereof according to the invention.

According to an embodiment, the invention concerns a method of monitoring infectious disease progression and/or disease resolution comprising the steps of
a. obtaining a blood or urine sample from a subject, and
b. testing said blood or urine sample for the presence of a BBGL-1 and/or BBGL-2 biomarker with the antibody or fragment thereof according to the invention.

BBGL1-biotin is a preferred biomarker for use in the invention, because it contains the lipid antigen BBGL-1 and a biotin group that easily can be detected by binding to streptavidin.

BBGL2-biotin is a preferred biomarker for use in the invention, because it contains the lipid antigen BBGL-2 and a biotin group that easily can be detected by binding to streptavidin.

As well known in the art, biotin binds streptavidin with high affinity and this has been used in a vast amount of detection/binding application in the art. The skilled person will appreciate that any such tools developed in the prior art that utilizes biotin -streptavidin binding may be adapted to and used with the lipid antigens BBGL1-Biotin and BBGL2-Biotin.

According to an embodiment, the invention concerns a method of in vivo imaging of bacterial infections comprising an antibody or fragment thereof according to the invention.

According to an embodiment, the invention concerns the antibody or fragment thereof for use in an imaging technique for monitoring infectious disease progression/resolution.

According to an embodiment, the invention concerns a diagnostic kit comprising an antibody or fragment thereof according to the invention and instructions for use.

According to an embodiment, the invention concerns a kit for screening comprising an antibody or fragment thereof according to the invention and instructions for use.

According to an embodiment, the invention concerns an antibody or fragment thereof, preferably according to the invention, capable of binding a TCR Va24Ja18 antigen, wherein said antibody is an NKT cell engaging antibody; and preferably wherein said antibody or fragment thereof is capable of binding an additional antigen, such as a humanized 6B11 antigen, preferably according to the description.

According to an embodiment, the invention concerns an antibody or fragment thereof, preferably according to the invention, capable of binding a CD3 antigen, and wherein said antibody or fragment further is capable of binding a CD16 and/or a CD56 antigen; and preferably wherein said antibody or fragment thereof is capable of binding an additional antigen allowing NKT cells to be re-directed to said additional antigen.

According to an embodiment, the invention concerns an antibody or fragment thereof, capable of binding a TCR Va24Ja18 antigen, wherein said antibody is an NKT cell engaging antibody,
wherein said antibody or fragment thereof further binds a CD3 antigen, and
wherein said antibody or fragment thereof further binds a CD16 and/or a CD56 antigen.

According to an embodiment, the invention concerns the antibody or fragment thereof, wherein said TCR Va24Ja18 antigen comprises a sequence according to SEQ ID NO.: 81, 82 and/or 83.

According to an embodiment, the invention concerns the antibody or fragment thereof, wherein said antibody or fragment thereof comprises
a. a variable light chain sequence comprising at least one of the CDR regions according to SEQ ID No.: 84 - 86.
b. A variable heavy chain sequence comprising at least one of the CDR regions according to SEQ ID No.: 87 - 89.

According to an embodiment, the invention concerns the antibody or fragment thereof, wherein said CD3 antigen and said CD16 and/or a CD56 antigen are expressed on NKT cells.

According to an embodiment, the invention concerns the antibody or fragment thereof, wherein said antibody or fragment thereof is capable of binding to said CD3 antigen and simultaneously to said CD16 and/or CD56 antigen.

According to an embodiment, the invention concerns a compound, such as a compound selected among the group of compounds disclosed in the experimental section.

According to an embodiment, the invention concerns a compound selected among the group consisting of 6-[12-(X-amino)-dodecanoyl]-1-cholesteryl-β-D-galactopyranoside, where X is an amino protective group; 6-[12-(9H-Fluoren-9-ylmethoxycarbonylamino)-dodecanoyl]-1-cholesteryl-β-D-galactopyranoside (compound 7 in the reaction scheme) and BBGL1-Biotin.

According to an embodiment, the invention concerns a compound selected among the group consisting of Allyl 2,3,4,6-tetra-O-X1-α-D-galactopyranoside, Allyl 2,3,4,6-tetra-O-methoxyacetyl-α-D-galactopyranoside (compound 9 in the reaction scheme), (2′R)-2′,3′-Epoxypropyl 2,3,4,6-tetra-O-X1-α-D-galactopyranoside, (2′R)-2′,3′-Epoxypropyl 2,3,4,6-tetra-O-methoxyacetyl-a-D-galactopyranoside (compound 10 in the reaction scheme), (2′R)-3′-Bromo-2′-hydroxypropyl 2,3,4,6-tetra-O-X1-α-D-galactopyranoside, (2′R)-3′-Bromo-2′-hydroxypropyl 2,3,4,6-tetra-O-methoxyacetyl-α-D-galactopyranoside (compound 11 in the reaction scheme) (2′R)-3′-Bromo-2′-palmitoyloxypropyl 2,3,4,6-tetra-O-X1-α-D-galactopyranoside, (2′R)-3′-Bromo-2′-palmitoyloxypropyl 2,3,4,6-tetra-O-methoxyacetyl-α-D-galactopyranoside (compound 12 in the reaction scheme), 1-O-oleoyl-2-O-palmitoyl-sn-glyceryl 2,3,4,6-tetra-O-X1-α-D-galactopyranoside and 1-O-oleoyl-2-O-palmitoyl-sn-glyceryl 2,3,4,6-tetra-O-methoxyacetyl-α-D-galactopyranoside (compound 13 in the reaction scheme), wherein X1 is an alcohol protection group.

According to an embodiment, the invention concerns a compound selected among the group consisting of (2′R)-3′-Bromo-2′-[12-(X2-amino)-dodecanoyl]-2,3,4,6-tetra-O-X3-α-D-galactopyranoside, (2′R)-3′-Bromo-2′-[12-(9H-Fluoren-9-ylmethoxycarbonylamino)-dodecanoyl]-2,3,4,6-tetra-O-methoxyacetyl-a-D-galactopyranoside (compound 14 in the reaction scheme), (2′R)-3′-Bromo-2′-[12-biotinylamino)-dodecanoyl]-2,3,4,6-tetra-O-X3-α-D-galactopyranoside, (2′R)-3′-Bromo-2′-[12-biotinylamino)-dodecanoyl]-2,3,4,6-tetra-O-methoxyacetyl-α-D-galactopyranoside (compound 15 in the reaction scheme), (2′R)-3′-oleoyl-2′-[12-biotinylamino)-dodecanoyl]-2,3,4,6-tetra-O-X3-α-D-galactopyranoside, (2′R)-3′-oleoyl-2′-[12-biotinylamino)-dodecanoyl]-2,3,4,6-tetra-O-methoxyacetyl-α-D-galactopyranoside (compound 16 in the reaction scheme), wherein X2 is an amine protective group and X3 is an alcohol protective group, and BBGL2-Biotin.

Immunoglobulins are glycoproteins composed of one or more units, each containing four polypeptide chains: two identical heavy chains (HCs) and two identical light chains (LCs). The amino terminal ends of the polypeptide chains show considerable variation in amino acid composition and are referred to as the variable (V) regions to distinguish them from the relatively constant (C) regions. Each light chain consists of one variable domain, VL, and one constant domain, CL. The heavy chains consist of a variable domain, VH, and three constant domains CH1, CH2 and CH3. Heavy and light chains are held together by a combination of non-covalent interactions and covalent interchain disulfide bonds, forming a bilaterally symmetric structure. The V regions of H and L chains comprise the antigen-binding sites of the immunoglobulin (Ig) molecules. Each Ig monomer contains two antigen-binding sites and is said to be bivalent.

The Fab contains one complete L chain in its entirety and the V and CH1 portion of one H chain. The Fab can be further divided into a variable fragment (Fv) composed of the VH and VL domains, and a constant fragment (Fb) composed of the CL and CH1 domains.

The H chain constant domain is generally defined as CH1-CH2-CH3 (IgG, IgA, IgD) with an additional domain (CH4) for IgM and IgE. As described above, the CH1 domain is located within the F(ab) region whereas the remaining CH domains (CH2-CH3 or CH2-CH4) comprise the Fc fragment. This Fc fragment defines the isotype and subclass of the immunoglobulin.

CH3 domain: The terms CH3 domain and CH3 region are used interchangeable herein.

CH1 domain: The terms CH1 domain and CH1 region are used interchangeable herein.

Hinge region: The hinge region is the area of the heavy chains between the first and second C region domains and is held together by disulfide bonds. A hinge region typically comprises between 10 and 30 amino acid residues.

Linker: A linker might be a peptide linker or a non-peptide linker. An example of a peptide linker is a Gly/Ser peptide linker comprising a five amino acid residue unit, GGGGS (SEQ ID NO:92), that can be repeated a suitable number of times. A linker might be a naturally occurring linker or a synthetically produced linker. A linker might occur naturally in a molecule or might be synthetically added to a molecule.

Antibody fragment: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region. An antigen-binding fragment of an antibody may be produced by any means. For example, an antigen-binding fragment of an antibody may be enzymatically or chemically produced by fragmentation of an intact antibody and/or it may be recombinantly produced from a gene encoding the partial antibody sequence. Alternatively, or additionally, antigen-binding fragment of an antibody may be wholly or partially synthetically produced. An antigen-binding fragment of an antibody may optionally comprise a single chain antibody fragment. Alternatively, or additionally, an antigen-binding fragment of an antibody may comprise multiple chains that are linked together, for example, by disulfide linkages. An antigen-binding fragment of an antibody may optionally comprise a multi-molecular complex. A functional antibody fragment typically comprises at least about 50 amino acids and more typically comprises at least about 200 amino acids.

Antibody or fragment thereof: As used herein, an “antibody or fragment thereof” refers to an antibody or antibody fragment as defined above.

Humanized antibodies: Humanized antibodies are antibodies from non-human species whose protein sequences have been modified to increase their similarity to antibody variants produced naturally in humans.

IMGT: the international ImMunoGeneTics information system, is an international reference in immunogenetics and immunoinformatics.

Single-chain Fv (scFv): Single-chain Fvs (scFvs) are widely known and used in the art. A single-chain Fv is a fusion protein of the variable regions of the heavy (VH) and light chains (VL) of immunoglobulins, often connected by a short linker peptide (see, e.g., see, e.g., Benny K. C. Lo (ed.), Antibody Engineering - Methods and Protocols, Humana Press 2004, and references cited therein).

Adjuvants: Adjuvants can potentiate the immunogenicity of antigens and modulate the immune response (Petrovsky 2015). Aluminum is a neurotoxin and nevertheless aluminum salts are the most commonly used vaccine adjuvants (Tomljenovic 2011). Glycolipid adjuvants such as galactosylceramides that are extracted from marine sponges, are potent NK T cell stimulators that have been used to stimulate immune responses to malaria and cancer vaccines however some studies suggest adjuvant related hepatotoxicity (Gonzalez-Aseguinolaza, 2010, Ko 2005, Tefit 2014). B-glucan is a GRAS (generally recognized as safe) ingredient and a promising polysaccharide oral vaccine adjuvant that stimulates various immune reactions including antibody production without no negative side effects. B-glucan has been successfully used in combination with a GD2/GD3 cancer vaccine for pediatric neuroblastoma (NCT00911560, Kushner 2014).

A lipid antigen might be defined as a lipid molecule or lipid molecular structure that may be presented at the outside of a pathogen, and which induces an immune response in a subject.

Homodimeric antibodies may be defined as an antibody formed by two identical proteins.

Heterodimeric antibodies may be defined as an antibody formed by two different proteins.

CD16 may also be referred to as FcγRIII. CD16 may be defined as a cluster of differentiation molecule and may be found on the surface of natural killer cells.

CD56 may also be referred to as Neural cell adhesion molecule (NCAM) and may be expressed by natural killer cells.

### FIGURES

FIG. 1 shows two Lyme Disease antigens BBGL-1 (cholesteryl 6-O-palmitoyl-β-D-galactopyranoside) and BBGL-2 (1-oleoyl-2-palmitoyl-3-(α-D-galactosyl)-sn-glycerol).

FIG. 2 shows three vaccine constructs containing either anti-BBGL-1, anti-BBGL-2 or the combination of anti-BBGL-1 and anti-BBGL-2.

FIG. 3 shows anti-BBGL-1 and anti-BBGL-2 antibodies.

FIG. 4 shows anti-BBGL-1 and anti-BBGL-2 bispecific antibodies with T cell engager effector functions.

FIG. 5 shows anti-BBGL-1 and anti-BBGL-2 bispecific antibodies with NKT cell engager effector functions.

FIG. 6 shows anti-BBGL-1 and anti-BBGL-2 multi-specific antibodies with T cell, NK cell, and NKT cell engager effector functions.

FIG. 7a shows in vitro neutralization of Borrelia burgdorferi of Day 30 serum samples from immunized mice (n = 6 mice per group)

FIG. 7b shows the average weight of mice (in grams with standard deviation) per group of immunized mice (12 mice per group) over the 60 day experiment

FIG. 8 Synthesis of BbGL1

FIG. 9 Synthesis of BbGL1-Biotin

FIG. 10 Synthesis of BbGL2

FIG. 11 Synthesis of BbGL2-Biotin

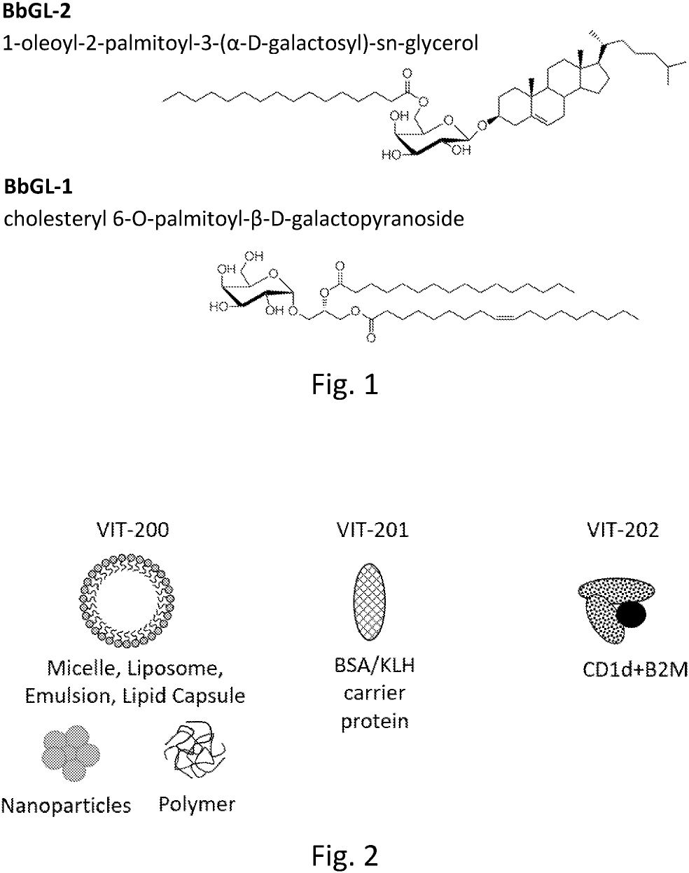

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 8.0.

### EXAMPLES

### Example 1: Design of BBGL-1 and BBGL-2 Vaccines

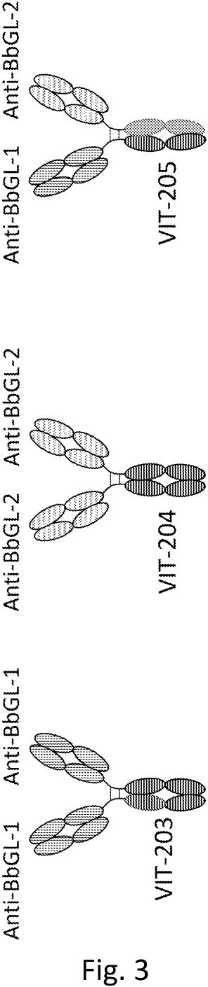

Three vaccine constructs were designed that contain either BBGL-1, BBGL-2 or the combination of BBGL-1 and BBGL-2 (see FIG. 2).

In format VIT-200, BBGL-1 and/or BBGL-2 are reconstituted into liposomes, solid lipid nanoparticles or micelles containing other lipid components, such as phosphatidylcholine, phosphatidylglycerol and cholesterol.

In format VIT-201, BBGL-1 and/or BBGL-2 are covalently conjugated to immunogenic carrier proteins such as BSA (bovine serum albumin) and KLH (keyhole limpet hemocyanin).

In format VIT-202, BBGL-1 and/or BBGL-2 are loaded onto the extracellular domain of recombinant human CD1d and human B2M (Beta-2-microglobulin). This allows for BBGL-1 and/or BBGL-2 to be directly presented to NKT cells.

### Example 2: Design of anti-BBGL-1 and anti-BBGL-2 Antibodies with IgG1, IgG3 or IgM Effector Functions

Anti-BBGL-1 and anti-BBGL-2 antibodies are derived from immunization of animals (mouse, rabbit, llama) with vaccines from Example 1, or from B cell isolation and sequencing from Lyme disease patients.

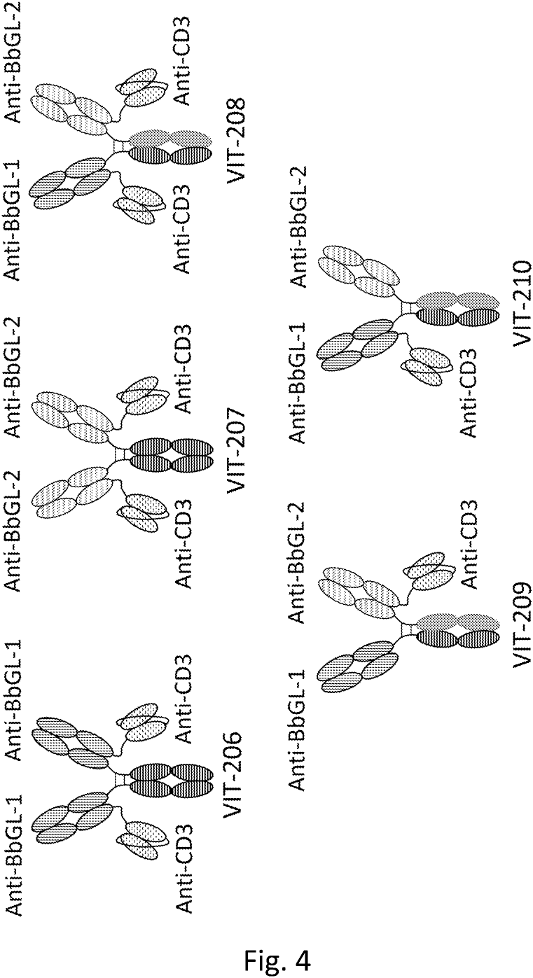

In constructs VIT-203 and VIT-204 (FIG. 3), the anti-BBGL-1 and anti-BBGL-2 antibodies have homodimeric IgG1, IgG3 or IgM effector functions. In construct VIT-205 (FIG. 3), the anti-BBGL-1 x anti-BBGL-2 antibodies have heterodimeric IgG1, IgG3 or IgM effector functions.

In the below tables, the anti-BBGL1 antibodies have the VH sequences denoted as BBGL1-VH, VL-kappa sequences as BBGL1-VLK, and VL-lambda sequences as BBGL1-VLL. The anti-BBGL2 antibodies have the VH sequences denoted as BBGL2-VH, VL-kappa sequences as BBGL2-VLK, and VL-lambda sequences as BBGL2-VLL. The scFv versions of the above constructs will be termed BBGL1-VH-VLK-scFv, BBGL1-VH-VLL-scFv, BBGL2-VH-VKL-scFv, and BBGL2-VH-VLL-scFv.

TABLE 1

Constant regions for IgG1-like constructs

| | | |
|---|---|---|
| Construct | Sequence | Notes |
| WT huIgG1 CH1-CH2-CH3 | ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPELLGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALPAPIEKTISKAKGQPREPQVYTLPPSRDELTKN QVSLTCLVKGFYPSDIAVEWESNGQPENNYKTTPPVLDSD GSFFLYSKLTVDKSRWQQGNVFSCSVMHEALHNHYTQKS LSLSPGK (SEQ ID NO:1) | |
| V-IGG1 | ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPELLGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALPAPIEKTISKAKGQPREPQVYTLPPSRDELTKN QVSLTCLVKGFYPSDIAVEWESNGQPENNYKTTPPVLDSD GSFFLYSKLTVDKSRWQQGNVFSCSVMHEALHNHYTQKS LSLSPGK (SEQ ID NO:2) | Same as above, hinge region underlined |
| V-IGG1-Fc | GGGGSGGGGSGGGGSDKTHTCPPCPAPELLGGPSVFLFPP KPKDTLMISRTPEVTCVVVDVSHEDPEVKFNWYVDGVEV HNAKTKPREEQYNSTYRVVSVLTVLHQDWLNGKEYKCKVS NKALPAPIEKTISKAKGQPREPQVYTLPPSRDELTKNQVSLT CLVKGFYPSDIAVEWESNGQPENNYKTTPPVLDSDGSFFLY SKLTVDKSRWQQGNVFSCSVMHEALHNHYTQKSLSLSPG K (SEQ ID NO:3) | Same as above, but with linker and fc only |
| V-IGG1-A | ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPELLGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALPAPIEKTISKAKGQPREPQVYTLPPSRDELTKN QVSLTCLVKGFYPSDIAVEWESNGQPENNYKTTPPVLDSD GSFFLYSRLTVDKSRWQQGNVFSCSVMHEALHNHYTQKS LSLSPGK (SEQ ID NO:4) | K409R |
| V-IGG1-A-Fc | GGGGSGGGGSGGGGSDKTHTCPPCPAPELLGGPSVFLFPP KPKDTLMISRTPEVTCVVVDVSHEDPEVKFNWYVDGVEV HNAKTKPREEQYNSTYRVVSVLTVLHQDWLNGKEYKCKVS NKALPAPIEKTISKAKGQPREPQVYTLPPSRDELTKNQVSLT CLVKGFYPSDIAVEWESNGQPENNYKTTPPVLDSDGSFFLY | Same as SEQ NO 4, but with linker and fc only |
| | SRLTVDKSRWQQGNVFSCSVMHEALHNHYTQKSLSLSPG K (SEQ ID NO:5) | |
| V-IGG1-B | ASTKGPSVFPLAPSSKSTSGGTAALGCLVKDYFPEPVTVSW NSGALTSGVHTFPAVLQSSGLYSLSSVVTVPSSSLGTQTYIC NVNHKPSNTKVDKKVEPKSCDKTHTCPPCPAPELLGGPSV FLFPPKPKDTLMISRTPEVTCVVVDVSHEDPEVKFNWYVD GVEVHNAKTKPREEQYNSTYRVVSVLTVLHQDWLNGKEY KCKVSNKALPAPIEKTISKAKGQPREPQVYTLPPSRDELTKN QVSLTCLVKGFYPSDIAVEWESNGQPENNYKTTPPVLDSD GSFLLYSKLTVDKSRWQQGNVFSCSVMHEALHNHYTQKS LSLSPGK (SEQ ID NO:6) | F405L |
| V-IGG1-B-Fc | GGGGSGGGGSGGGGSDKTHTCPPCPAPELLGGPSVFLFPP KPKDTLMISRTPEVTCVVVDVSHEDPEVKFNWYVDGVEV HNAKTKPREEQYNSTYRVVSVLTVLHQDWLNGKEYKCKVS NKALPAPIEKTISKAKGQPREPQVYTLPPSRDELTKNQVSLT CLVKGFYPSDIAVEWESNGQPENNYKTTPPVLDSDGSFLLY SKLTVDKSRWQQGNVFSCSVMHEALHNHYTQKSLSLSPG K (SEQ ID NO:7) | Same as SEQ NO 6, but with linker and fc only |

TABLE 2

Constant regions for IgG3-like constructs

| | | |
|---|---|---|
| Construct | Sequence | Notes |
| WT huIgG3 CH1-CH2-CH3 | ASTKGPSVFPLAPCSRSTSGGTAALGCLVKDYFPEPVTVSW NSGALTSGVHTFPAVLQSSGLYSLSSVVTVPSSSLGTQTYTC NVNHKPSNTKVDKRVELKTPLGDTTHTPEPKSCDTPPPCPR CPAPELLGGPSVFLFPPKPKDTLMISRTPEVTCVVVDVSHE DPEVQFKWYVDGVEVHNAKTKPREEQYNSTFRVVSVLTVL HQDWLNGKEYKCKVSNKALPAPIEKTISKTKGQPREPQVY TLPPSREEMTKNQVSLTCLVKGFYPSDIAVEWESSGQPEN NYNTTPPMLDSDGSFFLYSKLTVDKSRWQQGNIFSCSVM HEALHNRFTQKSLSLSPGK (SEQ ID NO:8) | Hinge region underlined |
| V-IGG3 | ASTKGPSVFPLAPCSRSTSGGTAALGCLVKDYFPEPVTVSW NSGALTSGVHTFPAVLQSSGLYSLSSVVTVPSSSLGTQTYTC NVNHKPSNTKVDKRVESKYGPPCPPCPAPELLGGPSVFLFP PKPKDTLMISRTPEVTCVVVDVSHEDPEVQFKWYVDGVEV HNAKTKPREEQYNSTFRVVSVLTVLHQDWLNGKEYKCKVS NKALPAPIEKTISKTKGQPREPQVYTLPPSREEMTKNQVSLT CLVKGFYPSDIAVEWESSGQPENNYNTTPPMLDSDGSFFL YSKLTVDKSRWQQGNIFSCSVMHEALHNRFTQKSLSLSPG K (SEQ ID NO:9) | Hinge region underlined with IgG4 hinge S228P mutation in bold |
| V-IGG3-Fc | GGGGSGGGGSGGGGSKYGPPCPPCPAPELLGGPSVFLFPP KPKDTLMISRTPEVTCVVVDVSHEDPEVQFKWYVDGVEV HNAKTKPREEQYNSTFRVVSVLTVLHQDWLNGKEYKCKVS NKALPAPIEKTISKTKGQPREPQVYTLPPSREEMTKNQVSLT CLVKGFYPSDIAVEWESSGQPENNYNTTPPMLDSDGSFFL YSKLTVDKSRWQQGNIFSCSVMHEALHNRFTQKSLSLSPG K (SEQ ID NO:10) | Same as SEQ NO 9, but with linker and fc only |
| V-IGG3-A | ASTKGPSVFPLAPCSRSTSGGTAALGCLVKDYFPEPVTVSW NSGALTSGVHTFPAVLQSSGLYSLSSVVTVPSSSLGTQTYTC NVNHKPSNTKVDKRVESKYGPPCPPCPAPELLGGPSVFLFP PKPKDTLMISRTPEVTCVVVDVSHEDPEVQFKWYVDGVEV HNAKTKPREEQYNSTFRVVSVLTVLHQDWLNGKEYKCKVS NKALPAPIEKTISKTKGQPREPQVYTLPPSREEMTKNQVSLT CLVKGFYPSDIAVEWESSGQPENNYNTTPPMLDSDGSFFL YSRLTVDKSRWQQGNIFSCSVMHEALHNRFTQKSLSLSPG K (SEQ ID NO:11) | K409R |
| V-IGG3-A-Fc | GGGGSGGGGSGGGGSKYGPPCPPCPAPELLGGPSVFLFPP KPKDTLMISRTPEVTCVVVDVSHEDPEVQFKWYVDGVEV HNAKTKPREEQYNSTFRVVSVLTVLHQDWLNGKEYKCKVS NKALPAPIEKTISKTKGQPREPQVYTLPPSREEMTKNQVSLT CLVKGFYPSDIAVEWESSGQPENNYNTTPPMLDSDGSFFL YSRLTVDKSRWQQGNIFSCSVMHEALHNRFTQKSLSLSPG K (SEQ ID NO:12) | Same as SEQ NO 11, but with linker and fc only |
| V-IGG3-B | ASTKGPSVFPLAPCSRSTSGGTAALGCLVKDYFPEPVTVSW NSGALTSGVHTFPAVLQSSGLYSLSSVVTVPSSSLGTQTYTC NVNHKPSNTKVDKRVESKYGPPCPPCPAPELLGGPSVFLFP PKPKDTLMISRTPEVTCVVVDVSHEDPEVQFKWYVDGVEV HNAKTKPREEQYNSTFRVVSVLTVLHQDWLNGKEYKCKVS NKALPAPIEKTISKTKGQPREPQVYTLPPSREEMTKNQVSLT CLVKGFYPSDIAVEWESSGQPENNYNTTPPMLDSDGSFLL YSKLTVDKSRWQQGNIFSCSVMHEALHNRFTQKSLSLSPG K (SEQ ID NO:13) | F405L |
| V-IGG3-B-Fc | GGGGSGGGGSGGGGSKYGPPCPPCPAPELLGGPSVFLFPP KPKDTLMISRTPEVTCVVVDVSHEDPEVQFKWYVDGVEV HNAKTKPREEQYNSTFRVVSVLTVLHQDWLNGKEYKCKVS NKALPAPIEKTISKTKGQPREPQVYTLPPSREEMTKNQVSLT CLVKGFYPSDIAVEWESSGQPENNYNTTPPMLDSDGSFLL YSKLTVDKSRWQQGNIFSCSVMHEALHNRFTQKSLSLSPG K (SEQ ID NO:14) | Same as SEQ NO 13, but with linker and fc only |
| V-IGG3-C | ASTKGPSVFPLAPCSRSTSGGTAALGCLVKDYFPEPVTVSW NSGALTSGVHTFPAVLQSSGLYSLSSVVTVPSSSLGTQTYTC NVNHKPSNTKVDKRVELKTGDTTHTCPRCPAPELLGGPSV | Utilizes an IgG1-like hinge |
| | FLFPPKPKDTLMISRTPEVTCVVVDVSHEDPEVQFKWYVD GVEVHNAKTKPREEQYNSTFRVVSVLTVLHQDWLNGKEY KCKVSNKALPAPIEKTISKTKGQPREPQVYTLPPSREEMTKN QVSLTCLVKGFYPSDIAVEWESSGQPENNYNTTPPMLDSD GSFFLYSKLTVDKSRWQQGNIFSCSVMHEALHNRFTQKSL SLSPGK (SEQ ID NO:15) | |
| V-IGG3-C-Fc | GGGGSGGGGSGGGGSKTGDTTHTCPRCPAPELLGGPSVF LFPPKPKDTLMISRTPEVTCVVVDVSHEDPEVQFKWYVDG VEVHNAKTKPREEQYNSTFRVVSVLTVLHQDWLNGKEYKC KVSNKALPAPIEKTISKTKGQPREPQVYTLPPSREEMTKNQ VSLTCLVKGFYPSDIAVEWESSGQPENNYNTTPPMLDSDG SFFLYSKLTVDKSRWQQGNIFSCSVMHEALHNRFTQKSLSL SPGK (SEQ ID NO:16) | Same as SEQ NO 15, but with linker and fc only |
| V-IGG3-D | ASTKGPSVFPLAPCSRSTSGGTAALGCLVKDYFPEPVTVSW NSGALTSGVHTFPAVLQSSGLYSLSSVVTVPSSSLGTQTYTC NVNHKPSNTKVDKRVELKTGDTTHTCPRCPAPELLGGPSV FLFPPKPKDTLMISRTPEVTCVVVDVSHEDPEVQFKWYVD GVEVHNAKTKPREEQYNSTFRVVSVLTVLHQDWLNGKEY KCKVSNKALPAPIEKTISKTKGQPREPQVYTLPPSREEMTKN QVSLTCLVKGFYPSDIAVEWESSGQPENNYNTTPPMLDSD GSFFLYSRLTVDKSRWQQGNIFSCSVMHEALHNRFTQKSL SLSPGK (SEQ ID NO:17) | K409R |
| V-IGG3-D-Fc | GGGGSGGGGSGGGGSKTGDTTHTCPRCPAPELLGGPSVF LFPPKPKDTLMISRTPEVTCVVVDVSHEDPEVQFKWYVDG VEVHNAKTKPREEQYNSTFRVVSVLTVLHQDWLNGKEYKC KVSNKALPAPIEKTISKTKGQPREPQVYTLPPSREEMTKNQ VSLTCLVKGFYPSDIAVEWESSGQPENNYNTTPPMLDSDG SFFLYSRLTVDKSRWQQGNIFSCSVMHEALHNRFTQKSLSL SPGK (SEQ ID NO:18) | Same as SEQ NO 17, but with linker and fc only |
| V-IGG3-E | ASTKGPSVFPLAPCSRSTSGGTAALGCLVKDYFPEPVTVSW NSGALTSGVHTFPAVLQSSGLYSLSSVVTVPSSSLGTQTYTC NVNHKPSNTKVDKRVELKTGDTTHTCPRCPAPELLGGPSV FLFPPKPKDTLMISRTPEVTCVVVDVSHEDPEVQFKWYVD GVEVHNAKTKPREEQYNSTFRVVSVLTVLHQDWLNGKEY KCKVSNKALPAPIEKTISKTKGQPREPQVYTLPPSREEMTKN QVSLTCLVKGFYPSDIAVEWESSGQPENNYNTTPPMLDSD GSFLLYSKLTVDKSRWQQGNIFSCSVMHEALHNRFTQKSL SLSPGK (SEQ ID NO:19) | F405L |
| V-IGG3-E-Fc | GGGGSGGGGSGGGGSKTGDTTHTCPRCPAPELLGGPSVF LFPPKPKDTLMISRTPEVTCVVVDVSHEDPEVQFKWYVDG VEVHNAKTKPREEQYNSTFRVVSVLTVLHQDWLNGKEYKC KVSNKALPAPIEKTISKTKGQPREPQVYTLPPSREEMTKNQ | Same as SEQ NO 19, but with linker and fc only |
| | VSLTCLVKGFYPSDIAVEWESSGQPENNYNTTPPMLDSDG SFLLYSKLTVDKSRWQQGNIFSCSVMHEALHNRFTQKSLSL SPGK (SEQ ID NO:20) | |

TABLE 3

Sequences of anti-BBGL1 and anti-BBGL-2 constructs with enhanced ADCC, ADCP and CDC. CLK = kappa CL domain, CLL = lambda CL domain

| | | | | |
|---|---|---|---|---|
| Construct | Sequence of Chain 1 | Sequence of Chain 2 | Sequence of Chain 3 | Sequence of Chain 4 |
| VIT-203 | (BBGL1-VLL or BBGL1-VLK) - (CLK or CLL) | Same as Chain 1 | (BBGL1-VH) - (V-IGG1 or V-IGG3 or V-IGG3-C) | Same as Chain 3 |
| VIT-204 | (BBGL2-VLL or BBGL2-VLK) - (CLK or CLL) | Same as Chain 1 | (BBGL2-VH) - (V-IGG1 or V-IGG3 or V-IGG3-C) | Same as Chain 3 |
| VIT-205 | (BBGL1-VLL or BBGL1-VLK) - (CLK or CLL) | (BBGL2-VLL or BBGL2-VLK) - (CLK or CLL) | (BBGL1-VH) - (V-IGG1-A or V-IGG3-A or V-IGG3-D) | (BBGL2-VH)-(V-IGG1-B or V-IGG3-B or V-IGG3-E) |
| VIT-203sc | (BBGL1-VH-VLK-scFv or BBGL1-VH-VLL-scFv) - (V-IGG1-Fc or V-IGG3-Fc or V-IGG3-C-Fc) | Same as Chain 1 | | |
| VIT-204sc | (BBGL2-VH-VLK-scFv or BBGL2-VH-VLL-scFv)- (V-IGG1-Fc or V-IGG3-Fc or V-IGG3-C-Fc) | Same as Chain 1 | | |
| VIT-205sc | (BBGL1-VH-VLK-scFv or BBGL1-VH-VLL-scFv) - (V-IGG1-A-Fc or V-IGG3-A-Fc or V-IGG3-D-Fc) | (BBGL2-VH-VLK-scFv or BBGL2-VH-VLL-scFv)- (V-IGG1-B-Fc or V-IGG3-B-Fc or V-IGG3-E-Fc) | | |

### Example 3: Design of Anti-BBGL-1 and Anti-BBGL-2 Antibodies with T Cell Engager Effector Functions

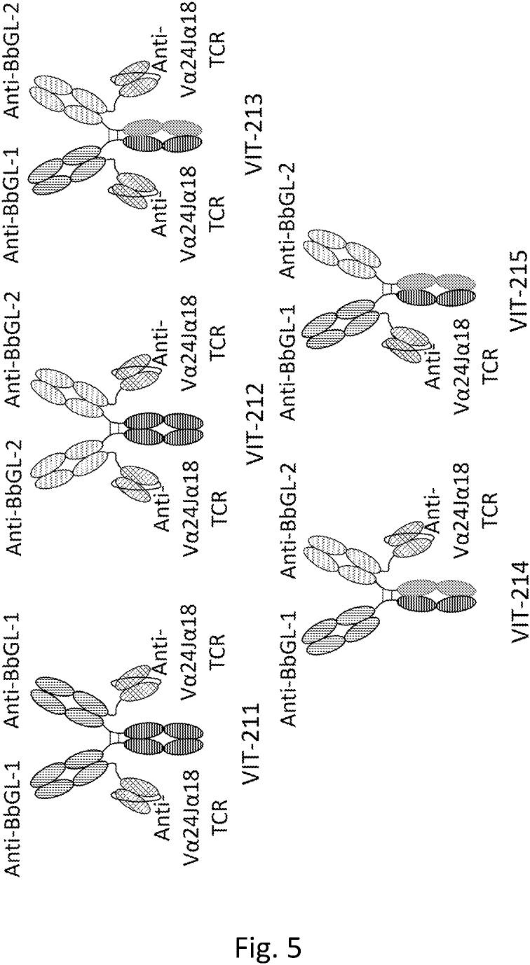

T cell engaging bispecific antibodies were designed by fusing a GlySer linker (GGGGSGGGGSGGGGS (=3xSEQ ID NO: 92)) and anti-CD3 scFv to C-terminus of the light chain of the anti-BBGL-1/BBGL-2 Fab domain (see FIG. 4). Null Fc fragments are utilized using IgG4 or IgG2 Fc region.

TABLE 4

Sequences of anti-BBGL1 and anti-BBGL-2 constructs with T cell effector function. CLK = kappa CL domain, CLL = lambda CL domain

| | | | | |
|---|---|---|---|---|
| Construct | Sequence of Chain 1 | Sequence of Chain 2 | Sequence of Chain 3 | Sequence of Chain 4 |
| VIT-206 | (BBGL1-VLL or BBGL1-VLK) - (CLK or CLL)-GGGGSGGGGSGG GGSGGGGS(=4X SEQ ID NO: 92)-(any scFv from Table 5) | Same as Chain 1 | (BBGL1-VH)-(V-IGG4-A or V-IGG2 or V-IGG2-C) | Same as Chain 3 |
| VIT-207 | (BBGL2-VLL or BBGL2-VLK) - (CLK or CLL)-GGGGSGGGGSGG GGSGGGGS(=4X SEQ ID NO: 92)-(any scFv from Table 5) | Same as Chain 1 | (BBGL2-VH)-(V-IGG4-A or V-IGG2 or V-IGG2-C) | Same as Chain 3 |
| VIT-208 | (BBGL1-VLL or BBGL1-VLK) - (CLK or CLL)-GGGGSGGGGSGG GGSGGGGS(=4X SEQ ID NO: 92)-(any scFv from Table 5) | (BBGL2-VLL or BBGL2-VLK) - (CLK or CLL)-GGGGSGGGGSGG GGSGGGGS(=4X SEQ ID NO: 92)-(any scFv from Table 5) | (BBGL1-VH)-(V-IGG4-A or V-IGG2-A or V-IGG2-D) | (BBGL2-VH)-(V-IGG4-B or V-IGG2-B or V-IGG2-E) |
| VIT-209 | (BBGL1-VLL or BBGL1-VLK) - (CLK or CLL) | (BBGL2-VLL or BBGL2-VLK) - (CLK or CLL)-GGGGSGGGGSGG GGSGGGGS(=4X | (BBGL1-VH)- | (BBGL2-VH)- |
| | | SEQ ID NO: 92)-(any scFv from Table 5) | (V-IGG4-A or V-IGG2-A or V-IGG2-D) | (V-IGG4-B or V-IGG2-B or V-IGG2-E) |
| VIT210 | (BBGL1-VLL or BBGL1-VLK) - (CLK or CLL)-GGGGSGGGGSGG GGSGGGGS(=4X SEQ ID NO: 92)-(any scFv from Table 5) | (BBGL2-VLL or BBGL2-VLK) - (CLK or CLL) | (BBGL1-VH)-(V-IGG4-A or V-IGG2-A or V-IGG2-D) | (BBGL2-VH)-(V-IGG4-B or V-IGG2-B or V-IGG2-E) |

TABLE 5

Anti-CD3 scFv sequences

| | |
|---|---|
| Construct | Sequence |
| VCD3-H1L1 | QVQLVQSGGGVVQPGRSLRLSCAASGYTFTRYTMHWVRQAPGKGLEWVGYINPSRGYTNYND SVKGRFTISTDKSKNTAYLQMNSLRAEDTAVYYCARYYDDHYSLDYWGQGTTVTVSSGGGGSG GGGSGGGGSGGGGSGGGGSGGGGS DIQMTQSPSSLSASVGDRVTITCQASSSVSYMNWYQQKPGKAPKRWIYDTSKLASGVPSRFSGS GSGTDYTFTISSLQPEDIATYYCQQWSSNPFTFGQGTKLEIK (SEQ ID NO:21) |
| VCD3-H1L1-DS | QVQLVQSGGGVVQPGRSLRLSCAASGYTFTRYTMHWVRQAPGKCLEWVGYINPSRGYTNYND SVKGRFTISTDKSKNTAYLQMNSLRAEDTAVYYCARYYDDHYSLDYWGQGTTVTVSSGGGGSG GGGSGGGGSGGGGSGGGGSGGGGS DIQMTQSPSSLSASVGDRVTITCQASSSVSYMNWYQQKPGKAPKRWIYDTSKLASGVPSRFSGS GSGTDYTFTISSLQPEDIATYYCQQWSSNPFTFGCGTKLEIK (SEQ ID NO:22) |
| VCD3-H1L2 | QVQLVQSGGGVVQPGRSLRLSCAASGYTFTRYTMHWVRQAPGKGLEWVGYINPSRGYTNYND SVKGRFTISTDKSKNTAYLQMNSLRAEDTAVYYCARYYDDHYSLDYWGQGTTVTVSSGGGGSG GGGSGGGGSGGGGSGGGGSGGGGS EIVLTQSPATLSLSPGERATLSCRASSSVSYMNWYQQKPGQAPRRLIYDTSKRATGIPARFSGSGS GTDYTLTISSLEPEDAAVYYCQQWSSNPFTFGQGTKLEIK (SEQ ID NO:23) |
| VCD3-H1L2-DS | QVQLVQSGGGVVQPGRSLRLSCAASGYTFTRYTMHWVRQAPGKCLEWVGYINPSRGYTNYND SVKGRFTISTDKSKNTAYLQMNSLRAEDTAVYYCARYYDDHYSLDYWGQGTTVTVSSGGGGSG GGGSGGGGSGGGGSGGGGSGGGGS EIVLTQSPATLSLSPGERATLSCRASSSVSYMNWYQQKPGQAPRRLIYDTSKRATGIPARFSGSGS GTDYTLTISSLEPEDAAVYYCQQWSSNPFTFGCGTKLEIK (SEQ ID NO:24) |
| VCD3-H1L3 | QVQLVQSGGGVVQPGRSLRLSCAASGYTFTRYTMHWVRQAPGKGLEWVGYINPSRGYTNYND SVKGRFTISTDKSKNTAYLQMNSLRAEDTAVYYCARYYDDHYSLDYWGQGTTVTVSSGGGGSG GGGSGGGGSGGGGSGGGGSGGGGS |
| | EIQLTQSPATLSLSPGERATLSCRASSSVSYMNWYQQKPGQAPRRWIYDTSKLATGIPARFSGSG SGTDYTLTISSLEPEDAAVYYCQQWSSNPFTFGQGTKLEIK (SEQ ID NO:25) |
| VCD3-H1L3-DS | QVQLVQSGGGVVQPGRSLRLSCAASGYTFTRYTMHWVRQAPGKCLEWVGYINPSRGYTNYND SVKGRFTISTDKSKNTAYLQMNSLRAEDTAVYYCARYYDDHYSLDYWGQGTTVTVSSGGGGSG GGGSGGGGSGGGGSGGGGSGGGGS EIQLTQSPATLSLSPGERATLSCRASSSVSYMNWYQQKPGQAPRRWIYDTSKLATGIPARFSGSG SGTDYTLTISSLEPEDAAVYYCQQWSSNPFTFGCGTKLEIK (SEQ ID NO:26) |
| VCD3-H2L1 | QVQLVQSGAEVKKPGASVKVSCKASGYTFTRYTMHWVRQAPGQGLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS DIQMTQSPSSLSASVGDRVTITCQASSSVSYMNWYQQKPGKAPKRWIYDTSKLASGVPSRFSGS GSGTDYTFTISSLQPEDIATYYCQQWSSNPFTFGQGTKLEIK (SEQ ID NO:27) |
| VCD3-H2L1-DS | QVQLVQSGAEVKKPGASVKVSCKASGYTFTRYTMHWVRQAPGQCLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS DIQMTQSPSSLSASVGDRVTITCQASSSVSYMNWYQQKPGKAPKRWIYDTSKLASGVPSRFSGS GSGTDYTFTISSLQPEDIATYYCQQWSSNPFTFGCGTKLEIK (SEQ ID NO:28) |
| VCD3-H2L2 | QVQLVQSGAEVKKPGASVKVSCKASGYTFTRYTMHWVRQAPGQGLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS EIVLTQSPATLSLSPGERATLSCRASSSVSYMNWYQQKPGQAPRRLIYDTSKRATGIPARFSGSGS GTDYTLTISSLEPEDAAVYYCQQWSSNPFTFGQGTKLEIK (SEQ ID NO:29) |
| VCD3-H2L2-DS | QVQLVQSGAEVKKPGASVKVSCKASGYTFTRYTMHWVRQAPGQCLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS EIVLTQSPATLSLSPGERATLSCRASSSVSYMNWYQQKPGQAPRRLIYDTSKRATGIPARFSGSGS GTDYTLTISSLEPEDAAVYYCQQWSSNPFTFGCGTKLEIK (SEQ ID NO:30) |
| VCD3-H2L3 | QVQLVQSGAEVKKPGASVKVSCKASGYTFTRYTMHWVRQAPGQGLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS EIQLTQSPATLSLSPGERATLSCRASSSVSYMNWYQQKPGQAPRRWIYDTSKLATGIPARFSGSG SGTDYTLTISSLEPEDAAVYYCQQWSSNPFTFGQGTKLEIK (SEQ ID NO:31) |
| VCD3-H2L3-DS | QVQLVQSGAEVKKPGASVKVSCKASGYTFTRYTMHWVRQAPGQCLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS EIQLTQSPATLSLSPGERATLSCRASSSVSYMNWYQQKPGQAPRRWIYDTSKLATGIPARFSGSG SGTDYTLTISSLEPEDAAVYYCQQWSSNPFTFGCGTKLEIK (SEQ ID NO:32) |
| VCD3-H3L1 | QVQLVQSGAEVKKPGASVKVSCKASGYTFTRYTMHWVRQSPGQGLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS |
| | DIQMTQSPSSLSASVGDRVTITCQASSSVSYMNWYQQKPGKAPKRWIYDTSKLASGVPSRFSGS GSGTDYTFTISSLQPEDIATYYCQQWSSNPFTFGQGTKLEIK (SEQ ID NO:33) |
| VCD3-H3L1-DS | QVQLVQSGAEVKKPGASVKVSCKASGYTFTRYTMHWVRQSPGQCLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS DIQMTQSPSSLSASVGDRVTITCQASSSVSYMNWYQQKPGKAPKRWIYDTSKLASGVPSRFSGS GSGTDYTFTISSLQPEDIATYYCQQWSSNPFTFGCGTKLEIK (SEQ ID NO:34) |
| VCD3-H3L2 | QVQLVQSGAEVKKPGASVKVSCKASGYTFTRYTMHWVRQSPGQGLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS EIVLTQSPATLSLSPGERATLSCRASSSVSYMNWYQQKPGQAPRRLIYDTSKRATGIPARFSGSGS GTDYTLTISSLEPEDAAVYYCQQWSSNPFTFGQGTKLEIK (SEQ ID NO:35) |
| VCD3-H3L2-DS | QVQLVQSGAEVKKPGASVKVSCKASGYTFTRYTMHWVRQSPGQCLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS EIVLTQSPATLSLSPGERATLSCRASSSVSYMNWYQQKPGQAPRRLIYDTSKRATGIPARFSGSGS GTDYTLTISSLEPEDAAVYYCQQWSSNPFTFGCGTKLEIK (SEQ ID NO:36) |
| VCD3-H3L3 | QVQLVQSGAEVKKPGASVKVSCKASGYTFTRYTMHWVRQSPGQGLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS EIQLTQSPATLSLSPGERATLSCRASSSVSYMNWYQQKPGQAPRRWIYDTSKLATGIPARFSGSG SGTDYTLTISSLEPEDAAVYYCQQWSSNPFTFGQGTKLEIK (SEQ ID NO:37) |
| VCD3-H3L3-DS | QVQLVQSGAEVKKPGASVKVSCKASGYTFTRYTMHWVRQSPGQCLEWMGYINPSRGYTNYN QKFQGRVTMTTDKSTSTAYMELSSLRSEDTAVYYCARYYDDHYSLDYWGQGTTVTVSSGGGGS GGGGSGGGGSGGGGSGGGGSGGGGS EIQLTQSPATLSLSPGERATLSCRASSSVSYMNWYQQKPGQAPRRWIYDTSKLATGIPARFSGSG SGTDYTLTISSLEPEDAAVYYCQQWSSNPFTFGCGTKLEIK (SEQ ID NO:38) |

TABLE 6

Sequences for null Fc domains and heterodimerization

| | | |
|---|---|---|
| Construct | Sequence | Notes |
| WT huIgG1 CH1-CH2-CH3 | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKKVEPKSCDKTHTCPPCPAPELLGGPSVFLFPPKPKDTL MISRTPEVTCVVVDVSHEDPEVKFNWYVDGVEVHNAKTKPRE EQYNSTYRVVSVLTVLHQDWLNGKEYKCKVSNKALPAPIEKTIS KAKGQPREPQVYTLPPSRDELTKNQVSLTCLVKGFYPSDIAVEW ESNGQPENNYKTTPPVLDSDGSFFLYSKLTVDKSRWQQGNVFS CSVMHEALHNHYTQKSLSLSPGK (SEQ ID NO:39) | |
| WT huIgG4 CH1-CH2-CH3 | ASTKGPSVFPLAPCSRSTSESTAALGCLVKDYFPEPVTVSWNSG ALTSGVHTFPAVLQSSGLYSLSSVVTVPSSSLGTKTYTCNVDHKP SNTKVDKRVESKYGPPCPSCPAPEFLGGPSVFLFPPKPKDTLMIS RTPEVTCVVVDVSQEDPEVQFNWYVDGVEVHNAKTKPREEQF NSTYRVVSVLTVLHQDWLNGKEYKCKVSNKGLPSSIEKTISKAK GQPREPQVYTLPPSQEEMTKNQVSLTCLVKGFYPSDIAVEWES NGQPENNYKTTPPVLDSDGSFFLYSRLTVDKSRWQEGNVFSCS VMHEALHNHYTQKSLSLSLGK (SEQ ID NO:40) | |
| V-IGG4-A | ASTKGPSVFPLAPCSRSTSESTAALGCLVKDYFPEPVTVSWNSG ALTSGVHTFPAVLQSSGLYSLSSVVTVPSSSLGTKTYTCNVDHKP SNTKVDKRVESKYGPPCPPCPAPEAAGGPSVFLFPPKPKDTLMI SRTPEVTCVVVDVSQEDPEVQFNWYVDGVEVHNAKTKPREEQ FNSTYRVVSVLTVLHQDWLNGKEYKCAVSNKGLPSSIEKTISKAK GQPREPQVYTLPPSQEEMTKNQVSLTCLVKGFYPSDIAVEWES NGQPENNYKTTPPVLDSDGSFFLYSRLTVDKSRWQEGNVFSCS VMHEALHNHYTQKSLSLSLGK (SEQ ID NO:41) | S228P FALA (F234A, L235A) K322A Naturally contains F405 and R409 |
| V-IGG4-A-Fc | GGGGSGGGGSGGGGSKYGPPCPPCPAPEAAGGPSVFLFPPKP KDTLMISRTPEVTCVVVDVSQEDPEVQFNWYVDGVEVHNAKT KPREEQFNSTYRVVSVLTVLHQDWLNGKEYKCAVSNKGLPSSIE KTISKAKGQPREPQVYTLPPSQEEMTKNQVSLTCLVKGFYPSDI AVEWESNGQPENNYKTTPPVLDSDGSFFLYSRLTVDKSRWQE GNVFSCSVMHEALHNHYTQKSLSLSLGK (SEQ ID NO:42) | Same as above, but with linker and fc only |
| V-IGG4-B | ASTKGPSVFPLAPCSRSTSESTAALGCLVKDYFPEPVTVSWNSG ALTSGVHTFPAVLQSSGLYSLSSVVTVPSSSLGTKTYTCNVDHKP SNTKVDKRVESKYGPPCPPCPAPEAAGGPSVFLFPPKPKDTLMI SRTPEVTCVVVDVSQEDPEVQFNWYVDGVEVHNAKTKPREEQ FNSTYRVVSVLTVLHQDWLNGKEYKCAVSNKGLPSSIEKTISKAK GQPREPQVYTLPPSQEEMTKNQVSLTCLVKGFYPSDIAVEWES NGQPENNYKTTPPVLDSDGSFLLYSKLTVDKSRWQEGNVFSCS VMHEALHNHYTQKSLSLSLGK (SEQ ID NO:43) | S228P FALA (F234A, L235A) K322A F405L, R409K |
| V-IGG4-B-Fc | GGGGSGGGGSGGGGSKYGPPCPPCPAPEAAGGPSVFLFPPKP KDTLMISRTPEVTCVVVDVSQEDPEVQFNWYVDGVEVHNAKT KPREEQFNSTYRVVSVLTVLHQDWLNGKEYKCAVSNKGLPSSIE KTISKAKGQPREPQVYTLPPSQEEMTKNQVSLTCLVKGFYPSDI AVEWESNGQPENNYKTTPPVLDSDGSFLLYSKLTVDKSRWQE GNVFSCSVMHEALHNHYTQKSLSLSLGK (SEQ ID NO:44) | Same as above, but with linker and fc only |
| WT huIgG2 CH1-CH2-CH3 | ASTKGPSVFPLAPCSRSTSESTAALGCLVKDYFPEPVTVSWNSG ALTSGVHTFPAVLQSSGLYSLSSVVTVPSSNFGTQTYTCNVDHK PSNTKVDKTVERKCCVECPPCPAPPVAGPSVFLFPPKPKDTLMI SRTPEVTCVVVDVSHEDPEVQFNWYVDGVEVHNAKTKPREEQ FNSTFRVVSVLTVVHQDWLNGKEYKCKVSNKGLPAPIEKTISKT KGQPREPQVYTLPPSREEMTKNQVSLTCLVKGFYPSDISVEWES | |
| | NGQPENNYKTTPPMLDSDGSFFLYSKLTVDKSRWQQGNVFSC SVMHEALHNHYTQKSLSLSPGK (SEQ ID NO:45) | |
| V-IGG2 | ASTKGPSVFPLAPCSRSTSESTAALGCLVKDYFPEPVTVSWNSG ALTSGVHTFPAVLQSSGLYSLSSVVTVPSSNFGTQTYTCNVDHK PSNTKVDKTVERKDKTHTCPPCPAPPAAGPSVFLFPPKPKDTLM ISRTPEVTCVVVDVSHEDPEVQFNWYVDGVEVHNAKTKPREE QFNSTFRVVSVLTVVHQDWLNGKEYKCAVSNKGLPAPIEKTISK TKGQPREPQVYTLPPSREEMTKNQVSLTCLVKGFYPSDISVEWE SNGQPENNYKTTPPMLDSDGSFFLYSKLTVDKSRWQQGNVFS CSVMHEALHNHYTQKSLSLSPGK (SEQ ID NO:46) | Contains IgG1 hinge (DKTHTCPPCPAP) VA (V234A) K322A |
| V-IGG2-Fc | GGGGSGGGGSGGGGSDKTHTCPPCPAPPAAGPSVFLFPPKPK DTLMISRTPEVTCVVVDVSHEDPEVQFNWYVDGVEVHNAKTK PREEQFNSTFRVVSVLTVVHQDWLNGKEYKCAVSNKGLPAPIE KTISKTKGQPREPQVYTLPPSREEMTKNQVSLTCLVKGFYPSDIS VEWESNGQPENNYKTTPPMLDSDGSFFLYSKLTVDKSRWQQG NVFSCSVMHEALHNHYTQKSLSLSPGK (SEQ ID NO:47) | Same as above, but with linker and fc only |
| V-IGG2-A | ASTKGPSVFPLAPCSRSTSESTAALGCLVKDYFPEPVTVSWNSG ALTSGVHTFPAVLQSSGLYSLSSVVTVPSSNFGTQTYTCNVDHK PSNTKVDKTVERKDKTHTCPPCPAPPAAGPSVFLFPPKPKDTLM ISRTPEVTCVVVDVSHEDPEVQFNWYVDGVEVHNAKTKPREE QFNSTFRVVSVLTVVHQDWLNGKEYKCAVSNKGLPAPIEKTISK TKGQPREPQVYTLPPSREEMTKNQVSLTCLVKGFYPSDISVEWE SNGQPENNYKTTPPMLDSDGSFFLYSRLTVDKSRWQQGNVFS CSVMHEALHNHYTQKSLSLSPGK (SEQ ID NO:48) | Contains IgG1 hinge (DKTHTCPPCPAP) VA (V234A) K322A K409R |
| V-IGG2-A-Fc | GGGGSGGGGSGGGGSDKTHTCPPCPAPPAAGPSVFLFPPKPK DTLMISRTPEVTCVVVDVSHEDPEVQFNWYVDGVEVHNAKTK PREEQFNSTFRVVSVLTVVHQDWLNGKEYKCAVSNKGLPAPIE KTISKTKGQPREPQVYTLPPSREEMTKNQVSLTCLVKGFYPSDIS VEWESNGQPENNYKTTPPMLDSDGSFFLYSRLTVDKSRWQQG NVFSCSVMHEALHNHYTQKSLSLSPGK (SEQ ID NO:49) | Same as above, but with linker and fc only |
| V-IGG2-B | ASTKGPSVFPLAPCSRSTSESTAALGCLVKDYFPEPVTVSWNSG ALTSGVHTFPAVLQSSGLYSLSSVVTVPSSNFGTQTYTCNVDHK PSNTKVDKTVERKDKTHTCPPCPAPPAAGPSVFLFPPKPKDTLM ISRTPEVTCVVVDVSHEDPEVQFNWYVDGVEVHNAKTKPREE QFNSTFRVVSVLTVVHQDWLNGKEYKCAVSNKGLPAPIEKTISK TKGQPREPQVYTLPPSREEMTKNQVSLTCLVKGFYPSDISVEWE SNGQPENNYKTTPPMLDSDGSFLLYSKLTVDKSRWQQGNVFS CSVMHEALHNHYTQKSLSLSPGK (SEQ ID NO:50) | Contains IgG1 hinge (DKTHTCPPCPAP) FALA (F234A) K322A F405L |
| V-IGG2-B-Fc | GGGGSGGGGSGGGGSDKTHTCPPCPAPPAAGPSVFLFPPKPK DTLMISRTPEVTCVVVDVSHEDPEVQFNWYVDGVEVHNAKTK PREEQFNSTFRVVSVLTVVHQDWLNGKEYKCAVSNKGLPAPIE KTISKTKGQPREPQVYTLPPSREEMTKNQVSLTCLVKGFYPSDIS VEWESNGQPENNYKTTPPMLDSDGSFLLYSKLTVDKSRWQQG NVFSCSVMHEALHNHYTQKSLSLSPGK (SEQ ID NO:51) | Same as above, but with linker and fc only |
| V-IGG2-C | ASTKGPSVFPLAPCSRSTSESTAALGCLVKDYFPEPVTVSWNSG ALTSGVHTFPAVLQSSGLYSLSSVVTVPSSNFGTQTYTCNVDHK PSNTKVDKTVERKYGPPCPPCPAPPAAGPSVFLFPPKPKDTLMI SRTPEVTCVVVDVSHEDPEVQFNWYVDGVEVHNAKTKPREEQ FNSTFRVVSVLTVVHQDWLNGKEYKCAVSNKGLPAPIEKTISKT KGQPREPQVYTLPPSREEMTKNQVSLTCLVKGFYPSDISVEWES NGQPENNYKTTPPMLDSDGSFFLYSKLTVDKSRWQQGNVFSC SVMHEALHNHYTQKSLSLSPGK (SEQ ID NO:52) | Contains IgG4 hinge (YGPPCPPCPAP) with S228P FALA (V234A) K322A |
| V-IGG2-C-Fc | GGGGSGGGGSGGGGSYGPPCPPCPAPPAAGPSVFLFPPKPKD TLMISRTPEVTCVVVDVSHEDPEVQFNWYVDGVEVHNAKTKP REEQFNSTFRVVSVLTVVHQDWLNGKEYKCAVSNKGLPAPIEK TISKTKGQPREPQVYTLPPSREEMTKNQVSLTCLVKGFYPSDISV EWESNGQPENNYKTTPPMLDSDGSFFLYSKLTVDKSRWQQG NVFSCSVMHEALHNHYTQKSLSLSPGK (SEQ ID NO:53) | Same as above, but with linker and fc only |
| V-IGG2-D | ASTKGPSVFPLAPCSRSTSESTAALGCLVKDYFPEPVTVSWNSG ALTSGVHTFPAVLQSSGLYSLSSVVTVPSSNFGTQTYTCNVDHK PSNTKVDKTVERKYGPPCPPCPAPPAAGPSVFLFPPKPKDTLMI SRTPEVTCVVVDVSHEDPEVQFNWYVDGVEVHNAKTKPREEQ FNSTFRVVSVLTVVHQDWLNGKEYKCAVSNKGLPAPIEKTISKT KGQPREPQVYTLPPSREEMTKNQVSLTCLVKGFYPSDISVEWES NGQPENNYKTTPPMLDSDGSFFLYSRLTVDKSRWQQGNVFSC SVMHEALHNHYTQKSLSLSPGK (SEQ ID NO:54) | Contains IgG4 hinge (YGPPCPPCPAP) with S228P VA (V234A) K322A K409R |
| V-IGG2-D-Fc | GGGGSGGGGSGGGGSYGPPCPPCPAPPAAGPSVFLFPPKPKD TLMISRTPEVTCVVVDVSHEDPEVQFNWYVDGVEVHNAKTKP REEQFNSTFRVVSVLTVVHQDWLNGKEYKCAVSNKGLPAPIEK TISKTKGQPREPQVYTLPPSREEMTKNQVSLTCLVKGFYPSDISV EWESNGQPENNYKTTPPMLDSDGSFFLYSRLTVDKSRWQQG NVFSCSVMHEALHNHYTQKSLSLSPGK (SEQ ID NO:55) | Same as above, but with linker and fc only |
| V-IGG2-E | ASTKGPSVFPLAPCSRSTSESTAALGCLVKDYFPEPVTVSWNSG ALTSGVHTFPAVLQSSGLYSLSSVVTVPSSNFGTQTYTCNVDHK PSNTKVDKTVERKYGPPCPPCPAPPAAGPSVFLFPPKPKDTLMI SRTPEVTCVVVDVSHEDPEVQFNWYVDGVEVHNAKTKPREEQ FNSTFRVVSVLTVVHQDWLNGKEYKCAVSNKGLPAPIEKTISKT KGQPREPQVYTLPPSREEMTKNQVSLTCLVKGFYPSDISVEWES NGQPENNYKTTPPMLDSDGSFLLYSKLTVDKSRWQQGNVFSC SVMHEALHNHYTQKSLSLSPGK (SEQ ID NO:56) | Contains IgG4 hinge (YGPPCPPCPAP) with S228P FALA (FV34A) K322A F405L |
| V-IGG2-E-Fc | GGGGSGGGGSGGGGSYGPPCPPCPAPPAAGPSVFLFPPKPKD TLMISRTPEVTCVVVDVSHEDPEVQFNWYVDGVEVHNAKTKP REEQFNSTFRVVSVLTVVHQDWLNGKEYKCAVSNKGLPAPIEK TISKTKGQPREPQVYTLPPSREEMTKNQVSLTCLVKGFYPSDISV EWESNGQPENNYKTTPPMLDSDGSFLLYSKLTVDKSRWQQGN VFSCSVMHEALHNHYTQKSLSLSPGK (SEQ ID NO:57) | Same as above, but with linker and fc only |

### Example 4: Design of Anti-BBGL-1 and Anti-BBGL-2 Antibodies with NKT Cell Engager Effector Functions

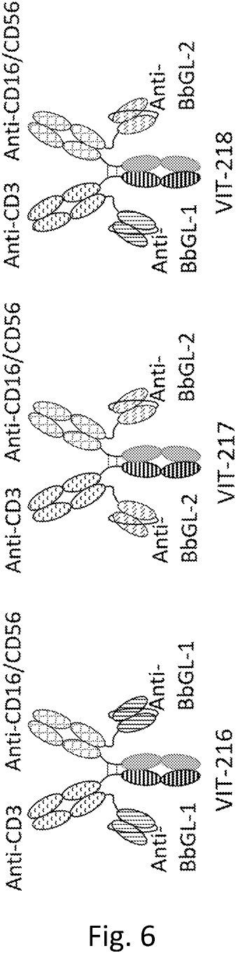

NKT cell engaging bispecific antibodies were designed by fusing a GlySer linker (GGGGSGGGGSGGGGS (=3x SEQ ID NO: 92)) and anti- TCR Vα24Jα18 scFv to C-terminus of the light chain of the anti-BBGL-1/BBGL-2 Fab domain (see FIG. 5). Vα24Jα18 encodes the TCR-alpha chain of invariant NKT cells, and humanized anti- TCR Vα24Jα18 mAb was utilized to generate scFv sequences. Null Fc fragments are incorporated using modified IgG4 or IgG2 fragments.

Sequence of Valpha24 might be as follows:

TVA24_HUMAN T cell receptor alpha variable (residues 23-114)

ILNVEQSPQSLHVQEGDSTNFTCSFPSSNFYALHWYRWETAKSPEALFVM
TLNGDEKKKGRISATLNTKEGYSYLYIKGSQPEDSATYLCAF (SEQ ID
NO:81).

Sequence of TRAJ18, T cell receptor alpha joining 18:

DRGSTLGRLYFGRGTQLTVWP (SEQ ID NO:82).

Together: TCR Vα24Jα18

ILNVEQSPQSLHVQEGDSTNFTCSFPSSNFYALHWYRWETAKSPEALFVM
TLNGDEKKKGRISATLNTKEGYSYLYIKGSQPEDSATYLCAFDRGSTLGR
LYFGRGTQLTVWP (SEQ ID NO:83).

Anti- human TCR Vα24Jα18 mAb 6B11 (https://www.ncbi.nlm.nih.gov/pmc/articles/PMC2864538/) was humanized. The 6B11 sequence is listed below, with IMGT CDRs underlined:

VL:

DIVMTQSHKFMSTSVGDRVSITCKASQDVSTAVAWYQQKPGQSPKLLIYW
ASTRHTGVPDRFTGSGSGTDYTLTISSVQAEDLALYYCQQHYSTPWTFGG
GTKLEIK (SEQ ID NO:58)

6B11 VL CDR1: QDVSTA (SEQ ID NO:84), 6B11 VL CDR2: WAS (SEQ ID NO:85), 6B11 VL CDR3:
QQHYSTPWT (SEQ ID NO:86).

6B11 VH CDR1: GFTFSNYW (SEQ ID NO:87), 6B11 VH CDR2: IRLKSNNYAT (SEQ ID NO:88),
6B11 VH CDR3: TRNGNYVDYAMDY (SEQ ID NO:89).

VH:

EVKLEESGGGLVQPGGSMKLSCVASGFTFSNYWMNWVRQSPEKGLEWVAE
IRLKSNNYATHYAESVKGRFTISRDDSKSSVYLQMNNLRAEDTGIYYCTR
NGNYVDYAMDYWGQGTSVTVSS (SEQ ID NO:61)

Potentially immunogenic peptides were aimed to be minimized in the humanized sequences.

Humanizing mutations were made based on identity to the closest human germline sequence and predicted ability to have high protein stability, retain high affinity binding to target antigen, while minimizing potential aggregation and minimizing potential immunogenicity. Final constructs are presented in Table 1 and Table 2.

A previously humanized 6B11, mAb NKTT120, was included for comparison.

TABLE 7

human TCR Vα24Jα18 VL sequences

| | | | | |
|---|---|---|---|---|
| Construct | Sequence | % human germline | Predicted immunogenic peptides | Notes |
| m6B11 VL | DIVMTQSHKFMST SVGDRVSITCKASQ DVSTAVAWYQQK PGQSPKLLIYWAST RHTGVPDRFTGSG SGTDYTLTISSVQAE | 69.5% IGKV1-39*01 | DIVMTQSHKFMSTS V (amino acids 1-15 of SEQ ID NO:; 58) | |
| | DLALYYCQQHYSTP WTFGGGTKLEIK (SEQ ID NO:58) | | LLIYWASTRHTGVPD (amino acids 46-60 of SEQ ID NO: 58) | |
| NKTT120 VL | DIQMTQSPSSLSAS VGDRVTITCKASQD VSTAVAWYQQKP GQAPRLLIYWASTR HTGVPSRFSGSGSG TDFTLTISSLQPEDF ALYYCQQHYSTPW TFGQGTKLEIK (SEQ ID NO:59) | 83.2% IGKV1-39*01 | LLIYWASTRHTGVPS (amino acids 46-60 of SEQ ID NO: 59) | |
| VTCR-L1 | DIQMTQSPSSLSAS VGDRVTITCRASQD VSTAVAWYQQKP GKAPKLLIYWASTR HTGVPSRFSGSGSG TDYTLTISSLQPEDF ATYYCQQHYSTPW TFGGGTKVEIK (SEQ ID NO:60) | 86.3% IGKV1-39*01 | LLIYWASTRHTGVPS (amino acids 46-60 of SEQ ID NO: 60) | |

TABLE 8

Anti- human TCR Va24Ja18 VH sequences

| | | | | |
|---|---|---|---|---|
| Construct | Sequence | % human germline | Predicted immunogenic peptides | Notes |
| m6B11 VH | EVKLEESGGGLVQ PGGSMKLSCVAS GFTFSNYWMNW VRQSPEKGLEWV AEIRLKSNNYATH YAESVKGRFTISRD DSKSSVYLQMNN LRAEDTGIYYCTRN GNYVDYAMDYW GQGTSVTVSS (SEQ ID NO:61) | 74% IGHV3-73*01 | EWVAEIRLKSNNYA T (amino acids 46-60 of SEQ ID NO: 61) DSKSSVYLQMNNLR A (amino acids 76-90 of SEQ ID NO: 61) VYLQMNNLRAEDT GI (amino acids 81-95 of SEQ ID NO: 61) | |
| NKTT120 VH | EVQLVESGGGLV QPGGSLRLSCVAS GFTFSNYWMNW VRQAPGKGLEWV AEIRLKSNNYATH YAESVKGRFTISRD DSKNTVYLQMNS LRAEDTAVYYCTR NGNYVDYAMDY WGQGTLVTVSS (SEQ ID NO:62) | 83% IGHV3-73*01 | EWVAEIRLKSNNYA T (amino acids 46-60 of SEQ ID NO: 62) DSKNTVYLQMNSLR A (amino acids 76-90 of SEQ ID NO: 62) VYLQMNSLRAEDTA V (amino acids 81-95 of SEQ ID NO: 62) | |
| VTCR-H1 | EVQLVESGGGLV QPGGSLKLSCAAS GFTFSNYWMNW VRQAPGKGLEWV AEIRLKSNNYATH YAESVKGRFTISRD DSKNTAYLQMNS LKTEDTAVYYCTR NGNYVDYAMDY WGQGTLVTVSS (SEQ ID NO:63) | 88% IGHV3-73*01 | EWVAEIRLKSNNYA T (amino acids 46-60 of SEQ ID NO: 63) DSKNTAYLQMNSLK T (amino acids 76-90 of SEQ ID NO: 63) | |

Single chain variable fragments (scFv) were designed based on a VH-VL orientation and are presented in Table 9. Additional disulfide stabilization between the VH and VL domains was engineered by substituting Cys at positions VH44-VL100 (Kabat numbering).

TABLE 9

Anti- human TCR Va24Ja18 scFv sequences

| | |
|---|---|
| Construct | Sequence |
| VTCR-Chimeric-scFv | EVKLEESGGGLVQPGGSMKLSCVASGFTFSNYWMNWVRQSPEKGLEWVAEIRLKSNNYATHY AESVKGRFTISRDDSKSSVYLQMNNLRAEDTGIYYCTRNGNYVDYAMDYWGQGTSVTVSS-GGGGSGGGGSGGGGSGGGGSGGGGSGGGGS-DIVMTQSHKFMSTSVGDRVSITCKASQDVSTAVAWYQQKPGQSPKLLIYWASTRHTGVPDRFT GSGSGTDYTLTISSVQAEDLALYYCQQHYSTPWTFGGGTKLEIK (SEQ ID NO:64) |
| VTCR-Chimeric-scFv-DS | EVKLEESGGGLVQPGGSMKLSCVASGFTFSNYWMNWVRQSPEKCLEWVAEIRLKSNNYATHY AESVKGRFTISRDDSKSSVYLQMNNLRAEDTGIYYCTRNGNYVDYAMDYWGQGTSVTVSS-GGGGSGGGGSGGGGSGGGGSGGGGSGGGGS-DIVMTQSHKFMSTSVGDRVSITCKASQDVSTAVAWYQQKPGQSPKLLIYWASTRHTGVPDRFT GSGSGTDYTLTISSVQAEDLALYYCQQHYSTPWTFGCGTKLEIK (SEQ ID NO:65) |
| VTCR-NKTT120-scFv | EVQLVESGGGLVQPGGSLRLSCVASGFTFSNYWMNWVRQAPGKGLEWVAEIRLKSNNYATHY AESVKGRFTISRDDSKNTVYLQMNSLRAEDTAVYYCTRNGNYVDYAMDYWGQGTLVTVSS-GGGGSGGGGSGGGGSGGGGSGGGGSGGGGS-DIQMTQSPSSLSASVGDRVTITCKASQDVSTAVAWYQQKPGQAPRLLIYWASTRHTGVPSRFSG SGSGTDFTLTISSLQPEDFALYYCQQHYSTPWTFGQGTKLEIK (SEQ ID NO:66) |
| VTCR-NKTT120-scFv-DS | EVQLVESGGGLVQPGGSLRLSCVASGFTFSNYWMNWVRQAPGKCLEWVAEIRLKSNNYATHY AESVKGRFTISRDDSKNTVYLQMNSLRAEDTAVYYCTRNGNYVDYAMDYWGQGTLVTVSS-GGGGSGGGGSGGGGSGGGGSGGGGSGGGGS-DIQMTQSPSSLSASVGDRVTITCKASQDVSTAVAWYQQKPGQAPRLLIYWASTRHTGVPSRFSG SGSGTDFTLTISSLQPEDFALYYCQQHYSTPWTFGCGTKLEIK (SEQ ID NO:67) |
| VTCR-H1L1-scFv | EVQLVESGGGLVQPGGSLKLSCAASGFTFSNYWMNWVRQAPGKGLEWVAEIRLKSNNYATHY AESVKGRFTISRDDSKNTAYLQMNSLKTEDTAVYYCTRNGNYVDYAMDYWGQGTLVTVSS-GGGGSGGGGSGGGGSGGGGSGGGGSGGGGS-DIQMTQSPSSLSASVGDRVTITCRASQDVSTAVAWYQQKPGKAPKLLIYWASTRHTGVPSRFSG SGSGTDYTLTISSLQPEDFATYYCQQHYSTPWTFGGGTKVEIK (SEQ ID NO:68) |
| VTCR-H1L1-scFv-DS | EVQLVESGGGLVQPGGSLKLSCAASGFTFSNYWMNWVRQAPGKCLEWVAEIRLKSNNYATHY AESVKGRFTISRDDSKNTAYLQMNSLKTEDTAVYYCTRNGNYVDYAMDYWGQGTLVTVSS-GGGGSGGGGSGGGGSGGGGSGGGGSGGGGS-DIQMTQSPSSLSASVGDRVTITCRASQDVSTAVAWYQQKPGKAPKLLIYWASTRHTGVPSRFSG SGSGTDYTLTISSLQPEDFATYYCQQHYSTPWTFGCGTKVEIK (SEQ ID NO:69) |

TABLE 10

Sequences of anti-BBGL1 and anti-BBGL-2 constructs with NKT cell effector function. CLK = kappa CL domain, CLL = lambda CL domain

| | | | | |
|---|---|---|---|---|
| Construct | Sequence of Chain 1 | Sequence of Chain 2 | Sequence of Chain 3 | Sequence of Chain 4 |
| VIT-206 | (BBGL1-VLL or BBGL1-VLK) - (CLK or CLL)-GGGGSGGGGSGG GGSGGGGS(=4X SEQ ID NO: 92) -(anti-Anti-Vα24Jα18 TCR scFv) | Same as Chain 1 | (BBGL1-VH)-(V-IGG4-A or V-IGG2 or V-IGG2-C) | Same as Chain 3 |
| VIT-207 | (BBGL2-VLL or BBGL2-VLK) - (CLK or CLL)-GGGGSGGGGSGG GGSGGGGS(=4X SEQ ID NO: 92)-(anti-Anti-Vα24Jα18 TCR scFv) | Same as Chain 1 | (BBGL2-VH)-(V-IGG4-A or V-IGG2 or V-IGG2-C) | Same as Chain 3 |
| VIT-208 | (BBGL1-VLL or BBGL1-VLK) - (CLK or CLL)-GGGGSGGGGSGG GGSGGGGS(=4X SEQ ID NO: 92)-(anti-Anti-Vα24Jα18 TCR scFv) | (BBGL2-VLL or BBGL2-VLK) - (CLK or CLL)-GGGGSGGGGSGG GGSGGGGS(=4X SEQ ID NO: 92)-(anti-Anti-Vα24Jα18 TCR scFv) | (BBGL1-VH)-(V-IGG4-A or V-IGG2-A or V-IGG2-D) | (BBGL2-VH)-(V-IGG4-B or V-IGG2-B or V-IGG2-E) |
| VIT-209 | (BBGL1-VLL or BBGL1-VLK) - (CLK or CLL) | (BBGL2-VLL or BBGL2-VLK) - (CLK or CLL)-GGGGSGGGGSGG GGSGGGGS(=4X SEQ ID NO: 92)-(anti-Anti-Vα24Jα18 TCR scFv) | (BBGL1-VH)-(V-IGG4-A or V-IGG2-A or V-IGG2-D) | (BBGL2-VH)-(V-IGG4-B or V-IGG2-B or V-IGG2-E) |
| VIT210 | (BBGL1-VLL or BBGL1-VLK) - (CLK or CLL)-GGGGSGGGGSGG GGSGGGGS(=4X SEQ ID NO: 92)-(anti-Anti-Vα24Jα18 TCR scFv) | (BBGL2-VLL or BBGL2-VLK) - (CLK or CLL) | (BBGL1-VH)-(V-IGG4-A or V-IGG2-A or V-IGG2-D) | (BBGL2-VH)-(V-IGG4-B or V-IGG2-B or V-IGG2-E) |

### Example 5: Design of Anti-BBGL-1 and Anti-BBGL-2 Antibodies with T Cell, NK Cell, and NKT Cell Engager Effector Functions

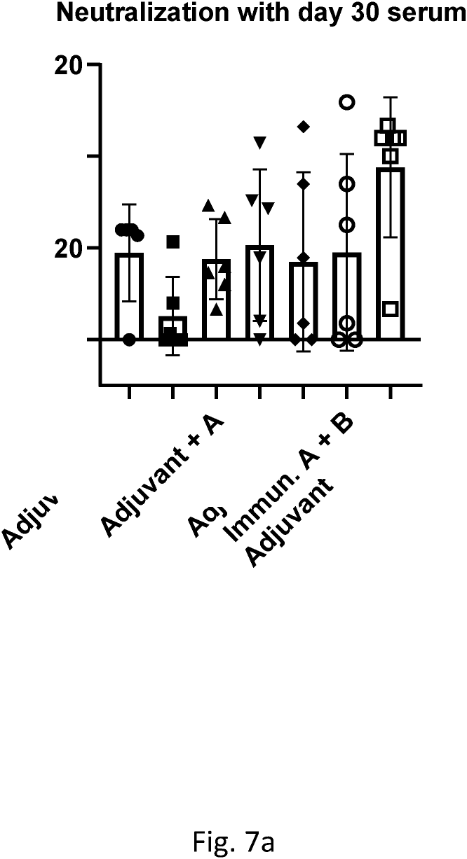

To more specifically activate NKT cells and to a lesser degree T cells and NK cells, novel anti CD3 x CD16/CD56 x BBGL-1/BBGL2 multi-specific antibodies were designed (see FIG. 6). The anti-CD3 and anti-CD16/CD56 moieties are encoded by Fab domains, and the anti-BBGL-1/BBGL-2 moieties are encoded by scFv fragments fused by a GlySer linker (GGGGSGGGGSGGGGS (=3x SEQ ID NO: 92)) at the C terminus of the Light chain. Null Fc fragments are incorporated using modified IgG4 or IgG2 fragments.

TABLE 11

Sequences of anti-BBGL1 and anti-BBGL-2 constructs with T cell, NK cell and NKT cell effector function

| | | | | |
|---|---|---|---|---|
| Construct | Sequence of Chain 1 | Sequence of Chain 2 | Sequence of Chain 3 | Sequence of Chain 4 |
| VIT-216 | (anti-CD3 VLK-CLK)-GGGGSGGGGSGG GGS(=3x SEQ ID NO: 92)-(BBGL1-VH-VLK-scFv or BBGL1-VH-VLL-scFv) | (anti-CD16 or CD56 VLK or VLL)-(CLK or CLL)-GGGGSGGGGSGG GGS(=3x SEQ ID NO: 92)-(BBGL1-VH-VLK-scFv or BBGL1-VH-VLL-scFv) | (anti-CD3-VH)-(V-IGG4-A or V-IGG2-A or V-IGG2-D) | (anti-CD16 or CD56-VH)-(V-IGG4-B or V-IGG2-B or V-IGG2-E) |
| VIT-217 | (anti-CD3 VLK-CLK)-GGGGSGGGGSGG GGS(=3x SEQ ID NO: 92) -(BBGL2-VH-VLK-scFv or BBGL2-VH-VLL-scFv) | (anti-CD16 or CD56 VLK or VLL)-(CLK or CLL)-GGGGSGGGGSGG GGS(=3x SEQ ID NO: 92)-(BBGL2-VH-VLK-scFv or BBGL2-VH-VLL-scFv) | (anti-CD3-VH)-(V-IGG4-A or V-IGG2-A or V-IGG2-D) | (anti-CD16 or CD56-VH)-(V-IGG4-B or V-IGG2-B or V-IGG2-E) |
| VIT-218 | (anti-CD3 VLK-CLK)-GGGGSGGGGSGG GGS(=3x SEQ ID NO: 92) -(BBGL1-VH-VLK-scFv or | (anti-CD16 or CD56 VLK or VLL)-(CLK or CLL)-GGGGSGGGGSGG GGS(=3x SEQ ID NO: 92)-(BBGL2- | (anti-CD3-VH)-(V-IGG4-A or V-IGG2-A or V-IGG2-D) | (anti-CD16 or CD56-VH)-(V-IGG4-B or V-IGG2-B or V-IGG2-E) |
| | BBGL1-VH-VLL-scFv) | VH-VLK-scFv or BBGL2-VH-VLL-scFv) | | |

### Example 6: Design of Anti-BBGL1 and anti-BBGL2 Constructs With Drug Payload Delivery

Site specific addition of drug payloads to the antibody Fc region was devised by analysis of the co-crystal structure of a human IgG1 Fc with the 3-helix bundle of bacterial protein A (PDB structure 5U4Y https://www.rcsb.org/sequence/5U4Y). Computational modelling revealed that A339C would have a stabilizing effect to the structure and S337C or K340C would have a neutral effect to the stability of the Fc domain. A339C was chosen as the site for site specific conjugation.

TABLE 12

IgG4 sequences with engineered free cys for site specific payload conjugation

| | | |
|---|---|---|
| Construct | Sequence | Notes |
| V-IGG4-ADC-A | ASTKGPSVFPLAPCSRSTSESTAALGCLVKDYFPE PVTVSWNSGALTSGVHTFPAVLQSSGLYSLSSVV TVPSSSLGTKTYTCNVDHKPSNTKVDKRVESKYG PPCPPCPAPEAAGGPSVFLFPPKPKDTLMISRTP EVTCVVVDVSQEDPEVQFNWYVDGVEVHNAKT KPREEQFNSTYRVVSVLTVLHQDWLNGKEYKCA VSNKGLPSSIEKTISKCKGQPREPQVYTLPPSQEE MTKNQVSLTCLVKGFYPSDIAVEWESNGQPEN NYKTTPPVLDSDGSFFLYSRLTVDKSRWQEGNV FSCSVMHEALHNHYTQKSLSLSLGK (SEQ ID NO:70) | S228P FALA (F234A, L235A) K322A Naturally contains F405 and R409 A339C |
| V-IGG4-ADC-A-Fc | GGGGSGGGGSGGGGSKYGPPCPPCPAPEAAGG PSVFLFPPKPKDTLMISRTPEVTCVVVDVSQEDP EVQFNWYVDGVEVHNAKTKPREEQFNSTYRVV SVLTVLHQDWLNGKEYKCAVSNKGLPSSIEKTISK CKGQPREPQVYTLPPSQEEMTKNQVSLTCLVKG FYPSDIAVEWESNGQPENNYKTTPPVLDSDGSFF LYSRLTVDKSRWQEGNVFSCSVMHEALHNHYT QKSLSLSLGK (SEQ ID NO:71) | Same as above, but with linker and fc only |
| V-IGG4-ADC-B | ASTKGPSVFPLAPCSRSTSESTAALGCLVKDYFPE PVTVSWNSGALTSGVHTFPAVLQSSGLYSLSSVV TVPSSSLGTKTYTCNVDHKPSNTKVDKRVESKYG PPCPPCPAPEAAGGPSVFLFPPKPKDTLMISRTP | S228P FALA (F234A, L235A) |
| | EVTCVVVDVSQEDPEVQFNWYVDGVEVHNAKT KPREEQFNSTYRVVSVLTVLHQDWLNGKEYKCA VSNKGLPSSIEKTISKCKGQPREPQVYTLPPSQEE MTKNQVSLTCLVKGFYPSDIAVEWESNGQPEN NYKTTPPVLDSDGSFLLYSKLTVDKSRWQEGNVF SCSVMHEALHNHYTQKSLSLSLGK (SEQ ID NO:72) | K322A F405L, R409K A339C |
| V-IGG4-ADC-B-Fc | GGGGSGGGGSGGGGSKYGPPCPPCPAPEAAGG PSVFLFPPKPKDTLMISRTPEVTCVVVDVSQEDP EVQFNWYVDGVEVHNAKTKPREEQFNSTYRVV SVLTVLHQDWLNGKEYKCAVSNKGLPSSIEKTISK CKGQPREPQVYTLPPSQEEMTKNQVSLTCLVKG FYPSDIAVEWESNGQPENNYKTTPPVLDSDGSFL LYSKLTVDKSRWQEGNVFSCSVMHEALHNHYT QKSLSLSLGK (SEQ ID NO:73) | Same as above, but with linker and fc only |

Anti-BBGL1 and anti-BBGL-2 constructs with payload conjugation sites were designed and are shown in Table 13.

TABLE 13

Sequences of anti-BBGL1 and anti-BBGL2 constructs with free cys for payload conjugation. CLK = kappa CL domain, CLL = lambda CL domain

| | | | | |
|---|---|---|---|---|
| Construct | Sequence of Chain 1 | Sequence of Chain 2 | Sequence of Chain 3 | Sequence of Chain 4 |
| VIT-203 | (BBGL1-VLL or BBGL1-VLK) - (CLK or CLL) | Same as Chain 1 | (BBGL1-VH) - (V-IGG4-ADC-A) | Same as Chain 3 |
| VIT-204 | (BBGL2-VLL or BBGL2-VLK) - (CLK or CLL) | Same as Chain 1 | (BBGL2-VH) - (V-IGG4-ADC-A) | Same as Chain 3 |
| VIT-205 | (BBGL1-VLL or BBGL1-VLK) - (CLK or CLL) | (BBGL2-VLL or BBGL2-VLK) - (CLK or CLL) | (BBGL1-VH) - (V-IGG4-ADC-A) | (BBGL2-VH) -(V-IGG4-ADC-B) |
| VIT-203sc | (BBGL1-VH-VLK-scFv or BBGL1-VH-VLL-scFv) - (V-IGG4-ADC-A-Fc) | Same as Chain 1 | | |
| VIT-204sc | (BBGL2-VH-VLK-scFv or BBGL2-VH- | Same as Chain 1 | | |
| | VLL-scFv)- (V-IGG4-ADC-A-Fc) | | | |
| VIT-205sc | (BBGL1-VH-VLK-scFv or BBGL1-VH-VLL-scFv) - (V-IGG4-ADC-A-Fc) | (BBGL2-VH-VLK-scFv or BBGL2-VH-VLL-scFv)- (V-IGG4-ADC-B-Fc) | | |

### Example 7. Fusion Proteins With IgM Constant Regions

IgM molecules have robust Fc effector functions, particularly with CDC. IgM molecules naturally homodimerize and then covalently associate into pentamers or hexamers. IgM do not contain hinge regions like IgG molecules and instead contain an extra CH domain (CH1-CH2-CH3-CH4). The homodimeric heavy chains come together at the CH2 and CH4 domains. Based on visual analyses of the crystal structure of the murine IgM CH2 domain (pdb 4JVU), the crystal structure of the murine IgM CH4 domain (pdb 4JVW), and a sequence alignment of the human IgM CH2 and CH4 sequences with the homologous mouse sequences, mutations were designed to induce IgM heavy chain heterodimerization by inducing charge differences at the homodimerization interfaces.

Sequence of human IgM constant region, numbered residues 1-453, where positions 131, 135, 354 and 385 are underlined by uniprot (https://www_uniprot.org/uniprot/P01871):

GSASAPTLFPLVSCENSPSDTSSVAVGCLAQDFLPDSITFSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSK LICQ ATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVT GFSPADVFVQWMQRGQPLSPEKYVTSAPMPE PQAPGRYFAHSILTV
SEEEWNTGETYTCVVAHEALPNRVTERTVDKSTGKPTLYNVSLVMSDTAG
TCY (SEQ ID NO:90)

Sequence of IgM CH2-CH3-CH4 which can used for fusing to antibody fragments (Fab, scFv, VHH, etc) or targeting proteins for adding IgM effector functions (residues 105-453):

V-IGM

VIAELPPKVSVFVPPRDGFFGNPRKSKLICQATGFSPRQIQVSWLREGKQ
VGSGVTTDQVQAEAKESGPTTYKVTSTLTIKESDWLGQSMFTCRVDHRGL
TFQQNASSMCVPDQDTAIRVFAIPPSFASIFLTKSTKLTCLVTDLTTYDS
VTISWTRQNGEAVKTHTNISESHPNATFSAVGEASICEDDWNSGERFTCT
VTHTDLPSPLKQTISRPKGVALHRPDVYLLPPAREQLNLRESATITCLVT
GFSPADVFVQWMQRGQPLSPEKYVTSAPMPEPQAPGRYFAHSILTVSEEE
WNTGETYTCVVAHEALPNRVTERTVDKSTGKPTLYNVSLVMSDTAGTCY
(SEQID NO:91)

Based on the structural analysis, the underlined residues K131 and Q135 were found to be in close proximity in the CH2:CH2 interface, and residues T354 and E385 were found to be in close proximity in the CH4:CH4 interface. The following mutations were made to alter the charge pattern in V-IGM-A and V-IGM-B to induce heterodimer formation of A:B and repel the formations of A:A or B:B.

TABLE 14

IgM constant region mutations to induce heavy chain heterodimer formation

| Position | Wildtype residue in V-IGM | V-IGM-A | V-IGM-B |
|---|---|---|---|
| 131 | K | K/R/H | D/E |
| 135 | Q | K/R/H | D/E |
| 354 | T | D/E | K/R/H |
| 385 | E | D/E | K/R/H |

### Example 8. Synthesis of BbGL1, BbGL2, BbGL1-Biotin, and BbGL2-Biotin

### BbGL1

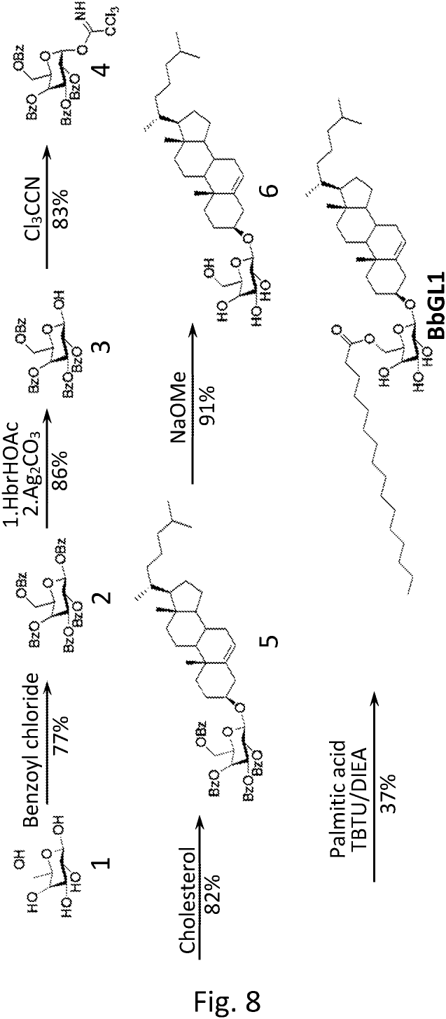

See FIG. 8

### 1,2,3,4,6-penta-O-benzoyl-D-galactose (2)

D-galactose (1.50 g, 8.32 mmol) was suspended in dry pyridine (25 mL). Benzoyl chloride (7.7 mL, 66 mmol) was added dropwise at 0° C. and the mixture was stirred overnight while warming to room temperature. The solvent was evaporated and the mixture was redissolved in dichloromethane (50 mL), washed with sat. NaHCO₃ (2×20 mL) and sat. NaCl (2×20 mL) and dried over MgSO₄. The solvent was evaporated and the product was purified by flash chromatography on silica gel using a gradient of 0-30% ethyl acetate in hexanes as the eluent. Yield: 4.47 g (77%).

¹H-NMR (300 MHz, CDCl₃): δ (ppm) 8.11 (m, 4H), 7.96 (m, 2H), 7.84 (m, 4H), 7.65 (m, 2H), 7.57-7.23 (m, 13H), 6.95 (d, J = 3.6 Hz, 1H), 6.19 (dd, J = 0.9 Hz, 3.4 Hz, 1H), 6.11 (d, J = 3.2 Hz, 1H), 6.04 (d, J=3.5 Hz, 1H) 4.83 (t, J=6.7 Hz, 1H), 4.63 (dd, J=6.4 Hz, 11.4 Hz, 1H), 4.42 (dd, J=7.0 Hz, 11.2 Hz, 1H).

LC-MS (ESI): Calculated for C₄₁H₃₂O₁₁: 700.19; Found: 723.35 (M + Na⁺). Purity: > 99%

### 2,3,4,6-tetra-O-benzoyl-D-galactose (3)

1,2,3,4,6-penta-O-benzoyl-D-galactose (2, 4.47 g, 6.38 mmol) was dissolved in dry dichloromethane (25 mL). 33% HBr in acetic acid (12.5 mL, 51 mmol) was added dropwise at 0° C. and the mixture was stirred overnight while warming to room temperature. The mixture was diluted with dichloromethane (50 mL), washed with sat. NaHCO₃ (3×50 mL) and sat. NaCl (2×25 mL) and dried over MgSO₄. The solvent was evaporated and the resulting white solid was dissolved in a mixture of acetone (20 mL) and water (2 mL). Silver carbonate (2.29 g, 8.29 mmol) was added and the mixture was stirred at room temperature for 30 min. Solids were filtered off over celite and the solvent was evaporated. The product was purified by flash chromatography on silica gel using a gradient of 0-100% ethyl acetate in hexanes as the eluent. Yield: 3.27 g (86%).

¹H-NMR (300 MHz, CDCl₃): δ (ppm) 8.12-7.95 (m, 6H), 7.79 (m, 10H), 7.28-7.21 (m, 2H), 6.07 (m, 2H), 5.85 (t, J = 3.6 Hz, 1H), 5.77-5.67 (m, 1H) 4.88 (t, J=6.5 Hz, 1H), 4.62 (dd, J=6.6 Hz, 11.4 Hz, 1H), 4.40 (dd, J=6.5 Hz, 11.2 Hz, 1H), 2.97 (dd, J=1.2 Hz, 3.8 Hz, 1H).

LC-MS (ESI): Calculated for C₃₄H₂₈O₁₀: 596.17; Found: 619.23 (M + Na⁺). Purity: 98%.

### 2,3,4,6-tetra-O-benzoyl-D-galactopyranosyl Trichloroacetimidate (4)

2,3,4,6-tetra-O-benzoyl-D-galactose (3, 3.27 g, 5.48 mmol) was dissolved in dry dichloromethane (15 mL). The mixture was cooled to 0° C. and trichloroacetonitrile (2.75 mL, 27 mmol) was added, followed by 1,8-Diazabicyclo[5.4.0]undec-7-ene (0.41 mL, 2.74 mmol). The mixture was stirred at 0° C. for 30 min, concentrated by rotary evaporation, and purified by flash chromatography on silica gel using a gradient of 0-30% ethyl acetate in hexanes as the eluent. Yield: 3.36 g (83%).

¹H-NMR (300 MHz, CDCl₃): δ (ppm) 8.63 (s, 1H), 8.09 (m, 2H), 7.97 (m, 4H), 7.81 (m, 2H), 7.64 (m, 1H), 7.58-7.26 (m, 11H), 6.92 (d, J = 3.8 Hz, 1H), 6.16 (m, 1H), 6.06 (d, J = 3.1 Hz, 1H), 5.98 (d, J=3.6 Hz, 1H) 4.87 (t, J=6.6 Hz, 1H), 4.62 (dd, J=6.8 Hz, 11.4 Hz, 1H), 4.44 (dd, J=6.0 Hz, 11.4 Hz, 1H).

LC-MS (ESI): Calculated for C₃₆H₂₈Cl₃NOₗₒ: 739.08; Found: 762.18 (M + Na⁺). Purity: 91%.

### Cholesteryl 2,3,4,6-tetra-O-benzoyl-β-D-galactopyranoside (5)

2,3,4,6-tetra-O-benzoyl-D-galactopyranosyl trichloroacetimidate (4, 3.36 g, 4.53 mmol) and cholesterol (1.93 g, 4.99 mmol) were dissolved in dry dichloromethane (45 mL). 4 Å molecular sieve (powder, 1 g) was added and the mixture was stirred for 10 min. A solution of trimethylsilyl trifluoromethanesulfonate (0.16 mL, 0.91 mmol) in dichloromethane (5 mL) was added dropwise. The mixture was stirred at room temperature for 1 h, quenched with triethylamine, and filtered over celite. The solvent was evaporated and the product was purified by flash chromatography on silica gel using a gradient of 0-30% ethyl acetate in hexanes as the eluent. Yield: 3.57 g (82%).

¹H-NMR (300 MHz, CDCl₃): δ (ppm) 8.10 (m, 2H), 8.03 (m, 2H), 7.96 (m, 2H), 7.79 (m, 2H), 7.65-7.35 (m, 12H), 5.97 (d, J = 3.5 Hz, 1H), 5.77 (dd, J=8.0 Hz, 10.4 Hz, 1H), 5.58 (dd, J = 3.5 Hz, 10.4 Hz, 1H), 5.22 (d, J=4.7 Hz, 1H) 4.90 (t, J=8.1 Hz, 1H), 4.67 (dd, J=6.9 Hz, 11.2 Hz, 1H), 4.42 (dd, J=6.4 Hz, 11.2 Hz, 1H), 4.31 (t, J=6.7 Hz, 1H), 3.55 (s, br, 1H), 2.18 (d, J=7.7 Hz, 2H), 2.05-0.85 (m, 38H), 0.65 (s, 3H).

### Cholesteryl-β-D-galactopyranoside (6)

Cholesteryl 2,3,4,6-tetra-O-benzoyl-β-D-galactopyranoside (5, 3.57 g, 3.70 mmol) was dissolved in a mixture of dry THF (50 mL) and dry methanol (50 mL). Sodium methoxide (200 mg, 3.70 mmol) was added and the mixture was stirred at room temperature for 2.5 h. The reaction was quenched with acetic acid (0.21 mL, 3.70 mmol) and the solvent was evaporated. The product was purified by flash chromatography on silica gel using a gradient of 0-20% methanol in dichloromethane as the eluent. Yield: 1.84 g (91%).

¹H-NMR (300 MHz, DMSO): δ (ppm) 5.32 (d, J=3.9 Hz, 1H) 4.71 (d, J=4.0 Hz, 1H), 4.64 (d, J=4.8 Hz, 1H), 4.51 (t, J=5.6 Hz, 1H), 4.29 (d, J=4.6 Hz, 1H), 4.17 (d, J=7.0 Hz, 1H), 3.60 (s, br, 1H), 2.45-0.80 (m, 39H), 0.65 (s, 3H).

LC-MS (ESI): Calculated for C₃₃H₅₆O₆: 548.41; Found: 571.41 (M + Na⁺).

### BbGL1

Palmitic acid (561 mg, 2.19 mmol) and TBTU (702 mg, 2.19 mmol) were dissolved in dry pyridine (30 mL). N,N-diisopropylethylamine (0.64 mL, 3.64 mmol) was added and the mixture was stirred at room temperature for 30 min. A solution of cholesteryl-β-D-galactopyranoside (6, 1.00 g, 1.82 mmol) in dry pyridine (10 mL) was added and the mixture was stirred at room temperature for 3 days. The solvent was evaporated and the product was purified by flash chromatography on silica gel using a gradient of 0-70% ethyl acetate in hexanes as the eluent. The purified product was dissolved in diethyl ether (50 mL) and washed with sat. NaHCO₃ (2×20 mL) and sat. NaCl (2×20 mL). The combined aqueous phases were back-extracted with diethyl ether. The combined organic phases were dried over MgSO₄ and the solvent was evaporated. Yield: 523 mg (37%).

¹H-NMR (300 MHz CDCl₃): δ (ppm) 5.35 (d, J=4.8 Hz, 1H), 4.33 (m, 3H), 3.88 (s, br, 1H), 3.62 (m, 4H), 2.82 (s, br, 1H), 2.68 (s, br, 1H), 2.55 (s, br, 1H), 2.32 (m, 4H), 2.06-0.80 (m, 70H), 0.68 (s, 3H).

MS (ESI): Calculated for C₄₉H₈₆O₇: 786.64; Found: 809.59 (M + Na⁺)

### BbGL1-Biotin

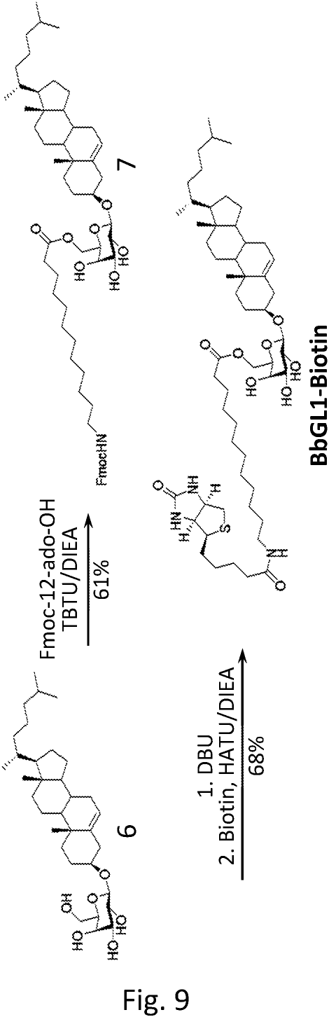

See FIG. 9

### 6-(9H-Fluoren-9-ylmethoxycarbonylamino)-dodecanoyl]-1-cholesteryl-β-D-galactopyranoside (7)

12-(9H-Fluoren-9-ylmethoxycarbonylamino)-dodecanoic acid (250 mg, 0.57 mmol) and TBTU (183 mg, 0.57 mmol) were dissolved in dry pyridine (6 mL). N,N-diisopropylethylamine (0.17 mL, 0.95 mmol) was added and the mixture was stirred at room temperature for 1 h. A solution of cholesteryl-β-D-galactopyranoside (6, 261 mg, 0.47 mmol) in dry pyridine (10 mL) was added and the mixture was stirred at room temperature for 2 days. The solvent was evaporated and the product was purified by flash chromatography on silica gel using a gradient of 0-75% ethyl acetate in hexanes as the eluent. Yield: 283 mg (61%).

¹H-NMR (300 MHz CDCl₃): δ (ppm) 7.76 (d, J=7.4 Hz, 2H), 7.59 (d, J=7.5 Hz, 1H), 7.40 (t, J=7.3 Hz, 1H), 7.31 (t, J=7.6 Hz, 1H), 5.33 (s, br, 1H), 4.82 (s, br, 1H),4.48-4.16 (m, 6H), 3.90 (s, br, 1H), 3.72-3.46 (m, 4H), 3.18 (q, J=6.5 Hz, 2H), 2.31 (m, 5H), 1.70-0.82 (m, 70H), 0.67 (s, 3H).

LC-MS (ESI): Calculated for C₆₀H₈₉NO₉: 967.65; Found: 990.62 (M + Na⁺).

### BbGL1-Biotin

Compound 7 (40 mg, 0.041 mmol) was dissolved in dry DMF (1 mL). 1,8-Diazabicyclo[5.4.0]undec-7-ene (7 µL, 0.045 mmol) was added at 0° C. and the mixture was stirred at 0° C. for 25 min. Biotin-NHS was added, the mixture was warmed to room temperature, and stirred for 2 h. 5 µL extra 1,8-Diazabicyclo[5.4.0]undec-7-ene was added and the mixture was stirred for another 30 min. The reaction mixture was diluted with THF and purified by chromatography on Bio-Beads S-X3 using THF as the eluent. Yield: 27 mg (68%).

¹H-NMR (300 MHz DMSO): δ (ppm) 6.39 (d, J=17.8 Hz, 1H), 5.32 (s, br, 1H), 4.81 (t, J=4.4 Hz, 2H), 4.61 (d, J=4.5 Hz, 1h), 4.34-3.92 (m, 5H), 3.58 (s, br, 2H), 3.14-2.76 (m, 2H), 2.40-1.70 (m, 9H), 1.65-0.80 (m, 73H), 0.65 (s, 3H).

MS (ESI): Calculated for C₅₅H₉₃N₃O₉S: 971.66; Found: 972.73 (M + H+)

### BbGL2

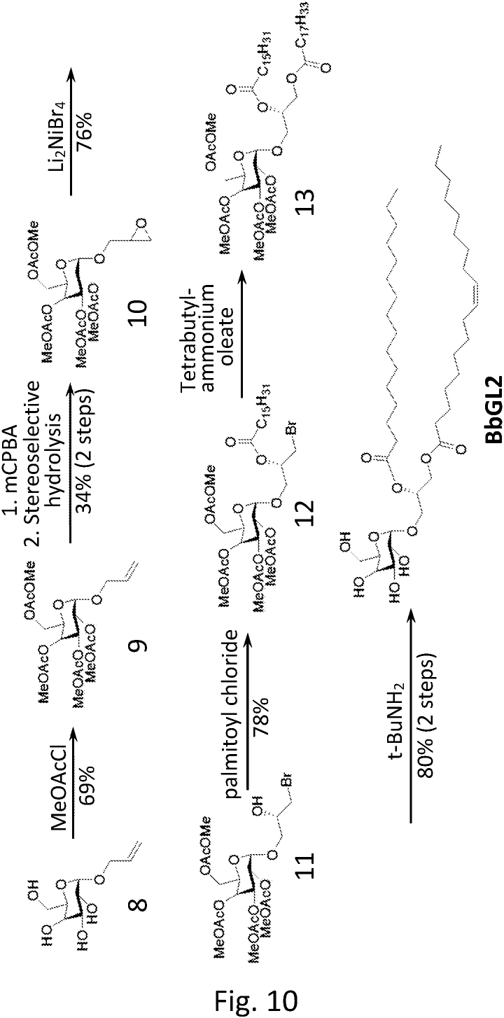

See FIG. 10

### Allyl 2,3,4,6-tetra-O-methoxyacetyl-α-D-galactopyranoside (9)

Allyl α-D-galactopyranoside (1.50 g, 6.81 mmol) was dissolved in dry pyridine (45 mL). Methoxyacetyl chloride (3.4 mL, 37.2 mmol) was added dropwise at 0° C. The mixture was stirred for 2 h while warming to room temperature. Ethyl acetate (150 mL) was added and the mixture was washed with water (2×150 mL), sat. CuSO₄ (3×50 mL), sat. NaHCO₃ (1×50 mL), and sat. NaCl (1×50 mL). The mixture was dried over MgSO₄ and the solvent was evaporated. The product was purified by flash chromatography on silica gel using a gradient of 0-75% ethyl acetate in hexanes as the eluent. Yield: 2.40 g (69%).

¹H-NMR (300 MHz CDCl₃): δ (ppm) 5.86 (m, 1H), 5.56-5.49 (m, 2H), 5.35-5.13 (m, 5H), 4.37-3.83 (m, 15H) 3.48-3.41 (m, 12H).

LC-MS (ESI): Calculated for C₂₁H₃₂O₁₄: 508.47; Found: 531.26 (M + Na⁺).

### (2′R)-2′,3′-Epoxypropyl 2,3,4,6-tetra-O-methoxyacetyl-α-D-galactopyranoside (10)

Allyl 2,3,4,6-tetra-O-methoxyacetyl-α-D-galactopyranoside (9, 2.40 g, 4.72 mmol) was dissolved in dry dichloromethane (50 mL). m-chloroperoxybenzoic acid (2.04 g, 11.8 mmol) was added and the mixture was stirred at RT for 3 days. The mixture was diluted with dichloromethane (100 mL) and washed with sat. Na₂S₂O₅ (50 mL), sat. NaHCO₃ (50 mL), and water (50 mL). The organic phase was dried over MgSO₄, concentrated by rotary evaporation, and purified by flash chromatography on silica gel using a gradient of 0-75% ethyl acetate in hexanes as the eluent. Yield: 1.66 g (67%).

(S,S)-(+)-N,N′ -Bis(3,5-di-tert-butylsalicylidene)-1,2-cyclohexanediaminocobalt(II) (61 mg, 0.10 mmol) and p-toluenesulfonic acid monohydrate (21.22 g, 0.11 mmol) were dissolved in dichloromethane (1.7 mL) and stirred at room temperature for 30 min. The solvent was evaporated and the mixture was redissolved in dry THF (3 mL) and added to the racemic epoxidation product (2.66 g, 5.07 mmol). The mixture was cooled to 0° C., water (50 µL, 2.79 mmol) was added and the mixture was stirred overnight while warming to room temperature. The solvent was evaporated and the product was purified by flash chromatography on silica gel using a gradient of 0-100% ethyl acetate in hexanes as the eluent. Yield: 1.34 g (50%).

¹H-NMR (300 MHz CDCl₃): δ (ppm) 5.57-5.47 (m, 2H), 5.26-5.18 (m, 2H), 4.37 (t, J=6.7 Hz, 1H), 4.28-3.84 (m, 12H) 3.49-3.40 (m, 12H), 3.18 (m, 1H), 2.82 (t, J=4.5 Hz, 1H), 2.61 (dd, J=2.6 Hz, 4.9 Hz, 1H).

LC-MS (ESI): Calculated for C₂₁H₃₂O₁₅: 524.17; Found: 547.38 (M + Na⁺).

### (2′R)-3′-Bromo-2′-hydroxypropyl 2,3,4,6-tetra-O-methoxyacetyl-α-D-galactopyranoside (11)

Lithium bromide (2 g) and Nickel bromide (2.5 g) were dissolved in dry THF (27.5 mL) and the mixture was stirred at room temperature for 2 days. The solution was allowed to settle and 9.6 mL of the supernatant was added to a solution of (2′R)-2′,3′-Epoxypropyl 2,3,4,6-tetra-O-methoxyacetyl-α-D-galactopyranoside (10, 1.34 g, 2.55 mmol) in dry THF (20 mL). The mixture was stirred for 1 h, diluted with dichloromethane (150 mL), and washed with water (3×50 mL). The organic phase was dried over MgSO₄ and the solvent was evaporated. The product was purified by flash chromatography on silica gel using a gradient of 0-100% ethyl acetate in hexanes as the eluent. Yield: 1.18 g (76%).

¹H-NMR (300 MHz CDCl₃): δ (ppm) 5.57-5.45 (m, 2H), 5.26-5.17 (m, 2H), 4.40 (t, J=6.8 Hz, 1H), 4.28-3.82 (m, 13H), 3.62 (dd, J=6.1 Hz, 10.6 Hz, 1H), 3.55-3.41 (m, 12H), 2.61 (d, J=5.7 Hz, 1H).

LC-MS (ESI): Calculated for C₂₁H₃₃BrO₁₅: 604.10; Found: 627.31 (M + Na⁺).

### (2′R)-3′-Bromo-2′-palmitoyloxypropyl 2,3,4,6-tetra-O-methoxyacetyl-α-D-galactopyranoside (12)

(2′R)-3′-Bromo-2′-hydroxypropyl 2,3,4,6-tetra-O-methoxyacetyl-α-D-galactopyranoside (11, 0.93 g, 1.54 mmol) was dissolved in dry dichloromethane (20 mL). Pyridine (2.48 mL, 30 mmol) was added and the mixture was cooled to 0° C. Palmitoyl chloride was added dropwise and the mixture was stirred at 0° C. for 1 h. The mixture was diluted with dichloromethane (30 mL) and washed with water (20 mL), sat. CuSO₄ (2×20 mL), sat. NaHCO₃ (20 mL), and sat. NaCl. The organic phase was dried over MgSO₄ and concentrated by rotary evaporation. The product was purified by flash chromatography on silica gel using a gradient of 0-50% ethyl acetate in hexanes as the eluent. Yield: 1.02 g (78%).

¹H-NMR (300 MHz CDCl₃): δ (ppm) 5.56 (m, 1H), 5.47 (dd, J=3.3 Hz, 10.1 Hz, 1H), 5.24-5.08 (m, 3H), 4.35-3.48 (m, 18H), 3.55-3.42 (m, 12H), 2.36 (t, J=7.4 Hz, 2H), 1.64 (t, J=7.0 Hz, 2H), 1.38-1.18 (m, 26H), 0.88 (t, J=6.7 Hz, 3H).

LC-MS (ESI): Calculated for C₃₇H₆₃BrO₁₆: 842.33; Found: 865.59 (M + Na⁺).

### 1-O-oleoyl-2-O-palmitoyl-sn-glyceryl 2,3,4,6-tetra-O-methoxyacetyl-α-D-galactopyranoside (13)

Tetra-n-butylammonium hydroxide solution (55% in water, 1.14 mL, 2.42 mmol) was added to a solution of oleic acid (717 mg, 2.54 mmol) in water (3 mL). The mixture was stirred overnight and the solvent was evaporated. The mixture was dried azeotropically with toluene and dissolved in dry DMF (15 mL). (2′R)-3′-Bromo-2′-palmitoyloxypropyl 2,3,4,6-tetra-O-methoxyacetyl-α-D-galactopyranoside (12, 1.02 g, 1.21 mmol) was added and the mixture was stirred at 80° C. for 1 h. Diethyl ether (75 mL) was added and the mixture was washed with water (2×25 mL) and sat. NaCl (25 mL). The organic phase was dried over MgSO₄ and the solvent was evaporated. The product was used in the next step without purification.

### BbGL2

Crude 1-O-oleoyl-2-O-palmitoyl-sn-glyceryl 2,3,4,6-tetra-O-methoxyacetyl-α-D-galactopyranoside (13, 1.26 g, 1.21 mmol) was dissolved in a mixture of chloroform (4.5 mL) and methanol (12 mL). t-butylamine (2.53 mL, 24 mmol) was added at 0° C. The ice bath was removed and the mixture was stirred at room temperature for 2 h. The solvent was evaporated and the mixture was purified by flash chromatography on silica gel using a gradient of 0-5% methanol in dichloromethane as the eluent. Yield: 736 mg (80% over 2 steps).

¹H-NMR (300 MHz CDCl₃): δ (ppm) 5.35 (m, 2H), 5.26 (m, 1H), 4.95 (d, J=3.6 Hz, 1H), 4.38 (dd, J=4.2 Hz, 11.9 Hz, 1H), 4.16-4.08 (m, 2H), 4.00-3.78 (m, 5H), 3.64 (dd, J=6.2 Hz, 10.9 Hz, 1H), 2.32 (m, 4H), 2.01 (m, 4H), 1.61 (m, 4H), 1.40-1.17 (m, 46H), 0.88 (t, J=6.7 Hz, 6H).

MS (ESI): Calculated for C₄₃H₈₀O₁₀: 756.58; Found: 779.69 (M + Na⁺)

### BbGL2-Biotin

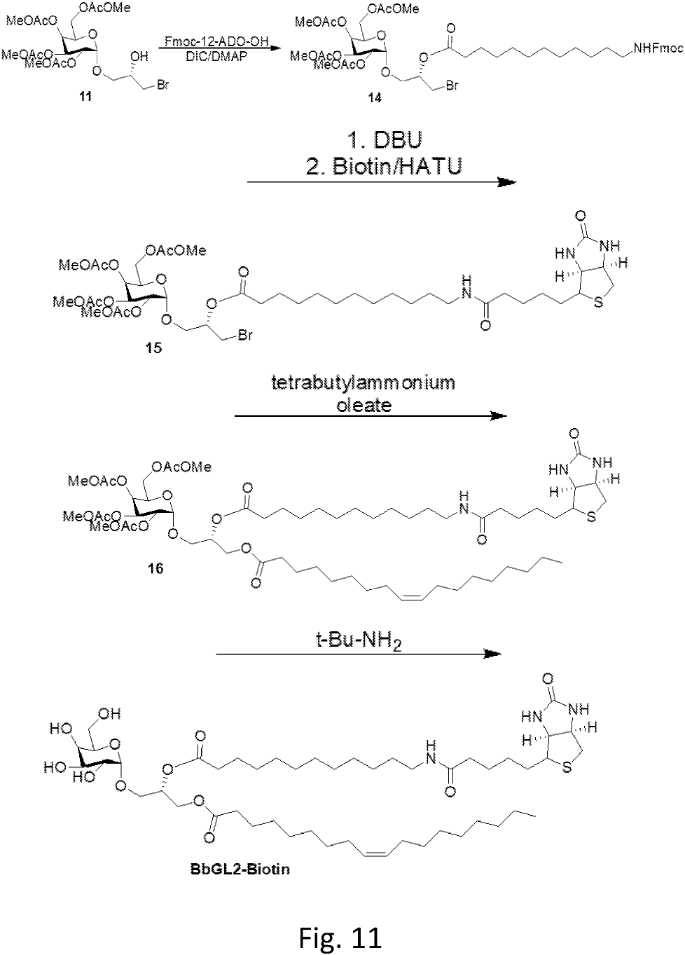

See FIG. 11.

### (2′R)-3′-Bromo-2′-[12-(9H-Fluoren-9-ylmethoxycarbonylamino)-dodecanoyl]-2,3,4,6-tetra-O-methoxyacetyl-α-D-galactopyranoside (14)

(2′R)-3′-Bromo-2′-hydroxypropyl 2,3,4,6-tetra-O-methoxyacetyl-α-D-galactopyranoside (11, 100 mg, 0.17 mmol) and 12-(9H-Fluoren-9-ylmethoxycarbonylamino)-dodecanoic acid (108 mg, 0.25 mmol) were dissolved in dry dichloromethane (1 mL). Diisopropylcarbodiimide (39 µL, 0.25 mmol) and 4-dimethylaminopyridine (30 mg, 0.25 mmol) were added and the mixture was stirred at room temperature for 1.5 h. The product was purified by flash chromatography on silica gel using a gradient of 0-5% methanol in dichloromethane as the eluent. Yield: 132 mg (78%).

¹H-NMR (300 MHz CDCl₃): δ (ppm) 7.76 (d, J=7.4 Hz, 2H), 7.60 (d, J=7.6 Hz, 2H), 7.40 (t, J=7.2 Hz, 2H), 7.31 (dt, J=1.1 Hz, 7.4 Hz, 2H), 5.55 (d, J=3.3 Hz, 1H), 5.47 (dd, J=3.4 Hz, 10 Hz, 1H), 5.28-5.08 (m, 3H), 4.77 (s, br, 1H), 4.40 (d, J=6.9 Hz, 2H), 4.35-3.35 (m, 33H), 3.18 (m, 2H), 2.35 (t, J=7.4 Hz, 2H), 1.64 (m, 2H), 1.50 (m, 2H), 1.28 (m, 2H).

LC-MS (ESI): Calculated for C₄₈H₆₆BrNO₁₈: 1023.35; Found: 1024.70 (M + H⁺).

### (2′R)-3′-Bromo-2′-[12-biotinylamino)-dodecanoyl]-2,3,4,6-tetra-O-methoxyacetyl-a-D-galactopyranoside (15)

(2′R)-3′-Bromo-2′-[12-(9H-Fluoren-9-ylmethoxycarbonylamino)-dodecanoyl]-2,3,4,6-tetra-O-methoxyacetyl-α-D-galactopyranoside (14, 119 mg, 0.12 mmol) was dissolved in dry DMF (2 mL) and cooled to 0° C. 1,8-Diazabicyclo[5.4.0]undec-7-ene (26 µL, 0.17 mmol) was added and the mixture was stirred at 0° C. for 25 min. Biotin (85 mg, 0.35 mmol) and HATU (132 mg, 0.35 mmol) were added and the mixture was stirred for 75 min at 0° C. Ethyl acetate (10 mL) was added and the mixture was washed with water (2×5 mL) and sat. NH₄Cl (2×5 mL). The organic phase was dried over MgSO₄ and the solvent was evaporated. The product was purified by flash chromatography on silica gel using a gradient of 0-10% methanol in dichloromethane as the eluent. Yield: 74 mg (62%).

LC-MS (ESI): Calculated for C₄₃H₇₀BrN₃O₁₈S: 1027.36; Found: 1028.70 (M + H⁺).

### (2′R)-3′-oleoyl-2′-[12-biotinylamino)-dodecanoyl]-2,3,4,6-tetra-O-methoxyacetyl-α-D-galactopyranoside (16)

Tetra-n-butylammonium hydroxide solution (55% in water, 68 µL, 0.14 mmol) was added to a solution of oleic acid (43 mg, 0.15 mmol) in water (0.3 mL). The mixture was stirred overnight and the solvent was evaporated. The mixture was dried azeotropically with toluene and dissolved in dry DMF (1 mL). (2′R)-3′-Bromo-2′-[12-biotinylamino)-dodecanoyl]-2,3,4,6-tetra-O-methoxyacetyl-α-D-galactopyranoside (15, 74 mg, 0.072 mmol) was added and the mixture was stirred at 80° C. for 1 h. Ethyl acetate (10 mL) was added and the mixture was washed with water (2×5 mL) and sat. NaCl (2×5 mL). The organic phase was dried over MgSO₄ and the solvent was evaporated. The product was used in the next step without purification

LC-MS (ESI): Calculated for C₆₁H₁₀₃N₃O₂₀S: 1229.69; Found: 1231.05 (M + H⁺).

### BbGL2-Biotin

Crude (2′R)-3′-oleoyl-2′-[12-biotinylamino)-dodecanoyl]-2,3,4,6-tetra-O-methoxyacetyl-α-D-galactopyranoside (16, 88 mg, 0.072 mmol) was dissolved in a mixture of chloroform (0.3 mL) and methanol (0.8 mL). t-butylamine (0.15 mL, 1.43 mmol) was added at 0° C. The ice bath was removed and the mixture was stirred at room temperature for 75 min. The solvent was evaporated and the mixture was purified by flash chromatography on silica gel using a gradient of 0-20% methanol in dichloromethane as the eluent. Yield: 39 mg (58% over 2 steps).

1H-NMR (300 MHz CDCl₃): δ (ppm) 6.31 (s, br, 1H), 6.21 (s, br, 1H), 5.71 (s, br, 1H), 5.34 (m, 2H), 5.26 (m, 1H), 4.90 (d, J=3.4 Hz, 1H), 4.51 (m, 2H), 4.35 (m, 2H), 4.20-4.09 (m, 2H), 3.95-3.75 (m, 6H), 3.64 (dd, J=5.8 Hz, 10.9 Hz, 2H), 3.36-3.09 (m, 3H), 2.90 (dd, J=4.7 Hz, 12.8 Hz, 1H), 2.75 (d, J=12.8 Hz, 1H), 2.32 (q, J=7.4 Hz, 4H), 2.22 (m, 2H), 2.01 (m, 4H), 1.92-1.18 (m, 49H), 0.88 (t, J=6.7 Hz, 3H).

MS (ESI): Calculated for C₄₉H₈₇N₃O₁₂S: 941.60; Found: 942.89 (M + H⁺)

### Example 9: Preparation of BBGL-1 and BBGL-2 Liposome Vaccines

The following liposome vaccines were generated with the defined compositions:
VIT-GLA: DSPC/DSPG/Cholesterol/BBGL-1 (7:2:1:1 molar ratio)
VIT-GLB: DSPC/DSPG/Cholesterol/BBGL-2 (7:2:1:1 molar ratio)

The liposome vaccines were generated by hydrating and mixing the glycolipids and cholesterol in chloroform and drying overnight at 30 deg C in a rotary evaporator at 120 rpm. The dried mixtures were re-hydrated in aqueous buffer (10 mM phosphate buffer pH7.4) on rotation at 50 deg C for 45 min and then subjected to successive rounds of extrusion through 400 nm, 200 nm and 100 nm membranes to generate 100-150 nm diameter unilammelar vesicles with low polydispersity indices (<0.2). Finished extruded samples are sterile filtered with a 0.22 um syringe filter into vials using aseptic technique in a Laminar Flow Hood. Vials are capped and sealed and stored at 4° C. until further use.

For DLS analysis, samples are diluted into deionized (DI) water.

### Example 10: Stability Profile of Prepared Liposomes VIT-GLA and VIT-GLB by Dynamic Light scattering

The physical stability of the prepared liposomes VIT-GLA and VIT-GLB stored at 4° C. was assessed by dynamic light scattering on a Malvern Zetasizer Nano. Measurements were taken in triplicate and are shown in the tables below.

TABLE 15

Size and polydispersity of VIT-GLA

| Day | Diameter (nm) | Polydispersity Index |
|---|---|---|
| 0 | 157.0 | 0.10 |
| 7 | 157.1 | 0.09 |
| 14 | 156.3 | 0.08 |
| 22 | 157.0 | 0.08 |
| 29 | 157.5 | 0.09 |
| 38 | 158.4 | 0.09 |
| 53 | 156.6 | 0.08 |
| 70 | 155.6 | 0.08 |
| 119 | 158.2 | 0.08 |

TABLE 16

Size and polydispersity of VIT-GLB

| Day | Diameter (nm) | Polydispersity Index |
|---|---|---|
| 0 | 160.4 | 0.19 |
| 4 | 108.2 | 0.04 |
| 11 | 135.0 | 0.06 |
| 19 | 134.2 | 0.07 |
| 26 | 134.4 | 0.09 |
| 35 | 137.7 | 0.07 |
| 50 | 136.0 | 0.06 |
| 67 | 135.7 | 0.07 |
| 116 | 151.2 | 0.17 |

Both VIT-GLA and VIT-GLB were highly stable for greater than 3 months. During the 119 days of testing, VIT-GLA had an average diameter of 155.6-158.4 nm and polydispersity index 0.08-0.10. During the 116 days of testing, VIT-GLB had an average diameter of 108.2-151.2 nm and polydispersity index 0.04-0.19.

### Example 11: Immunization Profile, in Vitro Efficacy, and Safety of an Adjuvanted Lyme Disease Vaccine in Mouse Model

A pilot study in mice was conducted to determine the safety and in vitro efficacy of the BBGL-1 and BBGL-2 liposome vaccines in the absence or presence of the adjuvant QS-21 (Desert King International, San Diego, CA USA). Eighty-four (42 male, 42 female) C3H/HeJ mice (The Jackson Laboratory, Bar Harbor, ME USA), at approximately 7 weeks were used in the study, which is outlined in Table 17a and 17b.

TABLE 17a

Study outline 1

| Description | Group | Size | Vaccine (PO) | Observations | Weights |
|---|---|---|---|---|---|
| Adjuvant Control | 1 | 6M | 1 and 30 | Pre-study, 1 through 60 | Pre-study, 1-7, 15, 22, 29, 36, 43, 50, 57, 60 |
| Adjuvant Control | 1 | 6F | | | |
| Immunization A | 2 | 6M | | | |
| Immunization A | 2 | 6F | | | |
| Adjuvant + Immunization A | 3 | 6M | | | |
| Adjuvant + Immunization A | 3 | 6F | | | |
| Immunization B | 4 | 6M | | | |
| Immunization B | 4 | 6F | | | |
| Adjuvant + Immunization B | 5 | 6M | | | |
| Adjuvant + Immunization B | 5 | 6F | | | |
| Immunization A+B | 6 | 6M | | | |
| Immunization A+B | 6 | 6F | | | |
| Adjuvant + Immunization A+B | 7 | 6M | | | |
| Adjuvant + Immunization A+B | 7 | 6F | | | |

TABLE 17b

Study outline 2

| | | | Blood Collection | | | | | |
|---|---|---|---|---|---|---|---|---|
| | | | | Neutralization | AB response | | | |
| Description | Group | Size | Bio-plex | (4x) | (3x) | Hematology | Chemistry | Necropsy |
| Adjuvant Control | 1 | 6M | Pre-study, 7, 15, 30, 45, 60 (n = 42/collection) | | | Pre-study and 60 (n =42/collection) | | 60 |
| Adjuvant Control | 1 | 6F | | | | | | |
| Immunization A | 2 | 6M | | | | | | |
| Immunization A | 2 | 6F | | | | | | |
| Adjuvant + Immunization A | 3 | 6M | | | | | | |
| Adjuvant + Immunization A | 3 | 6F | | | | | | |
| Immunization B | 4 | 6M | | | | | | |
| Immunization B | 4 | 6F | | | | | | |
| Adjuvant + Immunization B | 5 | 6M | | | | | | |
| Adjuvant + Immunization B | 5 | 6F | | | | | | |
| Immunization A+B | 6 | 6M | | | | | | |
| Immunization A+B | 6 | 6F | | | | | | |
| Adjuvant + Immunization A+B | 7 | 6M | | | | | | |
| Adjuvant + Immunization A+B | 7 | 6F | | | | | | |

7 Group of Mice (6 male and 6 female in each group) were given either 1) adjuvant alone, 2) Immunization A, 3) Adjuvant + Immunization A, 4) Immunization B, 5) Adjuvant + Immunization B, 6) Immunization A + Immunization B, or 7) Adjuvant + Immunization A + Immunization B

Immunization A: DSPC/DSPG/Cholesterol/BBGL-1 (7:2:1:1) (=VIT-GLA, example 9),

Immunization B: DSPC/DSPG/Cholesterol/BBGL-2 (7:2:1:1) (=VIT-GLB, example 9).

Immunization A and Immunization B were administered at 77 µg/mouse doses at day 1 and Day 30. Groups receiving adjuvant received 10 µg QS-21/mouse at Day 1 and Day 30.

The following endpoints were measured during the study:

### 1 Observations and Body Weights

Animals were monitored for clinical signs throughout the study. Body weights were measured at baseline, day 1, 7,15,22,29,36,43,50,57 and 60.

### 2 Clinical Chemistry and Hematology

Blood was collected at baseline and day 60 for clinical chemistry and hematology analysis. For clinical chemistries, whole blood was stored in lithium heparin tubes until analysis.

### 3 Serum Collection

Whole blood was collected into serum separator tubes at baseline and days 7, 15, 30, 45, and 60. Blood was allowed to clot and centrifuged at 1500xg and 10° C. for 15 minutes. Serum was aliquoted and stored at -30° C.

### 5 Neutralization Assay

Serum aliquots were evaluated after 30 days, for neutralizing antibodies against Borrelia burgdorferi, B. mayonii, B. afzelii, and B. garinii using laboratory developed assays.

### 7 Necropsy

At day 60, animals were euthanized, and necropsy was conducted. Brain, lung, heart, liver, kidney, spleen, pancreas, esophagus, stomach, intestine, bone, ovaries, prostate, testes, spinal cord, bladder, lymph nodes were collected for H&E. Samples were stored in 10% Neutral Buffered Formalin.

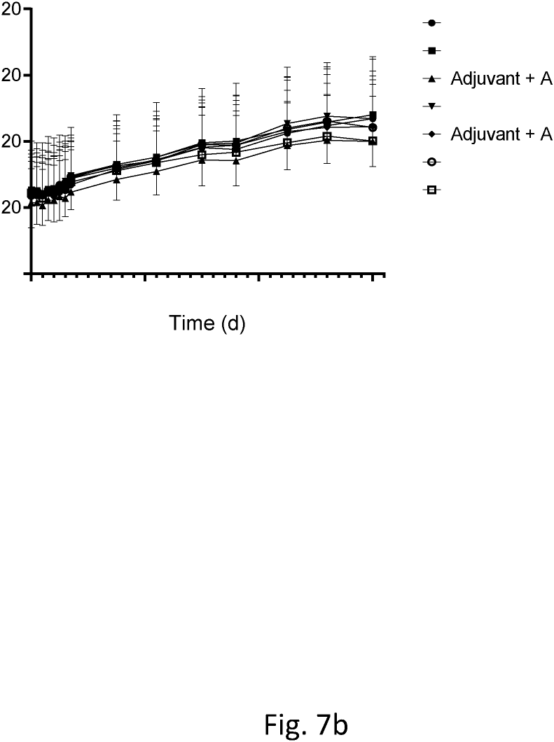

In vitro neutralization of Borrelia burgdorferi from Day 30 assessed with half of the mouse serum samples (n=6 per group) are shown in FIG. 7a. At day 30, Group 1 sera (QS-21 adjuvant alone) showed >15% neutralization activity, whereas Group 7 sera (QS-21 Adjuvant + Immunization A + Immunization B) showed the highest level of neutralization activity (>30%). The average weight of the mice per group (n=12 per group) over the course of the 60 day experiment is shown in FIG. 7b. All groups showed a healthy trend of weight gain, indicating safe delivery of the vaccine test articles.

### Example 12: Immunization Profile, In Vitro Efficacy, and Safety of an Adjuvanted Lyme Disease Vaccine in Mouse Model.

Following the mouse trial conducted as described in Example 11, following analysis will be performed:

### 2 Clinical Chemistry and Hematology

Chemistry analysis of whole blood or plasma will be performed on a Zoetis Vetscan VS2 analyzer with Comprehensive Diagnostic rotors. For hematology, whole blood was stored in EDTA tubes until analysis. Hematology analysis of whole blood will be performed on a Zoetis Vetscan HMS analyzer.

### 4 Immune Titers

Serum aliquots for Immune titer analysis will be tested for IgM, IgG, and IgA.

### 5 Neutralization Assay

Serum aliquots after 60 days will be evaluated for neutralizing antibodies against Borrelia burgdorferi,B. mayonii, B. afzelii, and B. garinii using laboratory developed assays.

### 6 Cytokine Analysis

Cytokine analysis will be performed on serum aliquots using the Bio-Rad Bio-Plex 200 system and Bio-Plex Pro Mouse Cytokine 23-plex Assay kits.

### 7 Necropsy

Brain, lung, heart, liver, kidney, spleen, pancreas, esophagus, stomach, intestine, bone, ovaries, prostate, testes, spinal cord, bladder, lymph nodes will be subjected to H&E.

### Example 13: Use of Biotin-BBGL1 and Biotin-BBGL2 for Measuring Antibody Titers in Lyme Disease Patient Sera

A Meso Scale Discovery Electrochemiluminescence (MSD-ECL) assay was developed using Biotin-BBGL-1 and Biotin-BBGL-2 to measure antibody titers in Lyme Disease patient samples. The assay involved first blocking MSD streptavidin gold plate with 4% BGG (bovine gamma globulin in PBS), washing the plates with PBS and the coating Biotin-BBGL-1 and Biotin-BBGL-2 at concentration of 4 ug/mL (diluted in 4% BGG) for coating for IgG side of plate and at concentration of 1 ug/mL (diluted in 4% BGG) for coating for IgM side of plate. Serum samples (diluted 1:5) were then added to each well, incubated, and washed.

Detection reagents (Sulfo tag anti-lgG Fc or anti-IgM) were then added, incubated, and washed, followed by reading the plates on a Sector Imager (Meso Scale Diagnostic, Rockville, MD USA).

Data from 3 Lyme Disease patients is presented in Table 18 and 19 and shows that this method can detect the presence of anti-BBGL1 and anti-BBGL2 IgG and IgM titers in clinical samples.

TABLE 18

Patients

| # | Gender | Etnicity | Age | ELISA/ Western | Duration Disease | Symptoms |
|---|---|---|---|---|---|---|
| L1 | M | Caucasian | 49 | NA | > 12 months | Joint pain, memory loss |
| L2 | M | Caucasian | 43 | IgG+/ IgM+ | NA | Arthritis |
| L3 | M | Caucasian/ Indian | 36 | IgG+/ IgM+ | 12 months | Arthritis, leg/ foot pain |

TABLE 19

Anti-BBGL1, antiBBGL2 and IgM titers

| | EU | | | | Fold/Baseline | | | |
|---|---|---|---|---|---|---|---|---|
| # | BBGL1 IgM* | BBGL1 IgG** | BBGL2 IgM* | BBGL2 IgG** | BBGL1 IgM* | BBGL1 IgG** | BBGL2 IgM* | BBGL2 IgG** |
| L1 | 4055.50 | 218.50 | 3458.00 | 2720.00 | 21 | 1.3 | 18 | 16 |
| L2 | 390.00 | 143.50 | 344.50 | 619.50 | 2 | 0.9 | 2 | 4 |
| L3 | 1338.00 | 166.00 | 1229.50 | 178.00 | 7 | 1.0 | 6 | 1.1 |

*NC= 189.75
**NC= 166.75

1. A method of preventing, treating and/or alleviating an infectious disease comprising administering to a patient in need thereof a therapeutically effective amount of
a. a vaccine composition comprising a lipid antigen,
b. an antibody or fragment thereof binding a lipid antigen, and/or
c. a pharmaceutical composition comprising an antibody or fragment thereof binding a lipid antigen.

2. A method of preventing, treating and/or alleviating Lyme disease comprising administering to a patient in need thereof a therapeutically effective amount of
a. a vaccine composition comprising a lipid antigen,
b. an antibody or fragment thereof binding a lipid antigen, and/or
c. a pharmaceutical composition comprising an antibody or fragment thereof binding a lipid antigen.

3. A method for producing an antibody or fragment thereof binding a lipid antigen comprising
(i) Providing a vaccine composition comprising a lipid antigen,
(ii) Administering said vaccine composition to an animal or human, to trigger or promote the production of an antibody or fragment thereof binding to said lipid antigen,
(iii) Obtaining said antibody or fragment thereof binding a lipid antigen from said animal or human.

4. A vaccine composition comprising a lipid antigen.

5. The vaccine composition according to claim 4, wherein said lipid antigen is a glycolipid antigen.

6. The vaccine composition according to any of the preceding claims, wherein said lipid antigen is a BBGL-1 and/or BBGL-2 antigen.

7. The vaccine composition according to any of the preceding claims, wherein said antigen is synthetically produced.

8. The vaccine composition according to any of the preceding claims, wherein said antigen is encapsulated in a vesicle comprising at least one lipid layer.

9. The vaccine composition according to any of the preceding claims, wherein said vesicle comprises at least one lipid bilayer.

10. The vaccine composition according to any of the preceding claims, wherein said vesicle is selected from the group consisting of solid lipid nanoparticles (SLNs), emulsions, liposomes, micelles, and bilayer sheets.

11. The vaccine composition according to any of the preceding claims, wherein said vesicle comprises a lipid component selected from the group consisting of phosphatidylcholine, phosphatidylglycerol and cholesterol.

12. The vaccine composition according to any of the preceding claims, wherein said antigen is conjugated to a carrier protein.

13. The vaccine composition according to any of the preceding claims, wherein said antigen is covalently conjugated to a carrier protein.

14. The vaccine composition according to any of the preceding claims, wherein said carrier protein is an immunogenic carrier protein.

15. The vaccine composition according to any of the preceding claims, wherein said carrier protein is selected from the group consisting of bovine serum albumin (BSA) and keyhole limpet haemocyanin (KLH).

16. The vaccine composition according to any of the preceding claims, wherein said vaccine is a multivalent vaccine.

17. The vaccine composition according to any of the preceding claims further comprising one or more pharmaceutically acceptable excipients.

18. The vaccine composition according to any of the preceding claims further comprising an adjuvant.

19. The vaccine composition according to any of the preceding claims, wherein said adjuvant is β-glucan.

20. The vaccine composition according to any of the preceding claims, wherein said vaccine is for an administration form selected among subcutaneous, intradermal, intramuscular, intravenous, oral and nasal.

21. The vaccine composition according to any of the preceding claims, wherein said vaccine is used for prevention, treatment and/or alleviation of Lyme disease.

22. The vaccine composition according to any of the preceding claims, wherein said vaccine is for prevention, treatment and/or alleviation of a condition related to and/or caused by Lyme disease.

23. The vaccine composition according to any of the preceding claims, wherein said condition is selected among Lyme Borreliosis, antibiotic resistant Lyme Borreliosis, synovitis, arthritis, antibiotic refractory arthritis, fatigue, sleep impairment, nerve pain, headaches, memory loss, joint pain and/or depression.

24. The vaccine composition according to any of the preceding claims, comprising one or more liposome(s) comprising one or more lipid antigen(s) embedded in the outer membrane of said liposome(s).

25. The vaccine composition according to claim 24, wherein the one or more lipid antigens are selected among BBGL1, BBGL2 and combinations thereof.

26. The vaccine composition according to claim 24 or 25, wherein the lipids of the liposome comprises one or more of phosphatidylserine (PS), 1,2-dioleyl- sn-glycero-3-phospho-L-serine (DOPS), distearoyl-sn-glycero-3-phosphatidylcholine (DSPC), distearoyl-sn-glycero-3-phosphoethanolamine (DSPE), DSPE-PEG2000, distearoyl-sn-glycero-3-phospho-(1′-rac-glycerol)(DSPG), di-(9Z-octadecanoyl-sn-glycero-3-phosphocholine (DOPC), di-hexadecanoyl-sn-glycero-3-phosphocholine (DPPC), sphingomyelin, di-oleyl-sn-glycero-3-phosphoethanilamine (DOPE) and cholesterol.

27. The vaccine composition according to any of claims 24 - 26, wherein the lipids of the liposome comprise DSPE, DSPG and cholesterol.

28. The vaccine composition according to claim 27, wherein the lipids of the liposome consist of DSPE, DSPG and cholesterol.

29. The vaccine composition according to claim 28, wherein the composition of the liposomes is selected among:
DSPC/DSPG/Cholesterol/BBGL1 in the weight ratio: 7:2:1:1;
DSPC/DSPG/Cholesterol/BBGL2 in the weight ratio: 7:2:1:1; and
DSPC/DSPG/Cholesterol/BBGL1/BBGL2 in the weight ratio: 7:2:1:1:1.

30. The vaccine composition according to any of the claims 24-29, wherein the liposome further comprises a vaccine adjuvant and/or immunostimulant encapsuled inside the liposomes.

31. A method for prevention, treatment and/or alleviation of Lyme disease by administering the vaccine according to any of the preceding claims to a subject in need thereof.

32. The method according to claim 31, wherein said vaccine is administered in an administration form selected among subcutaneous, intradermal, intramuscular, intravenous, oral and nasal.

33. The method according to claim 31, wherein said vaccine is administered in an administration form selected among oral and nasal.

34. A compound selected among
6-[12-(X-amino)-dodecanoyl]-1-cholesteryl-β-D-galactopyranoside, where X is an amino protective group;
Allyl 2,3,4,6-tetra-O-X1-α-D-galactopyranoside, (2′R)-2′,3′-Epoxypropyl 2,3,4,6-tetra-O-X1-α-D-galactopyranoside, (2′R)-3′-Bromo-2′-hydroxypropyl 2,3,4,6-tetra-O-X1-α-D-galactopyranoside, (2′R)-3′-Bromo-2′-palmitoyloxypropyl 2,3,4,6-tetra-O-X1-α-D-galactopyranoside, 1-O-oleoyl-2-O-palmitoyl-sn-glyceryl 2,3,4,6-tetra-O-X1-α-D-galactopyranoside,wherein X1 is an alcohol protection group; and (2′R)-3′-Bromo-2′-[12-(X2-amino)-dodecanoyl]-2,3,4,6-tetra-O-X3-α-D-galactopyranoside, (2′R)-3′-Bromo-2′-[12-biotinylamino)-dodecanoyl]-2,3,4,6-tetra-OX3-α-D-galactopyranoside, (2′R)-3′-oleoyl-2′-[12-biotinylamino)-dodecanoyl]-2,3,4,6-tetra-O-X3-a-D-galactopyranoside, wherein X2 is an amine protective group and X3 is an alcohol protective group.

35. The compound of claim 34 selected among: 6-[12-(9H-Fluoren-9-ylmethoxycarbonylamino)-dodecanoyl]-1-cholesteryl-β-D-galactopyranoside (7), Allyl 2,3,4,6-tetra-O-methoxyacetyl-α-D-galactopyranoside (9), (2′R)-2′,3′-Epoxypropyl 2,3,4,6-tetra-O-methoxyacetyl-α-D-galactopyranoside (10), (2′R)-3′-Bromo-2′-hydroxypropyl 2,3,4,6-tetra-O-methoxyacetyl-α-D-galactopyranoside (11) (2′R)-3′-Bromo-2′-palmitoyloxypropyl 2,3,4,6-tetra-O-methoxyacetyl-α-D-galactopyranoside (12), 1-O-oleoyl-2-O-palmitoyl-sn-glyceryl 2,3,4,6-tetra-O-methoxyacetyl-α-D-galactopyranoside (13), (2′R)-3′-Bromo-2′-[12-(9H-Fluoren-9-ylmethoxycarbonylamino)-dodecanoyl]-2,3,4,6-tetra-O-methoxyacetyl-α-D-galactopyranoside (14), (2′R)-3′-Bromo-2′-[12-biotinylamino)-dodecanoyl]-2,3,4,6-tetra-O-methoxyacetyl-α-D-galactopyranoside (15) and (2′R)-3′-oleoyl-2′-[12-biotinylamino)-dodecanoyl]-2,3,4,6-tetra-O-methoxyacetyl-α-D-galactopyranoside (16).

36. A compound selected among BBGL-1-Biotin and BBGL-2-Biotin.

37. An antibody or fragment thereof binding a lipid antigen.

38. The antibody or fragment thereof according to claim 37, wherein said lipid antigen is a glycolipid antigen.

39. The antibody or fragment thereof according to any of claims 37-38, wherein said lipid antigen is a BBGL-1 and/or BBGL-2 antigen.

40. The antibody or fragment thereof according to any of claims 37-39 binding a BBGL-1 antigen.

41. The antibody or fragment thereof according to any of claims 37-40 binding a BBGL-2 antigen.

42. The antibody or fragment thereof according to any of claims 37-41, binding a BBGL-1 antigen and a BBGL-2 antigen.

43. The antibody or fragment thereof according to any of claims 37-42, wherein said antibody or fragment thereof comprises a homodimeric immunoglobulin or fragment thereof.

44. The antibody or fragment thereof according to any of claims 37-43, wherein said antibody or fragment thereof comprises a heterodimeric immunoglobulin or fragment thereof.

45. The antibody or fragment thereof according to any of claims 37-44, wherein said antibody or fragment thereof is an IgG, IgM IgA, IgD and/or an IgE.

46. The antibody or fragment thereof according to any of claims 37-45, wherein said antibody or fragment thereof is an IgG1, IgG3 and/or IgM.

47. The antibody or fragment thereof according to any of claims 37-46, wherein said antibody or fragment thereof comprises a sequence selected from the group consisting of SEQ ID NOs 1-20.

48. The antibody or fragment thereof according to any of claims 37-47, wherein said antibody or fragment thereof comprises a sequence combination selected from the specific combinations given in Table 3.

49. A method of producing an antibody or fragment thereof according to any of claims 37-48, wherein said antibody or fragment thereof is produced by animal immunization.

50. The method according to claim 49, wherein said animal immunization comprises administering a vaccine according to any of the precedent claims to an animal.

51. The method according to any of claims 49-50, wherein said animal is selected from the group consisting of mouse, rabbit, alpaca and llama.

52. A method of producing an antibody or fragment thereof according to any of claims 49-51, comprising
a. Isolating B cells from a Lyme diseases patient,
b. Isolating B-cell receptors from said B cells,
c. Sequencing said B-cell receptors, and
d. Cloning said B-cell receptors.

53. A method of producing an antibody or fragment thereof according to any of claims 49-52, using a human antibody phage display library.

54. An antibody or fragment thereof obtainable by the method of any of the claims 49-53.

55. The antibody or fragment thereof according to any of claims 37-48, wherein said antibody or fragment thereof further binds a CD3 antigen.

56. The antibody or fragment thereof according to any of claims 37-48 and 55, wherein said antibody or fragment thereof is a T cell engaging antibody.

57. The antibody or fragment thereof according to any of claims 37-48 and 55-56, wherein said antibody or fragment thereof comprises variable light chain CDR sequences of SEQ ID NOs:75-77.

58. The antibody or fragment thereof according to any of claims 37-48 and 55-57, wherein said antibody or fragment thereof comprises variable heavy chain CDR sequences of SEQ ID NOs:78-80.

59. The antibody or fragment thereof according to any of claims 37-48 and 55-58, wherein said antibody or fragment thereof comprises variable light chain CDR sequences of SEQ ID NOs:75-77 and variable heavy chain CDR sequences of SEQ ID NOs:78-80.

60. The antibody or fragment thereof according to any of claims 37-48 and 55-59, wherein said antibody or fragment thereof comprises a sequence selected from the group consisting of SEQ ID NOs 21-38.

61. The antibody or fragment thereof according to any of claims 37-48 and 55-60, wherein said antibody or fragment thereof comprises a sequence selected from the group consisting of SEQ ID NOs 39-57.

62. The antibody or fragment thereof according to any of claims 37-48 and 55-61, wherein said antibody or fragment thereof comprises a sequence combination selected from the specific combinations given in Table 4.

63. The antibody or fragment thereof according to any of claims 37-48 and 55-62, wherein said antibody or fragment thereof further binds a TCR Vα24Jα18 antigen.

64. The antibody or fragment thereof according to any of claims 37-48 and 55-63, wherein said TCR Vα24Jα18 antigen comprises a sequence according to SEQ ID No.: 81, 82 and/or 83.

65. The antibody or fragment thereof according to any of claims 37-48 and 55-64, wherein said antibody is an NKT cell engaging antibody.

66. The antibody or fragment thereof according to any of claims 37-48 and 55-65, wherein said antibody or fragment thereof comprises a variable light chain sequence selected from the group consisting of SEQ ID NOs 58-60.

67. The antibody or fragment thereof according to any of claims 37-48 and 55-66, wherein said antibody or fragment thereof comprises a variable heavy chain sequence selected from the group consisting of SEQ ID NOs 61-63.

68. The antibody or fragment thereof according to any of claims 37-48 and 55-67, wherein said antibody or fragment thereof comprises a variable light chain sequence selected from the group consisting of SEQ ID NOs 58-60 and a variable heavy chain sequence selected from the group consisting of SEQ ID NOs 61-63.

69. The antibody or fragment thereof according to any of claims 37-48 and 55-68, wherein said antibody or fragment thereof comprises a sequence selected from the group consisting of SEQ ID NOs 64-69.

70. The antibody or fragment thereof according to any of claims 37-48 and 55-69, wherein said antibody or fragment thereof comprises a sequence combination selected from the specific combinations given in Table 10.

71. The antibody or fragment thereof according to any of claims 37-48 and 55-70, wherein said antibody or fragment thereof further binds a CD16 and/or a CD56 antigen.

72. The antibody or fragment thereof according to any of claims 37-48 and 55-71, wherein said antibody or fragment thereof further binds a CD16 and a CD56 antigen.

73. The antibody or fragment thereof according to any of claims 37-48 and 55-72, wherein said antibody or fragment thereof specifically activates NKT cells.

74. The antibody or fragment thereof according to any of claims 37-48 and 55-73, wherein said antibody or fragment thereof comprises a sequence combination selected from the specific combinations given in Table 11.

75. The antibody or fragment thereof according to any of claims 37-48 and 55-74, wherein said antibody or fragment thereof engage a cell selected from the group consisting of T cells, NK cells and NKT cells.

76. The antibody or fragment thereof according to any of claims 37-48 and 55-75, wherein said antibody or fragment thereof further comprises a drug payload.

77. The antibody or fragment thereof according to any of claims 37-48 and 55-76, wherein said drug payload is an infrared dye and/or a radionucleotide.

78. The antibody or fragment thereof according to any of claims 37-48 and 55-77, wherein said antibody or fragment thereof comprises a mutation selected from the group consisting of an Ala339Cys, a Ser337Cys and a Lys340Cys mutation.

79. The antibody or fragment thereof according to any of claims 37-48 and 55-77, wherein said antibody or fragment thereof comprises an Ala339Cys mutation.

80. The antibody or fragment thereof according to any of claims 37-48 and 55-79, wherein said drug payload is inserted at a site-specific free Cys residue.

81. The antibody or fragment thereof according to any of claims 37-48 and 55-80, wherein said antibody or fragment thereof comprises a sequence selected from the group consisting of SEQ ID NOs 70-73.

82. The antibody or fragment thereof according to any of claims 37-48 and 55-81, wherein said antibody or fragment thereof comprises a sequence combination selected from the specific combinations given in Table 13.

83. The antibody or fragment thereof according to any of claims 37-48 and 55-82, wherein said antibody or fragment thereof is a scFv.

84. The antibody or fragment thereof according to any of claims 37-48 and 55-83, wherein said antibody or fragment thereof comprises a scFv.

85. The antibody or fragment thereof according to any of claims 37-48 and 55-84, wherein said antibody or fragment thereof provides enhanced ADCC, ADCP and/or CDC.

86. The antibody or fragment thereof according to any of claims 37-48 and 55-85, comprising a first antigen binding site capable of binding to a first antigen and a second antigen binding site capable of binding to a second antigen, wherein said first antigen binding site is comprised in a Fab fragment and said second antigen binding site is comprised in a moiety selected from the group consisting of scFv, antibody fragment and protein moiety, characterized by said moiety being attached to the light chain of said Fab fragment.

87. The antibody or fragment thereof according to any of claim 37-48 and 55-86, wherein said moiety is a scFv.

88. The antibody or fragment thereof according to any of claim 37-48 and 55-87, wherein said moiety is attached to the C-terminus or N-terminus of the light chain of said Fab fragment.

89. The antibody or fragment thereof according to any of claim 37-48 and 55-88, wherein said moiety is attached to the C-terminus of the light chain of said Fab fragment.

90. The antibody or fragment thereof according to any of claim 37-48 and 55-89, wherein the constant region is derived from an IgG or an IgM.

91. The antibody or fragment thereof according to any of claim 37-48 and 55-90, wherein said antibody or fragment thereof comprises a mutation in the IgM constant region, preferably at position 131, 135, 354 and/or 385.

92. The antibody or fragment thereof according to any of claims 37-48 and 55-91, wherein said antibody or fragment thereof comprises a mutation in the IgM constant region selected from the group consisting of K131R, K131H, K131D, K131E, Q135K, Q135R, Q135H, Q135D, Q135E, T354D, T354E, T354K, T354R, T354H, E385D, E385K, E385R and E385H.

93. The antibody or fragment thereof according to any of claims 37-48 and 55-92, wherein said Fab fragment is derived from an IgG.

94. The antibody or fragment thereof according to any of claim 37-48 and 55-93, wherein said Fab fragment is derived from an IgG selected from the group consisting of IgG1, IgG2, IgG3 and IgG4.

95. The antibody or fragment thereof according to any of claim 37-48 and 55-94, wherein said Fab fragment is derived from an IgG2 or an IgG4.

96. The antibody or fragment thereof according to any of claim 37-48 and 55-95, wherein said antibody or fragment thereof comprises a null fc.

97. The antibody or fragment thereof according to any of claim 37-48 and 55-96, wherein said antibody or fragment thereof comprises a null fc, wherein said null fc comprises a Leu234Ala and/or a Leu235Ala and/or a Lys22Ala mutation.

98. The antibody or fragment thereof according to any of claim 37-48 and 55-97, wherein said antibody or fragment thereof comprises a Ser228Pro mutation.

99. The antibody or fragment thereof according to any of claim 37-48 and 55-98, comprising a linker.

100. The antibody or fragment thereof according to any of claim 37-48 and 55-99, wherein said linker is a peptide linker.

101. The antibody or fragment thereof according to any of claim 37-48 and 55-100, wherein said linker is a GlySer linker.

102. The antibody or fragment thereof according to any of claims 37-48 and 55-101, wherein said linker comprises the sequence of SEQ ID NO:71.

103. The antibody or fragment thereof according to any of claim 37-48 and 55-102, wherein said antibody or fragment thereof blocks host cell entry of a pathogen.

104. A pharmaceutical composition comprising an antibody or fragment thereof according to any of claim 37-48 and 55-103, optionally comprising one or more excipients such as diluents, binders or carriers.

105. The pharmaceutical composition according to claim 104, further comprising an adjuvant.

106. The pharmaceutical composition according to claim 105, wherein said adjuvant is β-glucan.

107. The antibody or fragment thereof or pharmaceutical composition according to any of the preceding claims, wherein said antibody or fragment thereof or pharmaceutical composition is for an administration form selected among subcutaneous, intradermal, intramuscular, intravenous, oral and nasal.

108. An isolated nucleic acid molecule encoding an antibody or fragment thereof according to any of the precedent claims.

109. A recombinant vector comprising the nucleic acid molecule of claim 108.

110. A host cell comprising the recombinant vector of claim 109.

111. A method for the production of an antibody or fragment thereof according to any of the precedent claims comprising a step of culturing the host cell according to claim 110, in a culture medium under conditions allowing the expression of the antibody or fragment thereof and separating the antibody or fragment thereof from the culture medium.

112. A method for the production of an antibody or fragment thereof according toany of the precedent claims comprising a synthetic and/or recombinant step.

113. Use of an antibody or fragment thereof or vaccine composition according to any of the preceding claims in a preclinical model.

114. A method of preventing, treating and/or alleviating an infectious disease comprising administering to a patient in need thereof a therapeutically effective amount of the antibody or fragment thereof or pharmaceutical composition according to any of the preceding claims.

115. The method according to any of the preceding claims, wherein said infectious disease is caused by a Borrelia Burgdorferi bacteria sensu lato.

116. The method according to any of the preceding claims, wherein said Borrelia Burgdorferi bacteria sensu lato is B. burgdorferi, B. mayonii, B. afzelii and/or B. garinii.

117. A method of preventing, treating and/or alleviating a condition related to and/or caused by Lyme disease comprising administration to a patient in need thereof a therapeutically effective amount of the antibody or fragment thereof or pharmaceutical composition according to any of the preceding claims.

118. The method according to any of the preceding claims, wherein the condition is selected among Lyme Borreliosis, antibiotic resistant Lyme Borreliosis, synovitis, arthritis, antibiotic refractory arthritis, fatigue, sleep impairment, nerve pain, headaches, memory loss, joint pain and/or depression.

119. A method of preventing, treating and/or alleviating Lyme disease comprising administering to a patient in need thereof a therapeutically effective amount of the antibody or fragment thereof or pharmaceutical composition according to any of the preceding claims.

120. A method of diagnosing Lyme disease comprising administering to a patient in need thereof a therapeutically effective amount of the antibody or fragment thereof or pharmaceutical composition according to any of the preceding claims.

121. A method of diagnosing Lyme disease comprising the steps of
a. obtaining a blood or urine sample from a subject, and
b. testing said blood or urine sample for the presence of a BBGL-1 and/or BBGL-2 biomarker with the antibody or fragment thereof according to any of the preceding claims.

122. A method of selecting and/or identifying a subject that might benefit from treatment with an antibody or fragment thereof or vaccine according to any of the preceding claims comprising the steps of
a. obtaining a blood or urine sample from a subject, and
b. testing said blood or urine sample for the presence of a BBGL-1 and/or BBGL-2 biomarker with the antibody or fragment thereof according to any of the preceding claims.

123. A method of monitoring infectious disease progression and/or disease resolution comprising the steps of
a. obtaining a blood or urine sample from a subject, and
b. testing said blood or urine sample for the presence of a BBGL-1 and/or BBGL-2 biomarker with the antibody or fragment thereof according to any of the preceding claims.

124. A method of detecting BBGL1 and/or BBGL2 specific antibodies in a blood sample using BBGL1-biotin and/or BBGL2-Biotin.

125. A method of in vivo imaging of bacterial infections comprising use of an antibody or fragment thereof according to any of the precedent claims.

126. The antibody or fragment thereof according to any of the precedent claims for use in an imaging technique for monitoring infectious disease progression/resolution.

127. A diagnostic kit comprising an antibody or fragment thereof according to any of the precedent claims and instructions for use.

128. A kit for screening comprising an antibody or fragment thereof according to any of the precedent claims and instructions for use.

129. An antibody or fragment thereof, preferably according to any of the preceding claims, capable of binding a TCR Vα24Jα18 antigen, wherein said antibody or fragment thereof is capable of binding an additional antigen, such as a lipid antigen, allowing NKT cells to be re-directed to said additional antigen.

130. An antibody or fragment thereof, preferably according to any of the preceding claims, capable of binding a CD3 antigen, and wherein said antibody or fragment further is capable of binding a CD16 and/or a CD56 antigen; and preferably wherein said antibody or fragment thereof is capable of binding an additional antigen, such as a lipid antigen, allowing NKT cells to be re-directed to said additional antigen.

131. An antibody or fragment thereof, preferably according to any of the preceding claims, capable of binding a TCR Vα24Jα18 antigen, wherein said antibody is an NKT cell engaging antibody,
wherein said antibody or fragment thereof further is capable of binding CD3 antigen, and wherein said antibody or fragment thereof further is capable of binding a CD16 and/or a CD56 antigen.

132. The antibody or fragment thereof according to any of the preceding claims, wherein said TCR Vα24Jα18 antigen comprises a sequence according to SEQ ID NO.: 81, 82 and/or 83.

133. The antibody or fragment thereof according to any of the preceding claims, wherein said antibody or fragment thereof comprises
a. a variable light chain sequence comprising at least one of the CDR regions according to SEQ ID No.: 84 - 86; and/or
b. A variable heavy chain sequence comprising at least one of the CDR regions according to SEQ ID No.: 87 - 89.

134. The antibody or fragment thereof according to any of the preceding claims, wherein said CD3 antigen and said CD16 and/or a CD56 antigen are expressed on NKT cells.

135. The antibody or fragment thereof according to any of the preceding claims, wherein said antibody or fragment thereof is capable of binding to said CD3 antigen and simultaneously to said CD16 and/or CD56 antigen.
